(19) United States
(12) Patent Application Publication
(10) Pub. No.: US 2025/0361219 A1
(43) Pub. Date: Nov. 27, 2025

(54) SYSTEMS AND METHODS FOR SEPARATION OF CHEMICAL SPECIES, SUCH AS CANNABINOIDS, USING MULTIPLE LIQUID PHASES

(71) Applicant: Zaiput Flow Technologies LLC, Waltham, MA (US)

(72) Inventors: Lorenzo Milani, Woburn, MA (US); Trevor Charles Murray, Everett, MA (US); Robert Viano, Medford, MA (US); Andrea Adamo, Cambridge, MA (US); Róbert Örkényi, Cambridge (GB)

(73) Assignee: Zaiput Flow Technologies LLC, Waltham, MA (US)

(21) Appl. No.: 18/874,802

(22) PCT Filed: Jun. 14, 2023
(86) PCT No.: PCT/US2023/025273

§ 371 Date: Dec. 13, 2024

Related U.S. Application Data
(63) Continuation-in-part of application No. 17/840,884, filed on Jun. 15, 2022.
(63) Continuation-in-part of application No. 17/840,914, filed on Jun. 15, 2022, now Pat. No. 11,548,864.
(63) Continuation-in-part of application No. 18/093,910, filed on Jan. 6, 2023, now Pat. No. 12,187,695.
(63) Continuation of application No. 17/840,914, filed on Jun. 15, 2022, now Pat. No. 11,548,864.
(60) Provisional application No. 63/352,587, filed on Jun. 15, 2022.

(51) Int. Cl. C07D 311/78

(52) U.S. Cl. CPC C07D 311/78

(57) ABSTRACT

The present disclosure is related to the separation of chemical species using multiple liquid phases. The present disclosure is also related to the continuous liquid-liquid chromatographic separation of chemical species using multiple liquid phases and related systems and articles.

### RELATED APPLICATIONS

This application claims priority under 35 U.S.C. § 119 (e) to U.S. Provisional Patent Application No. 63/352,587, filed Jun. 15, 2022, and entitled “Systems and Methods for Separation of Chemical Species, such as Cannabinoids, using Multiple Liquid Phases,” which is incorporated herein by reference in its entirety for all purposes. This application is also a continuation-in-part of U.S. patent application Ser. No. 17/840,884, filed Jun. 15, 2022, and entitled “Continuous Liquid-Liquid Chromatographic Separation of Chemical Species Using Multiple Liquid Phases and Related Systems and Articles,” which is incorporated herein by reference in its entirety for all purposes. This application is also a continuation-in-part of U.S. patent application Ser. No. 18/093,910, filed Jan. 6, 2023, and entitled “Separation of Chemical Species Using Multiple Liquid Phases and Related Systems,” which is a continuation of U.S. patent application Ser. No. 17/840,914, filed Jun. 15, 2022, and entitled “Separation of Chemical Species Using Multiple Liquid Phases and Related Systems,” each of which is incorporated herein by reference in its entirety for all purposes.

### TECHNICAL FIELD

Separation of chemical species using multiple liquid phases and related systems are generally described. Continuous liquid-liquid chromatographic separation of chemical species using multiple liquid phases and related systems and articles are also generally described.

### SUMMARY

The present disclosure is related to the separation of chemical species using multiple liquid phases. Related systems and articles are also described. The present disclosure is also related to the continuous liquid-liquid chromatographic separation of chemical species using multiple liquid phases and related systems and articles. The subject matter of the present invention involves, in some cases, interrelated products, alternative solutions to a particular problem, and/or a plurality of different uses of one or more systems and/or articles.

In certain aspects, methods of separating delta-9-tetrahydrocannabinol from delta-8-tetrahydrocannabinol are provided. In some embodiments, the method comprises exposing a mixture comprising the delta-9-tetrahydrocannabinol and the delta-8-tetrahydrocannabinol to a heterogeneous liquid mixture, wherein the heterogeneous liquid mixture comprises a first liquid phase and a second liquid phase, wherein: the mole fraction of the delta-9-tetrahydrocannabinol relative to the sum of the delta-9-tetrahydrocannabinol and the delta-8-tetrahydrocannabinol in the first liquid phase is greater than the mole fraction of the delta-9-tetrahydrocannabinol relative to the sum of the delta-9-tetrahydrocannabinol and the delta-8-tetrahydrocannabinol in the mixture; and the mole fraction of the delta-8-tetrahydrocannabinol relative to the sum of the delta-8-tetrahydrocannabinol and the delta-9-tetrahydrocannabinol in the second liquid phase is greater than the mole fraction of the delta-8-tetrahydrocannabinol relative to the sum of the delta-8-tetrahydrocannabinol and the delta-9-tetrahydrocannabinol in the mixture.

In some embodiments, the method comprises exposing a mixture comprising the delta-9-tetrahydrocannabinol and the delta-8-tetrahydrocannabinol to a heterogeneous liquid mixture, wherein the heterogeneous liquid mixture comprises a first liquid phase and a second liquid phase, such that the delta-9-tetrahydrocannabinol preferentially associates with the first liquid phase and the delta-8-tetrahydrocannabinol preferentially associates with the second liquid phase.

In certain aspects, methods of separating delta-9-tetrahydrocannabinol from one or more additional cannabinoids are provided. In some embodiments, the method comprises exposing a mixture comprising the delta-9-tetrahydrocannabinol and the one or more additional cannabinoids to a heterogeneous liquid mixture, wherein the heterogeneous liquid mixture comprises a first liquid phase and a second liquid phase, wherein: the mole fraction of the delta-9-tetrahydrocannabinol relative to the sum of the delta-9-tetrahydrocannabinol and the one or more additional cannabinoids in the first liquid phase is greater than the mole fraction of the delta-9-tetrahydrocannabinol relative to the sum of the delta-9-tetrahydrocannabinol and the one or more additional cannabinoids in the mixture; and the mole fraction of the one or more additional cannabinoids relative to the sum of the one or more additional cannabinoids and the delta-9-tetrahydrocannabinol in the second liquid phase is greater than the mole fraction of the one or more additional cannabinoids relative to the sum of the one or more additional cannabinoids and the delta-9-tetrahydrocannabinol in the mixture.

In some embodiments, the method comprises exposing a mixture comprising the delta-9-tetrahydrocannabinol and the one or more additional cannabinoids to a heterogeneous liquid mixture, wherein the heterogeneous liquid mixture comprises a first liquid phase and a second liquid phase, such that the delta-9-tetrahydrocannabinol preferentially associates with the first liquid phase and the one or more additional cannabinoids preferentially associates with the second liquid phase.

Certain aspects are related to ingestible compositions. In some embodiments, the ingestible composition comprises delta-9-tetrahydrocannabinol and delta-8-tetrahydrocannabinol, wherein: the ingestible composition has a volume of at least 1 mm³; a molar ratio of delta-9-tetrahydrocannabinol to delta-8-tetrahydrocannabinol within the ingestible composition is greater than or equal to 3:1; and the amount of delta-9-tetrahydrocannabinol within the ingestible composition is at least 0.01 wt %. In some embodiments, the ingestible composition comprises delta-9-tetrahydrocannabinol and one or more additional cannabinoids, wherein: the ingestible composition has a volume of at least 1 mm³; a molar ratio of delta-9-tetrahydrocannabinol to the one or more additional cannabinoids of greater than or equal to 3:1; and delta-9-tetrahydrocannabinol within the ingestible composition is at least 0.01 wt %.

In some aspects, liquid-liquid chromatographic separator systems are provided. In some embodiments, the liquid-liquid chromatographic separator system comprises three or more separator stages, wherein the three or more separator stages are arranged in series with one another from a first separator stage to a last separator stage, with one or more intermediate separator stages positioned between the first separator stage and the last separator stage, wherein each of the three or more separator stages comprises a liquid inlet and two liquid outlets; and a feed liquid inlet configured to receive a feed liquid stream comprising a first solute and a second solute; wherein: the first separator stage comprises: a first liquid inlet configured to receive liquid comprising at least a portion of the first solute and at least a portion of the second solute, a liquid outlet configured to output a liquid having a mole fraction of the first solute relative to the sum of the first solute and the second solute that is larger than a mole fraction of the first solute relative to the sum of the first solute and the second solute in the liquid received by the first liquid inlet of the first separator stage, and a liquid outlet configured to output a liquid having a mole fraction of the second solute relative to the sum of the first solute and the second solute that is larger than a mole fraction of the second solute relative to the sum of the first solute and the second solute in the feed liquid stream; and the last separator stage comprises: a last liquid inlet configured to receive a liquid comprising at least a portion of the first solute and at least a portion of the second solute, a liquid outlet configured to output a liquid having a mole fraction of the first solute relative to the sum of the first solute and the second solute that is larger than a mole fraction of the first solute relative to the sum of the first solute and the second solute in the feed liquid stream, and a liquid outlet configured to output a liquid having a mole fraction of the second solute relative to the sum of the first solute and the second solute that is larger than a mole fraction of the second solute relative to the sum of the first solute and the second solute in the liquid received by the last liquid inlet.

In certain aspects, methods are provided. In some embodiments, the method comprises transporting a feed liquid stream comprising a first solute and a second solute into a feed liquid inlet of a liquid-liquid chromatographic separator system, wherein the liquid-liquid chromatographic separator system comprises three or more separator stages arranged in series with one another from a first separator stage to a last separator stage, with one or more intermediate stages positioned between the first separator stage and the last separator stage; transporting a liquid comprising at least a portion of the first solute and at least a portion of the second solute into a first liquid inlet of a first separator stage, such that the first separator stage produces: a liquid having a mole fraction of the first solute relative to the sum of the first solute and the second solute that is larger than a mole fraction of the first solute relative to the sum of the first solute and the second solute in the liquid received by the first liquid inlet of the first separator stage, and a liquid having a mole fraction of the second solute relative to the sum of the first solute and the second solute that is larger than a mole fraction of the second solute relative to the sum of the first solute and the second solute in the feed liquid stream; and transporting a liquid comprising at least a portion of the first solute and at least a portion of the second solute into a last liquid inlet of the last separator stage, such that the last separator stage produces: a liquid having a mole fraction of the first solute relative to the sum of the first solute and the second solute that is larger than a mole fraction of the first solute relative to the sum of the first solute and the second solute in the feed liquid stream, and a liquid having a mole fraction of the second solute relative to sum of the first solute and the second solute that is larger than a mole fraction of the second solute relative to the sum of the first solute and the second solute in the liquid received by the last liquid inlet.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1D are schematic illustrations showing a method for separating delta-9-tetrahydrocannabinol from one or more cannabinoids, according to some embodiments.

FIG. 1E is a schematic illustration showing a multi-stage separation process, according to certain embodiments.

FIG. 2A is a schematic illustration showing a liquid-liquid chromatographic separator system comprising three or more separator stages, according to some embodiments;

FIG. 2B is a schematic illustration showing a liquid-liquid chromatographic separator system comprising five or more separator stages, according to some embodiments;

FIG. 3 is a schematic illustration showing a liquid-liquid chromatographic separator system comprising six separator stages, according to some embodiments;

FIG. 4 is a schematic illustration showing a porous medium-based fluidic separator, according to some embodiments;

FIG. 5 is a schematic of a theoretical setup with N extraction stages connected in a countercurrent manner with the feed entering the system at stage F containing compounds A and B, according to some embodiments;

FIG. 6 is a photograph showing a 5-stage multi-stage extraction platform for separation of compounds, according to some embodiments;

FIG. 7 is a photograph showing a 5-stage pilot plant scale of the multi-stage extraction platform shown in FIG. 6, according to some embodiments;

FIGS. 8A-8D are graphs showing experimental separation data for a 5-stage system with varying feed stage location, ratio of feed to fresh organic, and aqueous to organic phase ratio, according to some embodiments;

FIGS. 9A-9D are graphs showing experimental separation data for a 10-stage system with varying feed stage location, ratio of feed to fresh organic, and aqueous to organic phase ratio, according to some embodiments;

FIGS. 10A-10C are graphs showing experimental extraction efficiencies and outlet purities of the separation of compounds present in depolymerized lignin; according to some embodiments; and

FIGS. 11A-11C are graphs showing experimental extraction efficiencies and outlet purities for the separation of R/S-propranolol, according to some embodiments.

### DETAILED DESCRIPTION

Separation of chemical species using multiple liquid phases and related systems are generally described. Certain aspects of the present disclosure are directed to the discovery that the use of certain heterogeneous liquid mixtures can allow for highly specific and targeted separation of a specific cannabinoid (e.g., delta-9-tetrahydrocannabinol) from one or more additional cannabinoids in a mixture. Certain embodiments are related to the discovery that the use of a heterogeneous liquid mixture comprising a first liquid phase, e.g., such as a liquid comprising an amide group, and a second liquid phase immiscible with the first liquid phase, e.g., such as a non-polar hydrocarbon, can provide, in certain instances, one or more of a variety of operational advantages. Such operational advantages include, but are not limited to, a continuous extraction process, a high extraction efficiency associated with a specific cannabinoid, reduced amount of extraction liquid and/or reduced number of extraction stages associated with the separation process. Some embodiments are related to the discovery that effective separation of a specific cannabinoid (e.g., delta-9-tetrahydrocannabinol) can be achieved by using liquids that provide different partition coefficients of the specific cannabinoid and the one or more additional cannabinoids in the heterogeneous liquid mixture. It has also been recognized, within the context of the present disclosure, that the methods described herein can be advantageously employed in the purification of cannabinoid oils obtained from raw biomass. Compared to conventional methods, the methods described herein can allow one to target a specific cannabinoid (e.g., delta-9-tetrahydrocannabinol), use less solvent(s), and/or reduce overall operational costs associated with the separation process.

In some embodiments, a method for separating delta-9-tetrahydrocannabinol from one or more additional cannabinoids is described. The method, according to some embodiments, may be employed for separating delta-9-tetrahydrocannabinol from any of a variety of cannabinoids. For example, in one set of embodiments, the method may be employed for separating delta-9-tetrahydrocannabinol from delta-8-tetrahydrocannabinol, a constitutional (e.g., structural) isomer of delta-9-tetrahydrocannabinol. Alternatively or additionally, the method may be employed for separating delta-9-tetrahydrocannabinol from another cannabinoid, such as cannabidiol (CBD). Alternatively or additionally, the method may be employed for separating delta-9-tetrahydrocannabinol from a combination of various cannabinoids (e.g., delta-8-tetrahydrocannabinol, cannabidiol, etc.). Non-limiting examples of additional cannabinoids from which delta-9-tetrahydrocannabinol may be separated are described in more detail below.

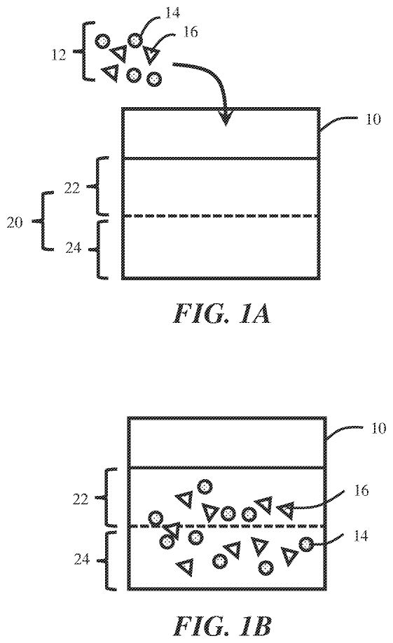

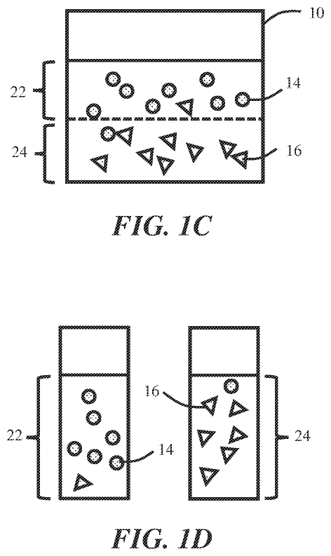

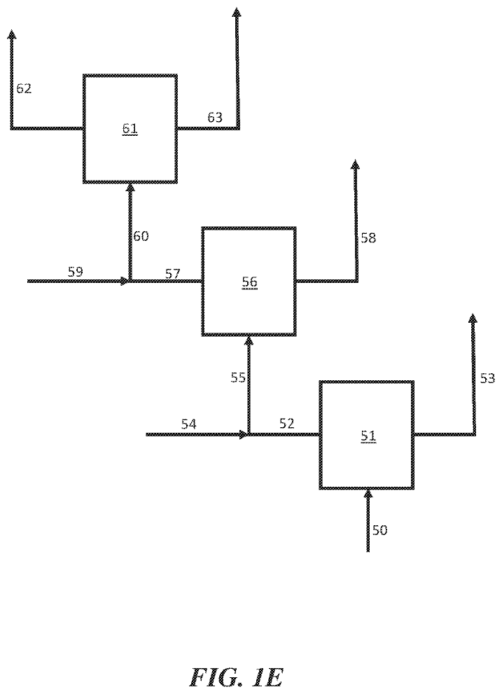

FIGS. 1A-1D are schematic illustrations of one such non-limiting method that can be used to separate delta-9-tetrahydrocannabinol from one or more additional cannabinoids, according to some embodiments. These figures are referred to throughout the disclosure below.

The method, in some embodiments, comprises exposing a mixture comprising the delta-9-tetrahydrocannabinol and the one or more additional cannabinoids to a heterogeneous liquid mixture. The delta-9-tetrahydrocannabinol may have a chemical structure as shown in formula (I):

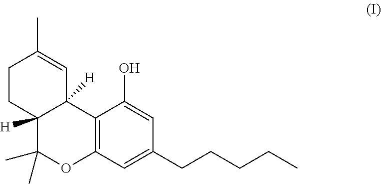 [SMILES: CCCCCc1cc(O)c2c(c1)OC(C)(C)[C@@H]1CCC(C)=C[C@@H]21]

In some embodiments, the delta-9-tetrahydrocannabinol in the mixture may comprise one or more stereoisomers (i.e., spatial isomers) of delta-9-tetrahydrocannabinol. The one or more stereoisomers of delta-9-tetrahydrocannabinol may include conformational isomers of delta-9-tetrahydrocannabinol and/or configurational isomers of delta-9-tetrahydrocannabinol. The configurational isomers of delta-9-tetrahydrocannabinol may include enantiomers and/or diastereomers of delta-9-tetrahydrocannabinol. Non-limiting examples of delta-9-tetrahydrocannabinol include (−)-delta-9-trans-tetrahydrocannabinol (e.g., as shown in formula (II)), (+)-delta-9-trans-tetrahydrocannabinol (e.g., as shown in formula (III)), (−)-delta-9-cis-tetrahydrocannabinol (e.g., as shown in formula (IV)), and (+)-delta-9-cis-tetrahydrocannabinol (e.g., as shown in formula (V)).

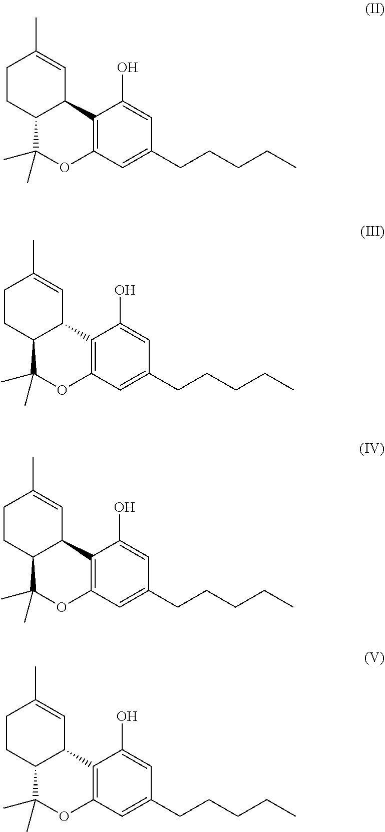

It should be noted that the delta-9-tetrahydrocannabinol described herein does not include constitutional (i.e., structural) isomers of delta-9-tetrahydrocannabinol. Non-limiting examples of constitutional (i.e., structural) isomers of delta-9-tetrahydrocannabinol include delta-8-tetrahydrocannabinol, delta-7-tetrahydrocannabinol, delta-10-tetrahydrocannabinol, delta-6a,7-tetrahydrocannabinol, delta-6a,10a-tetrahydrocannabinol, etc. It should also be noted that the delta-9-tetrahydrocannabinol described herein does not include acid forms of delta-9-tetrahydrocannabinol.

The mixture may include any of a variety of additional cannabinoids. Specific non-limiting examples of additional cannabinoids include delta-8-tetrahydrocannabinol (e.g., as shown in formula (VI)), cannabidiol (e.g., as shown in formula (VII)), other constitutional isomers of delta-9-tetrahydrocannabinol described herein, cannabigerol, cannabinol, and cannabichromene. In some embodiments, the delta-8-tetrahydrocannabinol, a constitutional isomer of delta-9-tetrahydrocannabinol, may include one or more stereoisomers of delta-8-tetrahydrocannabinol. Non-limiting examples of delta-8-tetrahydrocannabinol include (−)-delta-8-trans-tetrahydrocannabinol, (+)-delta-8-trans-tetrahydrocannabinol, (−)-delta-8-cis-tetrahydrocannabinol, and/or (+)-delta-8-cis-tetrahydrocannabinol. The method described herein may be employed for separating delta-9-tetrahydrocannabinol from one or more of the additional cannabinoids described herein.

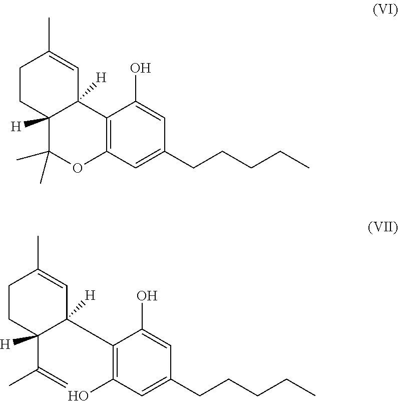 [SMILES: CCCCCc1cc(O)c2c(c1)OC(C)(C)[C@@H]1CCC(C)=C[C@@H]21 ; C=C(C)[C@@H]1CCC(C)=C[C@H]1c1c(O)cc(CCCCC)cc1O]

The phrase “heterogeneous liquid mixture” is generally used herein to refer to a liquid mixture comprising two or more distinct liquid phases. The first liquid phase and the second liquid phase may be, in some embodiments, immiscible with each other. The two or more distinct liquid phases may, in some embodiments, have a low mutual solubility with each other. For example, in some embodiments, the two or more distinct liquid phases have a mutual solubility of less than or equal to 200 mg/mL, less than or equal to 100 mg/mL, less than or equal to 50 mg/mL, less than or equal to 10 mg/mL, less than or equal to 1 mg/mL, less than or equal to 0.1 mg/mL, less than or equal to 0.001 mg/mL, less than or equal to 0.0001 mg/mL, or less than or equal to 0.00001 mg/mL (and/or, as little as 0.00001 mg/mL, as little as 0.000001 mg/mL, or less) at the temperature at which the separation is carried out. In some embodiments, the two or more distinct liquid phases have a mutual solubility of less than or equal to 200 mg/mL, less than or equal to 100 mg/mL, less than or equal to 50 mg/mL, less than or equal to 10 mg/mL, less than or equal to 1 mg/mL, less than or equal to 0.1 mg/mL, less than or equal to 0.001 mg/mL, less than or equal to 0.0001 mg/mL, or less than or equal to 0.00001 mg/mL (and/or, as little as 0.00001 mg/mL, as little as 0.000001 mg/mL, or less) at 20° C. For example, in one set of embodiments, the heterogeneous liquid mixture may comprise a first liquid phase and a second liquid phase that is immiscible with the first liquid phase, e.g., such as polar liquid and a non-polar liquid. In some embodiments, the first liquid phase and the second liquid phase have mutual solubilities falling within any of the ranges outlined above.

FIG. 1A illustrates an example of exposing a mixture comprising delta-9-tetrahydrocannabinol and one or more additional cannabinoids (e.g., delta-8-tetrahydrocannabinol, cannabidiol, etc.) to a heterogeneous liquid mixture. As shown in FIG. 1A, mixture 12 comprising delta-9-tetrahydrocannabinol 14 and one or more additional cannabinoids 16 is exposed to heterogeneous liquid mixture 20. Heterogeneous liquid mixture 20 may comprise two or more immiscible liquid phases, e.g., such as first liquid phase 22 and second liquid phase 24.

The first liquid phase and the second liquid phase may be present in the heterogeneous liquid mixture in any of a variety of mass ratios. For example, in some embodiments, a mass ratio of the first liquid phase to the second liquid phase in the mixture may be greater than or equal to 5:95, greater than or equal to 10:90, greater than or equal to 20:80, greater than or equal to 30:70, greater than or equal to 40:60, greater than or equal to 50:50, greater than or equal to 60:40, greater than or equal to 70:30, greater than or equal to 80:20, or greater than or equal to 90:10. In some embodiments, a mass ratio of the first liquid phase to the second liquid phase in the mixture may be less than or equal to 95:5, less than or equal to 90:10, less than or equal to 80:20, less than or equal to 70:30, less than or equal to 60:40, less than or equal to 50:50, less than or equal to 40:60, less than or equal to 30:70, less than or equal to 20:80, or less than or equal to 10:90. Combinations of the above-referenced ranges are also possible (e.g., greater than or equal to 5:95 and less than or equal to 95:5). Other ranges are also possible. As would be understood by one of ordinary skill in the art, when a mass ratio of A:B is “greater than or equal to 10:90,” it means that, when the mass of component A that is present is divided by the mass of component B that is present, the resulting value is greater than or equal to 10/90 (i.e., greater than or equal to 0.111 repeating). Similarly, when a mass ratio of A:B is “less than or equal to 90:10,” it that means that, when the mass of component A that is present is divided by the mass of component B that is present, the resulting value is less than or equal to 90/10 (i.e., less than or equal to 9).

The first liquid phase can be, in some embodiments, water soluble. In some embodiments, the first liquid phase may comprise at least one (e.g., at least one, at least two, at least three, etc.) liquid(s) comprising an amide group. As used herein, a liquid comprising an amide group is also generally referred to as “an amide-containing liquid.” The first liquid phase, in some embodiments, may be miscible with water. In some embodiments, the first liquid phase comprises a polar aprotic solvent comprising an amide group.

The first liquid phase may comprise any of variety of appropriate amide-containing liquids. In some embodiments, the first liquid phase includes at least one liquid having a chemical structure shown in formula (VIII):

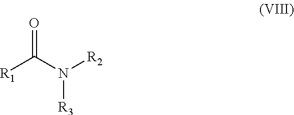

where R₁ is selected from hydrogen and C₁₋₄ aliphatic having a total of 1 to 4 carbon atoms, and where R₂ and R₃ can be the same or different and each is independently selected from hydrogen and C₁₋₄ aliphatic having a total of 1 to 4 carbon atoms. In some embodiments, the first liquid phase comprises at least one liquid having a chemical structure shown in formula (VIII) where R₁ is selected from hydrogen, C₁₋₄ alkyl having a total of 1 to 4 carbon atoms, C₁₋₄ alkenyl having a total of 1 to 4 carbon atoms, and C₁₋₄ alkyl having a total of 1 to 4 carbon atoms, and where R₂ and R₃ can be the same or different and each is independently selected from hydrogen, C₁₋₄ alkyl having a total of 1 to 4 carbon atoms, C₁₋₄ alkenyl having a total of 1 to 4 carbon atoms, and C₁₋₄ alkyl having a total of 1 to 4 carbon atoms. In some embodiments, the first liquid phase comprises at least one liquid having a chemical structure shown in formula (VIII) where R₁ is selected from hydrogen and C₁₋₄ alkyl having a total of 1 to 4 carbon atoms, and where R₂ and R₃ can be the same or different and each is independently selected from hydrogen and C₁₋₄ alkyl having a total of 1 to 4 carbon atoms. In some embodiments, the first liquid phase comprises at least one liquid having a chemical structure shown in formula (VIII) where R₁ is selected from hydrogen and C₁₋₄ alkyl having a total of 1 to 4 carbon atoms, and where R₂ and R₃ can be the same or different and each is independently selected from hydrogen and C₁₋₃ alkyl having a total of 1 to 3 carbon atoms.

Specific non-limiting examples of amide-containing liquids include formamide, acetamide, propanamide, butanamide, dimethyl formamide, diethyl formamide, dibutyl formamide, methyl formamide, dimethyl acetamide, diethyl acetamide, dimethyl propanamide, diethyl propanamide, dimethyl butanamide, and/or diethyl butanamide.

In some embodiments, the first liquid phase is a mixture comprising at least two (e.g., at least three, at least four, etc.) amide-containing liquids. For example, the first liquid phase may comprise an amide-containing liquid of a first type and an amide-containing liquid of a second type. In some embodiments, the amide-containing liquid of a first type has a lower polarity compared to the amide-containing liquid of a second type. An amide-containing liquid of a first type may have a structure as shown in formula (VIII), where R₁ is selected from hydrogen and C₁₋₄ aliphatic having 1 to 4 total carbon atoms, and where each of R₂ and R₃ is a hydrogen. In some embodiments, an amide-containing liquid of a first type may have a structure as shown in formula (VIII), where R₁ is selected from hydrogen and C₁₋₄ alkyl having 1 to 4 total carbon atoms, and where each of R₂ and R₃ is a hydrogen. In some embodiments, the first liquid phase may comprise two or more amide-containing liquids of a first type.

An amide-containing liquid of a second type may have a structure as shown in formula (VIII), where R₁ is selected from hydrogen and C₁₋₄ aliphatic having 1 to 4 total carbon atoms, and where R₂ and R₃ can be the same or different and each is independently a C₁₋₄ aliphatic having 1 to 4 total carbon atoms. For example, in some embodiments, an amide-containing liquid of a second type may have a structure as shown in formula (VIII), where R₁ is selected from hydrogen and C₁₋₄ alkyl having 1 to 4 total carbon atoms, and where R₂ and R₃ can be the same or different and each is independently a C₁₋₄ alkyl having 1 to 4 total carbon atoms. Alternatively or additionally, in some embodiments, an amide-containing liquid of a second type may have a structure as shown in formula (VIII), where R₁ is selected from hydrogen and C₁₋₄ aliphatic having 1 to 4 total carbon atoms, and where R₂ and R₃ are different and each is independently selected from a hydrogen and a C₁₋₄ aliphatic having 1 to 4 total carbon atoms. For example, an amide-containing liquid of a second type may have a structure as shown in formula (VIII), where R₁ is selected from hydrogen and C₁₋₄ alkyl having 1 to 4 total carbon atoms, and where R₂ and R₃ are different and each is independently selected from a hydrogen and a C₁₋₄ alkyl having 1 to 4 total carbon atoms. In some embodiments, the first liquid phase may comprise two or more amide-containing liquids of a second type.

Non-limiting examples of the amide-containing liquid of a first type include formamide, acetamide, propanamide, and/or butanamide. Non-limiting examples of the amide-containing liquid of a second type include dimethyl formamide, diethyl formamide, dibutyl formamide, methyl formamide, dimethyl acetamide, diethyl acetamide, dimethyl propanamide, diethyl propanamide, dimethyl butanamide, and/or diethyl butanamide.

In embodiments in which the first liquid phase is a mixture comprising an amide-containing liquid of a first type (e.g., formamide) and an amide-containing liquid of a second type (e.g., dimethylformamide), the two types of amide-containing liquid may be present in any of a variety of appropriate mass ratios. For example, in some embodiments, a mass ratio of the amide-containing liquid of a first type (e.g., formamide) to the amide-containing liquid of a second type (e.g., dimethylformamide) in the mixture may be greater than or equal to 20:80, greater than or equal to 30:70, greater than or equal to 33.3:66.6, greater than or equal to 40:60, greater than or equal to 45:55, greater than or equal to 50:50, greater than or equal to 55:45, greater than or equal to 60:40, greater than or equal to 66.6:33.3, or greater than or equal to 70:30. In some embodiments, a mass ratio of the amide-containing liquid of a first type (e.g., formamide) to the amide-containing liquid of a second type (e.g., dimethylformamide) in the mixture may be less than or equal to 80:20, less than or equal to 70:30, less than or equal to 66.6:33.3, less than or equal to 60:40, less than or equal to 55:45, less than or equal to 50:50, less than or equal to 45:55, less than or equal to 40:60, less than or equal to 33.3:66.6, or less than or equal to 30:70. Combinations of the above-referenced ranges are also possible (e.g., greater than or equal to 20:80 and less than or equal to 80:20, or greater than or equal to 40:60 and less than or equal to 60:40). Other ranges are also possible.

In some embodiments, the first liquid may comprise more than one amide-containing liquid of a first type and more than one amide-containing liquids of a second type. For example, as a non-limiting embodiment, the first liquid may comprise an amide-containing liquid of a first type (e.g., formamide) and a mixture of two amide-containing liquids of a second type (e.g., dimethyl formamide and dibutyl formamide).

In accordance with certain embodiments, the heterogeneous liquid mixture comprises a second liquid phase. The second liquid phase can be, in accordance with some embodiments, a water insoluble organic phase. The second liquid phase, in certain embodiments, comprises at least one (e.g., at least two, at least three, etc.) aliphatic hydrocarbon(s). As used herein, the term “aliphatic” refers to alkyl, alkenyl, alkynyl, and carbocyclic groups. The term “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, the second liquid phase comprises a C₃₋₂₀ aliphatic hydrocarbon having a total of 3 to 20 carbon atoms. For example, “C₃₋₂₀ aliphatic” may encompass, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₃₋₂₀, C₃₋₁₈, C₃₋₁₆, C₃₋₁₄, C₃₋₁₂, C₃₋₁₀, C₃₋₈, C₃₋₆, C₃₋₄, C₄₋₂₀, C₄₋₁₈, C₄₋₁₆, C₄₋₁₄, C₄₋₁₂, C₄₋₁₀, C₄₋₈, C₄₋₆, C₄₋₅, C₆₋₂₀, C₆₋₁₈, C₆₋₁₆, C₆₋₁₄, C₆₋₁₂, C₆₋₁₀, C₆₋₈, C₆₋₇, C₈₋₂₀, C₈₋₁₈, C₈₋₁₆, C₈₋₁₄, C₈₋₁₂, C₈₋₁₀, C₈₋₉, C₁₀₋₂₀, C₁₀₋₁₈, C₁₀₋₁₆, C₁₀₋₁₄, C₁₀₋₁₂, C₁₀₋₁₁, C₁₂₋₂₀, C₁₂₋₁₈, C₁₂₋₁₆, C₁₂₋₁₄, C₁₂₋₁₃, C₁₄₋₂₀, C₁₄₋₁₈, C₁₄₋₁₆, C₁₄₋₁₅, C₁₆₋₂₀, C₁₆₋₁₈, C₁₆₋₁₇, C₁₈₋₂₀, C₁₈₋₁₉, and C₁₉₋₂₀ aliphatic. In some embodiments, the C₃₋₂₀ aliphatic hydrocarbon is branched or unbranched, saturated or unsaturated, acyclic or cyclic. In some embodiments, the C₃₋₂₀ aliphatic hydrocarbon comprises an alkyl, an alkenyl, an alkynyl, a cycloalkyl, a cycloalkenyl, and/or a cycloalkynyl group having a total of 3 to 20 carbon atoms. In some embodiments, the aliphatic hydrocarbon is unsubstituted and/or does not include a heteroatom. For example, in some embodiments, a liquid of the second liquid phase does not include a substituted aliphatic hydrocarbon and/or a heteroaliphatic hydrocarbon, e.g., an aliphatic hydrocarbon comprising at least one heteroatom. In some embodiments, a liquid of the second liquid phase comprises a C₃₋₂₀ aliphatic hydrocarbon that is immiscible with the first liquid phase.

The second liquid phase may include any of a variety of suitable C₃₋₂₀ aliphatic hydrocarbons. Non-limiting examples of C₃₋₂₀ aliphatic hydrocarbons that can be used in the second liquid phase include alkanes, alkenes, alkynes, cycloalkanes, cycloalkene, cycloalkene, and/or cycloalkynes. Non-limiting examples of liquids that can be used in the second liquid phase include pentane, pentene, pentyne, cyclopentane, cyclopentene, cyclopentyne, hexane, hexene, hexyne, cyclohexane, cyclohexene, cyclohexyne, heptane, heptene, heptyne, cycloheptane, cycloheptene, cycloheptyne, dodecane, dodecene, dodecyne, cyclododecane, cyclododecene, and/or cyclododecyne.

Certain embodiments of the present disclosure comprise separating delta-9-tetrahydrocannabinol from one or more additional cannabinoids (e.g., delta-8-tetrahydrocannabinol, cannabidiol, etc.) using a heterogeneous liquid mixture, such as any of the mixtures described above or elsewhere herein.

In association with certain of the embodiments described herein, certain liquids are said to be “enriched” in a first solute or a second solute, relative to another liquid. In this context, a first liquid is said to be “enriched” in the first solute relative to a second liquid if the mole fraction of the first solute relative to the sum of the first solute and the second solute in the first liquid is higher than the mole fraction of the first solute relative to the sum of the first solute and the second solute in the second liquid. Similarly, a first liquid is said to be “enriched” in the second solute relative to a second liquid if a mole fraction of the second solute relative to the sum of the first solute and the second solute in the first liquid is higher than a mole fraction of the second solute relative to the sum of the first solute and the second solute in the second liquid. In some instances in which a first liquid is enriched in a solute relative to a second liquid, it is particularly advantageous if the concentration of the solute in the first liquid is higher than the concentration of that solute in the second liquid. For example, in some embodiments, it is particularly advantageous if a separation process produces (1) a first liquid that has a higher concentration of first solute (e.g., delta-9-tetrahydrocannabinol) than the concentration of the first solute in the initial mixture and (2) a second liquid that has a higher concentration of second solute (e.g., delta-8-tetrahydrocannabinol and/or other cannabinoids that are not delta-9-tetrahydrocannabinol) than the concentration of the second solute in the initial mixture.

In certain embodiments, the method for separating delta-9-tetrahydrocannabinol from delta-8-tetrahydrocannabinol comprises exposing a the mixture comprising the delta-9-tetrahydrocannabinol and the delta-8-tetrahydrocannabinol to a heterogeneous liquid mixture such that the mole fraction of the delta-9-tetrahydrocannabinol relative to the sum of the delta-9-tetrahydrocannabinol and the delta-8-tetrahydrocannabinol in the first liquid phase is greater than the mole fraction of the delta-9-tetrahydrocannabinol relative to the sum of the delta-9-tetrahydrocannabinol and the delta-8-tetrahydrocannabinol in the mixture. In some embodiments, the mole fraction of the delta-9-tetrahydrocannabinol relative to the sum of the delta-9-tetrahydrocannabinol and the delta-8-tetrahydrocannabinol in the first liquid phase is at least 10% (or at least 25%, at least 50%, at least 100%, at least 1000%, or more) greater than the mole fraction of the delta-9-tetrahydrocannabinol relative to the sum of the delta-9-tetrahydrocannabinol and the delta-8-tetrahydrocannabinol in the mixture.

In addition, in some such embodiments, the mole fraction of the delta-8-tetrahydrocannabinol relative to the sum of the delta-8-tetrahydrocannabinol and the delta-9-tetrahydrocannabinol in the second liquid phase is greater than the mole fraction of the delta-8-tetrahydrocannabinol relative to the sum of the delta-8-tetrahydrocannabinol and the delta-9-tetrahydrocannabinol in the mixture. In some embodiments, the mole fraction of the delta-8-tetrahydrocannabinol relative to the sum of the delta-8-tetrahydrocannabinol and the delta-9-tetrahydrocannabinol in the second liquid phase is at least 10% (or at least 25%, at least 50%, at least 100%, at least 1000%, or more) greater than the mole fraction of the delta-8-tetrahydrocannabinol relative to the sum of the delta-8-tetrahydrocannabinol and the delta-9-tetrahydrocannabinol in the mixture.

To calculate a mole fraction of a first solute relative to the sum of the first solute and the second solute in a particular liquid, one would divide the number of moles of the first solute present in the liquid by the sum of the number of moles of the first solute present in the liquid and the number of moles of the second solute present in the liquid. This is shown mathematically as follows:

$${x}_{1} = \frac{{n}_{1}}{{n}_{1} + {n}_{2}}$$

where x₁ is the mole fraction of the first solute relative to the sum of the first solute and the second solute in the liquid, n₁ is the number of moles of the first solute in the liquid, and n₂ is the number of moles of the second solute in the liquid. Similarly, to calculate a mole fraction of a second solute relative to the sum of the first solute and the second solute in a particular liquid, one would divide the number of moles of the second solute present in the liquid by the sum of the number of moles of the first solute present in the liquid and the number of moles of the second solute present in the liquid. This is shown mathematically as follows:

$${x}_{2} = \frac{{n}_{2}}{{n}_{1} + {n}_{2}}$$

where x₂ is the mole fraction of the second solute relative to the sum of the first solute and the second solute in the liquid, n₁ is the number of moles of the first solute in the liquid, and n₂ is the number of moles of the second solute in the liquid.

In some embodiments, the separating may occur as a result of preferential association of the delta-9-tetrahydrocannabinol and the one or more additional cannabinoids with different liquid phases of the heterogeneous liquid mixture. For example, in some embodiments, upon exposing the mixture to the heterogeneous liquid mixture, the delta-9-tetrahydrocannabinol preferentially associates with the first liquid phase, while the one or more additional cannabinoids preferentially associate with the second liquid phase. In some such embodiments, after the preferential associations, the first liquid phase becomes enriched in the delta-9-tetrahydrocannabinol, while the second liquid phase becomes enriched in the one or more additional cannabinoids.

FIGS. 1B-1C illustrate an example of the preferential association of the delta-9-tetrahydrocannabinol and the one or more additional cannabinoids (e.g., delta-8-tetrahydrocannabinol, cannabidiol, etc.) into separate liquid phases. As shown in FIG. 1B, the mixture comprising delta-9-tetrahydrocannabinol 14 and one or more additional cannabinoids 16 has been disposed in heterogeneous liquid mixture 20 comprising first liquid phase 22 and second liquid phase 24. As shown in FIG. 1C, delta-9-tetrahydrocannabinol 14 has preferentially associated with first liquid phase 22, while one or more additional cannabinoids 16 have preferentially associated with second liquid phase 24. After the preferential association, first liquid phase 22 is enriched in delta-9-tetrahydrocannabinol 14 and second liquid phase 24 is enriched in the one or more additional cannabinoids 16.

In some embodiments, an increase in a molar ratio and/or a preferential association of the chemical species (e.g., the delta-9-tetrahydrocannabinol and the one or more additional cannabinoids) with their respective liquid phases (e.g., the first liquid phase, the second liquid phase) in the heterogeneous liquid mixture is related to the ability of the chemical species to selectively partition into the different liquid phases. For example, in a biphasic heterogeneous liquid mixture comprising a first liquid phase and a second liquid phase, a partition coefficient Kₙ may be defined for each chemical species as a measure of its ability to partition between the first liquid phase and the second liquid phase at equilibrium. For chemical species i, the partition coefficient Kᵢ may be expressed as: Kᵢ=C_{i(1st liquid phase)}/C_{i(2nd liquid phase)}, which is a ratio of the concentration of chemical species i in the first liquid phase (C_{i, 1st liquid phase}) to the concentration of chemical species i in the second liquid phase (C_{i, 2st liquid phase}). In the context of the present disclosure, chemical species i may refer to delta-9-tetrahydrocannabinol or any of the one or more additional cannabinoids. The concentration of chemical species is generally expressed in terms of molarity (i.e., M, or moles per liter).

For example, in embodiments in which the mixture comprises the delta-9-tetrahydrocannabinol and the one or more additional cannabinoids, the delta-9-tetrahydrocannabinol may have a partition coefficient K_{d9,THC}, which, as described above, is expressed as a ratio of the concentration of the delta-9-tetrahydrocannabinol in the first liquid phase to the concentration of the delta-9-tetrahydrocannabinol in the second liquid phase at equilibrium (e.g., K_{d9,THC=}C_{d9, THC (1st liquid phase)}/C_{d9, THC (2nd liquid phase)}). Similarly, each of the one or more additional cannabinoids may individually have and/or collectively have a partition coefficient K_{cnbd, i}, where K_{cnbd, i} is expressed as a ratio of the concentration of the one or more additional cannabinoids in the first liquid phase to the concentration of the one or more additional cannabinoids in the second liquid phase (e.g., K_{cnbd, i}=C_{cnbd, i (1st liquid phase)}/C_{cnbd, i (2nd liquid phase)}). For example, in embodiments in which the one or more additional cannabinoids comprise delta-8-tetrahydrocannabinol, a partition coefficient K_{d8,THC} for the delta-8-tetrahydrocannabinol between the first liquid phase and the second liquid phase may be expressed a ratio of the concentration of the delta-8-tetrahydrocannabinol in the first liquid phase to the concentration of the delta-8-tetrahydrocannabinol in the second liquid phase (e.g., C_{d8, THC (1st liquid phase)}/C_{d8, THC (2nd liquid phase)}). As another example, in embodiments in which the one or more additional cannabinoids comprise cannabidiol, a partition coefficient K_{CBD} for cannabidiol between the first liquid phase and the second liquid phase may be expressed as a ratio of the concentration of the cannabidiol in the first liquid phase to the concentration of the cannabidiol in the second liquid phase (e.g., C_{CBD (1st liquid phase)}/C_{CBD (2nd liquid phase)}). The ranges described herein for the partition coefficient of K_{cnbd, i} between the first liquid phase and the second liquid phase may, in certain embodiments, be the partition coefficient of delta-8-tetrahydrocannabinol (K_{d8, THC}) between the first liquid phase and the second liquid phase, the partition coefficient of cannabidiol (K_{CBD}) between the first liquid phase and the second liquid phase may, and/or the partition coefficient of any other cannabinoid that is not delta-8-tetrahydrocannabinol between the first liquid phase and the second liquid phase.

The delta-9-tetrahydrocannabinol may have any of a variety of appropriate partition coefficients K_{d9,THC} between the first liquid phase and the second liquid phase. In some embodiments, the delta-9-tetrahydrocannabinol may have a partition coefficient K_{d9,THC} between the first liquid phase and the second liquid phase of greater than or equal to 0.1, greater than or equal to 0.2, greater than or equal to 0.3, greater than or equal to 0.5, greater than or equal to 0.75, greater than or equal to 1, greater than or equal to 1.05, greater than or equal to 1.1, greater than or equal to 1.15, greater than or equal to 1.2, greater than or equal to 1.25, greater than or equal to 1.3, greater than or equal to 1.4, greater than or equal to 1.6, greater than or equal to 1.8, or greater than or equal to 2. In some embodiments, the delta-9-tetrahydrocannabinol may have a partition coefficient K_{d9,THC} between the first liquid phase and the second liquid phase of up to 2.5, up to 3, up to 4, up to 5, up to 6, up to 8, up to 10, or greater. Combinations of the above-referenced ranges are possible (e.g., greater than or equal to 0.1 and up to 10).

The one or more cannabinoids other than delta-9-tetrahydrocannabinol (e.g., delta-8-tetrahydrocannabinol, cannabidiol, etc.) may have any of a variety of appropriate partition coefficients K_{cnbd,i} (e.g., K_{d8,THC}, K_{CBD}, etc.) between the first liquid phase and the second liquid phase. In some embodiments, the one or more cannabinoids other than delta-9-tetrahydrocannabinol may have a partition coefficient K_{cnbd,i} between the first liquid phase and the second liquid phase of less than or equal to 200, less than or equal to 150, less than or equal to 100, less than or equal to 50, less than or equal to 25, less than or equal to 10, less than or equal to 8, less than or equal to 6, less than or equal to 4, less than or equal to 3, less than or equal to 2, less than or equal to 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, or less than or equal to 0.2 (and/or down to 0.01, down to 0.001, or less). For example, in some embodiments, K_{d8,THC} between the first liquid phase and the second liquid phase is less than or equal to 2, less than or equal to 1.8, less than or equal to 1.6, less than or equal to 1.5, less than or equal to 1.4, less than or equal to 1.2, less than or equal to 1.1, less than or equal to 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, or less than or equal to 0.2 (and/or down to 0.1 or less). As another example, in some embodiments, K_{CBD} between the first liquid phase and the second liquid phase is less than or equal to 200, less than or equal to 150, less than or equal to 100, less than or equal to 50, less than or equal to 25, less than or equal to 10, less than or equal to 8, less than or equal to 6, less than or equal to 4, less than or equal to 3, less than or equal to 2, less than or equal to 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, or less than or equal to 0.2 (and/or down to 0.01, down to 0.001, or less). In certain embodiments, for each cannabinoid in the mixture that is not delta-9-tetrahydrocannabinol and is not delta-8-tetrahydrocannabinol, the partition coefficient K between the first liquid phase and the second liquid phase for that cannabinoid is less than or equal to 200, less than or equal to 150, less than or equal to 100, less than or equal to 50, less than or equal to 25, less than or equal to 10, less than or equal to 8, less than or equal to 6, less than or equal to 4, less than or equal to 3, less than or equal to 2, less than or equal to 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, or less than or equal to 0.2 (and/or down to 0.01, down to 0.001, or less).

In some embodiments, it may be advantageous to select a heterogeneous liquid mixture having a relatively high ratio of the partition coefficient K_{d9,THC} of the delta-9-tetrahydrocannabinol to the partition coefficient K_{cnbd, i} (e.g., K_{d8,THC}, K_{CBD}, etc.) of the one or more additional cannabinoids (e.g., delta-8-tetrahydrocannabinol, cannabidiol, etc.). A heterogeneous liquid mixture having a relatively high ratio of K_{d9,THC} to K_{cnbd, i}. may lead to a more efficient association of the delta-9-tetrahydrocannabinol with the first liquid phase, and similarly, a more efficient association of the one or more additional cannabinoids with the second liquid phase. For example, in a heterogeneous liquid mixture having a relatively high K_{d9,THC} to K_{cnbd, i} ratio (e.g., as described below), the delta-9-tetrahydrocannabinol may exhibit a higher selectivity towards the first liquid phase compared to the one of more additional cannabinoids, and thereby result in a more efficient partitioning of the delta-9-tetrahydrocannabinol into the first liquid phase.

In some embodiments, the ratio of the partition coefficient K_{d9,THC} of the delta-9-tetrahydrocannabinol to the partition coefficient K_{cnbd, i}. (e.g., K_{d8,THC}, K_{CBD}) of the one or more additional cannabinoids (e.g., delta-8-tetrahydrocannabinol, cannabidiol, etc.) is greater than 1, greater than or equal to 1.05, greater than or equal to 1.1, greater than or equal to 1.15, greater than or equal to 1.2, greater than or equal to 1.25, greater than or equal to 1.3, greater than or equal to 1.35, greater than or equal to 1.4, greater than or equal to 1.5, greater than or equal to 2, greater than or equal to 3, greater than or equal to 5, greater than or equal to 10, greater than or equal to 50, or greater than or equal to 100 (and/or, in some embodiments, less than or equal to 100, less than or equal to 50, less than or equal to 10, less than or equal to 5, less than or equal to 3, less than or equal to 2, less than or equal to 1.5, less than or equal to 1.4, less than or equal to 1.35, less than or equal to 1.3, less than or equal to 1.25, or less). Combinations of the above-referenced ranges are possible (e.g., greater than 1 and less than or equal to 100, greater than or equal to 1.25 and less than or equal to 100, or greater than or equal to 1.3 and less than or equal to 100).

For example, in some embodiments, the ratio of the partition coefficient K_{d9,THC} of the delta-9-tetrahydrocannabinol to the partition coefficient K_{d8,THC} of the delta-8-tetrahydrocannabinol is greater than 1, greater than or equal to 1.05, greater than or equal to 1.1, greater than or equal to 1.15, greater than or equal to 1.2, greater than or equal to 1.25, greater than or equal to 1.3, greater than or equal to 1.35, greater than or equal to 1.4, greater than or equal to 1.5, greater than or equal to 2, greater than or equal to 3, greater than or equal to 5, or greater than or equal to 10 (and/or, in some embodiments, less than or equal to 10, less than or equal to 5, less than or equal to 3, less than or equal to 2, less than or equal to 1.5, less than or equal to 1.4, less than or equal to 1.35, less than or equal to 1.3, less than or equal to 1.25, or less). Combinations of the above-referenced ranges are possible (e.g., greater than 1 and less than or equal to 10, greater than or equal to 1.25 and less than or equal to 10, or greater than or equal to 1.3 and less than or equal to 10).

As another example, in some embodiments, the ratio of the partition coefficient K_{d9,THC} of the delta-9-tetrahydrocannabinol to the partition coefficient K_{CBD} of cannabidiol is greater than 1, greater than or equal to 1.05, greater than or equal to 1.1, greater than or equal to 1.15, greater than or equal to 1.2, greater than or equal to 1.25, greater than or equal to 1.3, greater than or equal to 1.35, greater than or equal to 1.4, greater than or equal to 1.5, greater than or equal to 2, greater than or equal to 3, greater than or equal to 5, greater than or equal 10, greater than or equal to 50, or greater than or equal to 100 (and/or, in some embodiments, less than or equal to 100, less than or equal to 50, less than or equal to 10, less than or equal to 5, less than or equal to 3, less than or equal to 2, less than or equal to 1.5, less than or equal to 1.4, less than or equal to 1.35, less than or equal to 1.3, less than or equal to 1.25, or less). Combinations of the above-referenced ranges are possible (e.g., greater than 1 and less than or equal to 100, greater than or equal to 1.25 and less than or equal to 100, or greater than or equal to 1.3 and less than or equal to 100).

In some embodiments, for each cannabinoid in the mixture that is not the delta-9-tetrahydrocannabinol and is not delta-8-tetrahydrocannabinol, the ratio of the partition coefficient K_{d9,THC} of the delta-9-tetrahydrocannabinol to the partition coefficient K of that cannabinoid that is not delta-9-tetrahydrocannabinol and is not delta-8-tetrahydrocannabinol is greater than 1, greater than or equal to 1.05, greater than or equal to 1.1, greater than or equal to 1.15, greater than or equal to 1.2, greater than or equal to 1.25, greater than or equal to 1.3, greater than or equal to 1.35, greater than or equal to 1.4, greater than or equal to 1.5, greater than or equal to 2, greater than or equal to 3, greater than or equal to 5, greater than or equal to 10, greater than or equal to 50, or greater than or equal to 100 (and/or, in some embodiments, less than or equal to 100, less than or equal to 50, less than or equal to 10, less than or equal to 5, less than or equal to 3, less than or equal to 2, less than or equal to 1.5, less than or equal to 1.4, less than or equal to 1.35, less than or equal to 1.3, less than or equal to 1.25, or less). Combinations of the above-referenced ranges are also possible (e.g., greater than 0.01 and less than or equal to 100, greater than or equal to 1.25 and less than or equal to 100, or greater than or equal to 1.3 and less than or equal to 100). Other ranges are also possible.

In some embodiments, it may be particularly advantageous to select a heterogeneous liquid mixture having a delta-9-tetrahydrocannabinol partition coefficient K_{d9,THC} of greater than 1 and a partition coefficient K_{cnbd,i} (e.g., K_{d8,THC}, K_{CBD}, etc.) of less than 1 for the one or more additional cannabinoids (e.g., delta-8-tetrahydrocannabinol, cannabidiol, etc.) or vice versa. In some such embodiments, the delta-9-tetrahydrocannabinol may preferentially partition to the first liquid phase over the second liquid phase, and the one or more additional cannabinoids may preferentially partition to the second liquid phase over the first liquid phase. Such a combination of partition coefficients may result in a higher extraction efficiency of the delta-9-tetrahydrocannabinol and may be associated with certain operational advantages (e.g., need for less solvent, lower number of extraction stages, etc.). For example, in some embodiments, the delta-9-tetrahydrocannabinol may have a partition coefficient K_{d9,THC} between the first liquid phase and the second liquid phase of greater than 1, greater than or equal to 1.05, greater than or equal to 1.1, greater than or equal to 1.15, greater than or equal to 1.2, greater than or equal to 1.25, greater than or equal to 1.3, greater than or equal to 1.4, greater than or equal to 1.6, greater than or equal to 1.8, greater than or equal to 2, or greater (and/or, in some embodiments, up to 2.5, up to 3, up to 4, up to 5, up to 6, up to 8, or up to 10, or more). Combinations of the above-referenced ranges are possible (e.g., greater than 1 and up to 10). Other ranges are also possible. Additionally, in some embodiments, the one or more additional cannabinoids (e.g., delta-8-tetrahydrocannabinol, cannabidiol, etc.) may have a partition coefficient K_{cnbd,i} (e.g., K_{d8,THC}, K_{CBD}) between the first liquid phase and the second liquid phase of less than 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, less than or equal to 0.2, less than or equal to 0.1, or less (and/or down to 0.01, down to 0.001, or less). Combinations of the above-referenced ranges are possible. Other ranges are also possible.

For example, in some embodiments, the delta-8-tetrahydrocannabinol may have a partition coefficient (K_{d8,THC}) between the first liquid phase and the second liquid phase of less than 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, less than or equal to 0.2, less than or equal to 0.1, or less (and/or down to 0.01, down to 0.001, or less). As another example, in some embodiments, the cannabidiol may have a partition coefficient (K_{CBD}) between the first liquid phase and the second liquid phase of less than 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, less than or equal to 0.2, less than or equal to 0.1, or less (and/or down to 0.01, down to 0.001, or less). In some embodiments, for each cannabinoid in the mixture that is not the delta-9-tetrahydrocannabinol, the partition coefficient K of that cannabinoid that is not delta-9-tetrahydrocannabinol is less than 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, less than or equal to 0.2, less than or equal to 0.1, or less (and/or down to 0.01, down to 0.001, or less). Combinations of the above-referenced ranges are possible. Other ranges are also possible.

In some embodiments, it may be advantageous to select a heterogeneous liquid mixture having a particular combination of partition coefficients (e.g., K_{d9,THC}, K_{cnbd,i}) and/or a particular ratio of between the partition coefficients (e.g., K_{d9,THC}/K_{cnbd,i}) in one or more of the ranges referenced above. For example, in some embodiments, it may be desirable to select a heterogeneous liquid mixture having a relatively high K_{d9,THC}/K_{cnbd,i} ratio (e.g., greater than 1.1, greater than or equal to 1.25, or greater than or equal to 1.3) and/or a heterogeneous liquid mixture having a particular combination of partition coefficients (e.g., a K_{d9,THC} that is greater than 1 and a K_{cnbd,i} that is less than 1 or vice versa).

The above-referenced partition coefficients (e.g., K_{d9,THC}, K_{cnbd,i}) of various cannabinoids and/or ratios thereof (e.g., K_{d9,THC}/K_{cnbd,i}) may be controlled by adjusting the types and/or relative amount of various liquids within the heterogeneous liquid mixture. For example, in embodiments in which the first liquid phase comprises a first amide-containing liquid (e.g., formamide) and a second amide-containing liquid (e.g., dimethyl formamide or methyl formamide), the relative amount (mass ratio of the of the two types of amide-containing liquid may be adjusted to vary the polarity of the first liquid phase, thereby establishing desirable partition coefficients of the various cannabinoids and/or the ratio thereof. Similarly, the type and amount of the first liquid phase and the second liquid phase may be adjusted to control the differential partitioning of various cannabinoids into different liquid phases.

In some embodiments, the method comprises separating the delta-9-tetrahydrocannabinol that associated with the first liquid phase from the one or more additional cannabinoids (e.g., delta-8-tetrahydrocannabinol, cannabidiol, etc.) that associated with the second liquid phase. FIG. 1D illustrates an example of one such set of embodiments. As shown in FIG. 1D, first liquid phase 22 containing the preferentially associated delta-9-tetrahydrocannabinol 14 has been separated from second liquid phase 24 containing the preferentially associated one or more additional cannabinoids 16.

It should be understood that the term “separation,” as used herein, does not necessarily mean complete and absolute separation, but is used herein as to refer to the production of a liquid phase that is enriched in at least one of the components within the original mixture. In some embodiments, the “separation” of delta-9-tetrahydrocannabinol and another component can refer to the production of a liquid phase that is enriched in the delta-9-tetrahydrocannabinol relative to the amount of the delta-9-tetrahydrocannabinol and the other component in the original mixture (and, optionally, the production of a second liquid phase that is enriched in one or more additional cannabinoids that are not delta-9-tetrahydrocannabinol relative to the amount of the one or more additional cannabinoids and the delta-9-tetrahydrocannabinol in the original mixture).

In some embodiments, the separated first liquid phase (e.g., first liquid phase 22 shown in FIG. 1D) is enriched in delta-9-tetrahydrocannabinol relative to the amount of the delta-9-tetrahydrocannabinol in the original mixture (e.g., mixture 12 in FIG. 1A). For example, in some embodiments, the mole fraction of delta-9-tetrahydrocannabinol relative to all cannabinoids (including the delta-9-tetrahydrocannabinol and the one or more additional cannabinoids) in the first liquid phase may be at least 1.2 times, at least 1.5 times, at least 2 times, at least 3 times, at least 5 times, at least 10 times, at least 50 times, at least 100 times, or at least 1000 times (and/or up to 10⁴ times, up to 10⁵ times, or more) the mole fraction of delta-9-tetrahydrocannabinol relative to all cannabinoids in the original mixture. Combination of the above-referenced ranges are possible. Other ranges are also possible. As a non-limiting example, in some cases, the original mixture may contain 50 mol of delta-9-tetrahydrocannabinol and 100 mol total of all cannabinoids, which means the mole fraction of delta-9-tetrahydrocannabinol relative to all cannabinoids would be 0.5 (i.e., 50/100). After partitioning (via one or more stages), the first liquid phase may contain 45 mol of delta-9-tetrahydrocannabinol and 50 mol total of all cannabinoids, which means the mole fraction of delta-9-tetrahydrocannabinol relative to all cannabinoids in the first liquid phase would be 0.9 (i.e., 45/50). In this non-limiting example, the mole fraction of delta-9-tetrahydrocannabinol relative to all cannabinoids in the first liquid phase is 1.8 times the mole fraction of delta-9-tetrahydrocannabinol relative to all cannabinoids in the original mixture (because 0.9 divided by 0.5 is 1.8). In this example, the first liquid phase would be said to be enriched in delta-9-tetrahydrocannabinol relative to the original mixture because the mole fraction of delta-9-tetrahydrocannabinol relative to all cannabinoids in the first liquid phase is higher than the mole fraction of delta-9-tetrahydrocannabinol relative to all cannabinoids in the original mixture. As another non-limiting example, in some cases, the original mixture may contain 50 mol of delta-9-tetrahydrocannabinol and 100 mol total of all cannabinoids, which means the mole fraction of delta-9-tetrahydrocannabinol relative to all cannabinoids would be 0.5 (i.e., 50/100). After partitioning (via one or more stages), the second liquid phase may contain 5 mol of delta-9-tetrahydrocannabinol and 50 mol total of all cannabinoids, which means the mole fraction of delta-9-tetrahydrocannabinol relative to all cannabinoids in the second liquid phase would be 0.1 (i.e., 5/50). In this non-limiting example, the mole fraction of delta-9-tetrahydrocannabinol relative to all cannabinoids in the second liquid phase is 0.2 times the mole fraction of delta-9-tetrahydrocannabinol relative to all cannabinoids in the original mixture (because 0.1 divided by 0.5 is 0.2). In this example, the second liquid phase would not be said to be enriched in delta-9-tetrahydrocannabinol relative to the original mixture because the mole fraction of delta-9-tetrahydrocannabinol relative to all cannabinoids in the second liquid phase is lower than the mole fraction of delta-9-tetrahydrocannabinol relative to all cannabinoids in the original mixture.

In some embodiments, the mole fraction of delta-9-tetrahydrocannabinol relative to the total amount of delta-9-tetrahydrocannabinol and delta-8-tetrahydrocannabinol in the first liquid phase may be at least 1.2 times, at least 1.5 times, at least 2 times, at least 3 times, at least 5 times, at least 10 times, at least 50 times, at least 100 times, or at least 1000 times (and/or up to 10⁴ times, up to 10⁵ times, or more) the mole fraction of delta-9-tetrahydrocannabinol relative to the total amount of delta-9-tetrahydrocannabinol and delta-8-tetrahydrocannabinol in the original mixture. In some embodiments, the mole fraction of delta-9-tetrahydrocannabinol relative to the total amount of delta-9-tetrahydrocannabinol and cannabidiol in the first liquid phase may be at least 1.2 times, at least 1.5 times, at least 2 times, at least 3 times, at least 5 times, at least 10 times, at least 50 times, at least 100 times, or at least 1000 times (and/or up to 10⁴ times, up to 10⁵ times, or more) the mole fraction of delta-9-tetrahydrocannabinol relative to the total amount of delta-9-tetrahydrocannabinol and cannabidiol in the original mixture. In some embodiments, the separated second liquid phase (e.g., second liquid phase 24 shown in FIG. 1D) is enriched in one or more additional cannabinoids (that are not delta-9-tetrahydrocannabinol) relative to the amount of the cannabinoids in the original mixture (e.g., mixture 12 in FIG. 1A). For example, in some embodiments, the mole fraction of the one or more additional cannabinoids relative to all cannabinoids in the second liquid phase may be at least 1.2 times, at least 1.5 times, at least 2 times, at least 3 times, at least 5 times, at least 10 times, at least 50 times, at least 100 times, or at least 1000 times (and/or up to 10⁴ times, up to 10⁵ times, or more) the mole fraction of the one or more additional cannabinoids relative to all cannabinoids in the original mixture. In some embodiments, the mole fraction of delta-8-tetrahydrocannabinol relative to all cannabinoids in the second liquid phase may be at least 1.2 times, at least 1.5 times, at least 2 times, at least 3 times, at least 5 times, at least 10 times, at least 50 times, at least 100 times, or at least 1000 times (and/or up to 10⁴ times, up to 10⁵ times, or more) the mole fraction of delta-8-tetrahydrocannabinol relative to all cannabinoids in the original mixture. In some embodiments, the mole fraction of cannabidiol relative to all cannabinoids in the second liquid phase may be at least 1.2 times, at least 1.5 times, at least 2 times, at least 3 times, at least 5 times, at least 10 times, at least 50 times, at least 100 times, at least 1000 times, or at least 10⁵ times (and/or up to 10⁶ times, up to 10⁷ times, up to 10⁸ times, or more) the mole fraction of cannabidiol relative to all cannabinoids in the original mixture. Combination of the above-referenced ranges are possible. Other ranges are also possible.

In some embodiments, the mole fraction of delta-8-tetrahydrocannabinol relative to the total amount of delta-8-tetrahydrocannabinol and delta-9-tetrahydrocannabinol in the second liquid phase may be at least 1.2 times, at least 1.5 times, at least 2 times, at least 3 times, at least 5 times, at least 10 times, at least 50 times, at least 100 times, at least 1000 times, or at least 10⁵ times (and/or up to 10⁶ times, up to 10⁷ times, up to 10⁸ times, or more) the mole fraction of delta-8-tetrahydrocannabinol relative to the total amount of delta-8-tetrahydrocannabinol and delta-9-tetrahydrocannabinol in the original mixture. In some embodiments, the mole fraction of cannabidiol relative to the total amount of cannabidiol and delta-9-tetrahydrocannabinol in the second liquid phase may be at least 1.2 times, at least 1.5 times, at least 2 times, at least 3 times, at least 5 times, at least 10 times, at least 50 times, at least 100 times, or at least 1000 times (and/or up to 10⁴ times, up to 10⁵ times, or more) the mole fraction of cannabidiol relative to the total amount of cannabidiol and delta-9-tetrahydrocannabinol in the original mixture. Combination of the above-referenced ranges are possible. Other ranges are also possible.

In some embodiments, the method described herein may have a relatively high delta-9-tetrahydrocannabinol extraction efficiency. As used herein, the term “extraction efficiency” refers to the percentage of separated delta-9-tetrahydrocannabinol (e.g., delta-9-tetrahydrocannabinol that preferentially associates with the first liquid phase) relative to the total amount of delta-9-tetrahydrocannabinol in the mixture. For example, in some embodiments, the delta-9-tetrahydrocannabinol extraction efficiency may be greater than or equal to 80% (e.g., greater than or equal to 90%, greater than or equal to 95%, greater than or equal to 97%, greater than or equal to 98%, greater than or equal to 99%, greater than or equal to 99.5%, greater than or equal to 99.7%, greater than or equal to 99.9%, greater than or equal to 99.99%, or equal to 100%).

In some embodiments, the method may be performed as a multi-step separation process. FIG. 1E is a schematic illustration showing a multi-stage separation process, according to certain embodiments. As shown in FIG. 1E, an initial mixture 50 of a first phase and a second phase (comprising a first solute and a second solute) is subjected to separation process 51. Separation process 51 can be used to produce a first liquid phase 52, which can be enriched in the first solute relative to mixture 50. Separation process 51 can also produce a second liquid phase 53, which may be enriched in the second solute relative to mixture 50.

In FIG. 1E, first liquid phase 52 can be mixed with another liquid phase 54 (which can be the same liquid that is present in liquid phase 53, or another liquid) to produce mixture 55, which can be subjected to a second separation process 56. Second separation process 56 can be used to produce phase 57, which can be further enriched in the first solute relative to mixture 55 (and, in certain embodiments, mixture 50). Second separation process 56 can also be used to produce liquid phase 58, which can be enriched in the second solute relative to mixture 55 (and, in certain embodiments, mixture 50).

In FIG. 1E, liquid phase 57 can be mixed with another liquid phase 59 (which can be the same liquid that is present in liquid phase 53 and/or liquid phase 58, or another liquid) to produce mixture 60, which can be subjected to a third separation process 61. Third separation process 61 can be used to produce phase 62, which can be further enriched in the first solute relative to mixture 60 (and, in certain embodiments, mixture 55 and/or mixture 50). Third separation process 61 can also be used to produce liquid phase 63, which can be enriched in the second solute relative to mixture 60 (and, in certain embodiments, relative to mixture 55 and/or mixture 50).

In some embodiments, the method may be performed as a continuous extraction process. For example, in one set of embodiments, the method may be operated using a liquid-liquid continuous chromatography and/or a liquid-liquid extraction system. In some cases, the liquid-liquid extraction system is a multi-stage countercurrent liquid-liquid extraction system. The method described herein, when used with such systems, may advantageously reduce the number of stages necessary to achieve efficient extraction, reduce the amount of solvent needed for the extraction, and/or allow for continuous and selective extraction of a specific cannabinoid (e.g., delta-9-tetrahydrocannabinol) from a mixture.

In some embodiments, the association of the chemical species (e.g., the delta-9-tetrahydrocannabinol and the one or more additional cannabinoids) with their respective liquid phases (e.g., the first liquid phase, the second liquid phase) in the heterogeneous liquid mixture may correlate with the ability of the chemical species to selectively partition into the different liquid phases and the volumetric ratio between the different liquid phases. For example, in a biphasic heterogeneous liquid mixture comprising a first liquid phase and a second liquid phase, the association of a chemical species with the liquid phases may correlate with an extraction factor Y. For example, for chemical species i, the extraction factor Yᵢ may be expressed as: Yᵢ=Kᵢ·(V_{1st liquid phase}/V_{2nd liquid phase}), which is the product of the partition coefficient Kᵢ for species i and a ratio of a volume factor of the first liquid phase (V_{1st liquid phase}) and a volume factor of the second liquid phase (V_{2nd liquid phase}). In cases where the separation process is a batch separation process, the volume factor of each phase is the volume of that phase that is present (i.e., in a batch separation process, V_{1st liquid phase} corresponds to the volume of the first liquid phase that is present, and V_{2nd liquid phase} corresponds to the volume of the second liquid phase that is present). In cases where the separation process is one in which the first and second phases are flowed (e.g., in a continuous separation process), the volume factor of each phase is the volumetric flow rate of that phase (i.e., in a separation process in which the phases are flowing, V_{1st liquid phase} corresponds to the volumetric flow rate of the first liquid phase, and V_{2nd liquid phase} corresponds to the volumetric flow rate of the second liquid phase). As noted elsewhere herein, for chemical species i, the partition coefficient Kᵢ may be expressed as: Kᵢ=C_{i(1st liquid phase)}/C_{i(2nd liquid phase)}, which is a ratio of the concentration of chemical species i in the first liquid phase (C_{i, 1st liquid phase}) to the concentration of chemical species i in the second liquid phase (C_{i, 2st liquid phase}).

In the context of the present disclosure, chemical species i may refer to delta-9-tetrahydrocannabinol or any of the one or more additional cannabinoids. For example, in embodiments in which the mixture comprises the delta-9-tetrahydrocannabinol and the one or more additional cannabinoids, the delta-9-tetrahydrocannabinol may have an extraction factor Y_{d9,THC}, which, as described above, is expressed as a product of the partition coefficient K_{d9,THC} of the delta-9-tetrahydrocannabinol and the volume factor ratio (V_{1st liquid phase}/V_{2nd liquid phase}) between the first liquid phase and the second liquid phase, where K_{d9,THC} is expressed as a ratio of the concentration of delta-9-tetrahydrocannabinol in the first liquid phase to the concentration of delta-9-tetrahydrocannabinol in the second liquid phase (e.g., K_{d9,THC}=C_{d9,THC (1st liquid phase)}/C_{d9, THC (2nd liquid phase)}). Similarly, each of the one or more additional cannabinoids may individually have and/or collectively have an extraction factor Y_{cnbd, i}, which, as described above, is expressed as a product of the partition coefficient K_{cnbd, i}, of the one or more additional cannabinoids and the volume factor ratio (V_{1st liquid phase}/V_{2nd liquid phase}) between the first liquid phase and the second liquid phase, where K_{cnbd, i} is expressed as a ratio of the concentration of the one or more additional cannabinoids in the first liquid phase to the concentration of the one or more additional cannabinoids in the second liquid phase (e.g., K_{cnbd, i}=C_{cnbd, i (1st liquid phase)}/C_{cnbd, i (2nd liquid phase)}). For example, in embodiments in which the one or more additional cannabinoids comprise delta-8-tetrahydrocannabinol, an extraction factor Y_{d8,THC} for the delta-8-tetrahydrocannabinol may be expressed as a product of the partition coefficient K_{d8,THC} of the delta-8-tetrahydrocannabinol and the volume factor ratio (V_{1st liquid phase}/V_{2nd liquid phase}) between the first liquid phase and the second liquid phase, where K_{d8,THC} is expressed as a ratio of the concentration of delta-8-tetrahydrocannabinol in the first liquid phase to the concentration of delta-8-tetrahydrocannabinol in the second liquid phase (e.g., K_{d8,THC}=C_{d8, THC (1st liquid phase)}/C_{d8, THC (2nd liquid phase)}). As another example, in embodiments in which the one or more additional cannabinoids comprise cannabidiol, an extraction factor Y_{CBD} for the cannabidiol may be expressed as a product of the partition coefficient K_{CBD} of the cannabidiol and the volumetric ratio (V_{1st liquid phase}/V_{2nd liquid phase}) between the first liquid phase and the second liquid phase, where K_{CBD} is expressed as a ratio of the concentration of cannabidiol in the first liquid phase to the concentration of cannabidiol in the second liquid phase (e.g., K_{CBD}=C_{CBD (1st liquid phase)}/C_{CBD (2nd liquid phase)}).

In some embodiments, it may be advantageous to select a heterogeneous liquid mixture having a particular combination of extraction factors (e.g., Y_{d9,THC}, Y_{cnbd,i}), e.g., such as an extraction factor Y_{d9,THC} of the delta-9-tetrahydrocannabinol of greater than 1 and an extraction factor Y_{cnbd,i} (e.g., Y_{d8,THC}, Y_{CBD}, etc.) of the one or more additional cannabinoids of less than 1, or vice versa. Without wishing to be bound by any particular theory, it is hypothesized that such a particular combination of extraction factors may lead to efficient separation of one or more chemical species, e.g., such as the separation of delta-9-tetrahydrocannabinol from the one or more additional cannabinoids. For example, in some embodiments, the delta-9-tetrahydrocannabinol may have an extraction factor Y_{d9,THC} of greater than 1, greater than or equal to 1.05, greater than or equal to 1.1, greater than or equal to 1.15, greater than or equal to 1.2, greater than or equal to 1.25, greater than or equal to 1.3, greater than or equal to 1.4, greater than or equal to 1.6, greater than or equal to 1.8, greater than or equal to 2, or greater (and/or, in some embodiments, up to 2.5, up to 3, up to 4, up to 5, up to 6, up to 8, or up to 10, or more). Combinations of the above-referenced ranges are possible (e.g., greater than 1 and up to 10). Other ranges are also possible. Additionally, in some embodiments, the one or more additional cannabinoids (e.g., delta-8-tetrahydrocannabinol, cannabidiol, etc.) may have an extraction factor Y_{cnbd,i} (e.g., Y_{d8,THC}, Y_{CBD}) of less than 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, less than or equal to 0.2, less than or equal to 0.1, or less (and/or down to 0.01, down to 0.001, or less). Combinations of the above-referenced ranges are possible (e.g., less than 1 and down to 0.001). Other ranges are also possible.

In embodiments in which a multi-stage liquid-liquid extraction system is employed for separating the delta-9-tetrahydrocannabinol from the one or more additional cannabinoids, the delta-9-tetrahydrocannabinol and the one or more additional cannabinoids in one or more of the plurality of stages in the extraction system may each have an extraction factor in one or more of ranges described above.

The method for separating the delta-9-tetrahydrocannabinol from the one or more additional cannabinoids may be performed at any of a variety of operating conditions. In some embodiments, the method may be performed at an operating pressure of at least 0.6 atmospheres absolute, at least 0.8 atmospheres absolute, at least 0.9 atmospheres absolute, at least 0.95 atmospheres absolute, at least 0.98 atmospheres absolute and/or less than or equal to 2.0 atmospheres absolute, less than or equal to 1.5 atmospheres absolute, less than or equal to 1.3 atmospheres absolute, less than or equal to 1.2 atmospheres absolute, less than or equal to 1.1 atmospheres absolute, less than or equal to 1.05 atmospheres absolute, and/or less than or equal to 1.02 atmospheres absolute. Combinations of the above-reference ranges are possible (e.g., at least 0.6 atmospheres absolute and less than or equal to 2.0 atmospheres absolute). Other ranges are also possible.

In some embodiments, the method may be performed at an operating temperature of greater than or equal to 5° C., greater than or equal to 10° C., greater than or equal to 15° C., greater than or equal to 20° C., greater than or equal to 30° C., greater than or equal to 40° C., or greater than or equal to 50° C. In some embodiments, the method may be performed at an operating temperature of less than or equal to 60° C., less than or equal to 50° C., less than or equal to 40° C., less than or equal to 30° C., less than or equal to 20° C., less than or equal to 15° C., or less than or equal to 10° C. Combinations of the above-referenced ranges are possible (e.g., greater than or equal to 5° C. and less than or equal to 60° C.). Other ranges are also possible.

In some embodiments, the method described herein may be employed to separate delta-9-tetrahydrocannabinol from delta-8-tetrahydrocannabinol in a mixture. In some embodiments, to separate the delta-9-tetrahydrocannabinol from the delta-8-tetrahydrocannabinol in the mixture, the mixture may be first exposed to the heterogeneous liquid mixture, e.g., as shown in FIG. 1A. The heterogeneous liquid mixture may comprise any type of first liquid phase and second liquid phase described herein. Upon exposing the mixture comprising delta-9-tetrahydrocannabinol and delta-8-tetrahydrocannabinol to the heterogeneous liquid mixture, the delta-9-tetrahydrocannabinol may preferentially associate with the first liquid phase and the delta-8-tetrahydrocannabinol may preferentially associate with the second liquid phase, e.g., as shown in FIGS. 1B-1C. In some embodiments, the heterogeneous liquid mixture may be selected based on the partition coefficients of the delta-9-tetrahydrocannabinol and the delta-8-tetrahydrocannabinol. The delta-9-tetrahydrocannabinol may have a partition coefficient K_{d9,THC} in one or more ranges described elsewhere herein. Similarly, the delta-8-tetrahydrocannabinol may have a partition coefficient K_{d8,THC} in one or more ranges described above with respect to the partition coefficient K_{cnbd,i} of the one or more additional cannabinoids. In some cases, the delta-9-tetrahydrocannabinol and the delta-8-tetrahydrocannabinol may have a ratio of K_{d9,THC} to K_{d8,THC} in one or more of the ranges described above with respect to K_{d9,THC} to K_{cnbd, i}.

In some embodiments, the method described herein may be employed to separate delta-9-tetrahydrocannabinol from cannabidiol in a mixture. In some embodiments, to separate the delta-9-tetrahydrocannabinol from the cannabidiol in the mixture, the mixture may be first exposed to the heterogeneous liquid mixture, e.g., as shown in FIG. 1A. The heterogeneous liquid mixture may comprise any type of first liquid phase and second liquid phase described herein. Upon exposing the mixture comprising delta-9-tetrahydrocannabinol and cannabidiol to the heterogeneous liquid mixture, the delta-9-tetrahydrocannabinol may preferentially associate with the first liquid phase and the cannabidiol may preferentially associate with the second liquid phase, e.g., as shown in FIGS. 1B-1C. In some embodiments, the heterogeneous liquid mixture may be selected based on the partition coefficients of the delta-9-tetrahydrocannabinol and the cannabidiol. The delta-9-tetrahydrocannabinol may have a partition coefficient K_{d9,THC} in one or more ranges described elsewhere herein. The cannabidiol may have a partition coefficient K_{CBD} in one or more ranges described above with respect to the partition coefficient K_{cnbd, i} of the one or more additional cannabinoids. In some cases, the delta-9-tetrahydrocannabinol and the cannabidiol may have a ratio of K_{d9,THC} to K_{CBD} in one or more of the ranges described above with respect to K_{d9,THC} to K_{cnbd, i}.

Certain embodiments related to an ingestible composition. In one set of embodiments, the ingestible composition comprises delta-9-tetrahydrocannabinol and, optionally, one or more additional cannabinoids. The one or more additional cannabinoids may include any of a variety of additional cannabinoids described elsewhere herein. For example, the one or more additional cannabinoids may comprise delta-8-tetrahydrocannabinol. Alternatively or additionally, the one or more additional cannabinoids may comprise cannabidiol.

The ingestible composition may have any of a variety of appropriate volumes. In some embodiments, the ingestible composition may have a volume of at least 1 mm³ (e.g., at least 2 mm³, at least 5 mm³, at least 7 mm³, or at least 2 mm³). In some embodiments, the ingestible composition may have a volume of up to 20 mm³ (e.g., up to 40 mm³, up to 60 mm³, up to least 80 mm³, or up to 100 mm³). Combinations of the above-referenced ranges are possible (e.g., at least 2 mm³ and up to 100 mm³). Other ranges are also possible.

The ingestible composition may comprise the delta-9-tetrahydrocannabinol in any of a variety of appropriate amounts. In some embodiments, the amount of delta-9-tetrahydrocannabinol within the composition may be at least 0.01 wt %, at least 0.1 wt %, at least 1 wt %, at least 10 wt %, at least 20 wt %, at least 50 wt %, at least 75 wt %, at least 90 wt %, or more. In some embodiments, the amount of delta-9-tetrahydrocannabinol within the composition may be up to 2000 mg, up to 2500 mg, up to 3000 mg, up to 4000 mg, or more. Combinations of the above-referenced ranges are also possible (e.g., at least 0.01 mg and up to 4000 mg). Other ranges are also possible.

The delta-9-tetrahydrocannabinol and the one or more additional cannabinoids (e.g., delta-8-tetrahydrocannabinol, cannabidiol, etc.) may be present in the composition in any appropriate molar ratio. For example, a molar ratio of the delta-9-tetrahydrocannabinol to the one or more cannabinoids within the ingestible composition may be greater than or equal to 3:1 (e.g., greater than or equal to 5:1, greater than or equal to 9:1, greater than or equal to 95:5, greater than or equal to 97:3, greater than or equal to 99:1, or greater than or equal to 99.9:0.1).

The present disclosure is also related to the continuous multi-stage separation of chemical species using multiple liquid phases, as well as related systems and articles. In certain embodiments, the continuous multi-stage separation process can be used to separate delta-9-tetrahydrocannabinol (e.g., as a first solute) from delta-8-tetrahydrocannabinol and/or one or more other cannabinoids (e.g., as a second solute).

Certain aspects of the present disclosure are directed to the discovery that the use of multi-stage liquid-liquid countercurrent chromatographic separator systems can allow for highly efficient and targeted separation of a chemical species (e.g., a first solute) from one or more additional chemical species (e.g., a second solute) in a feed liquid stream. Certain embodiments are related to the discovery that the use of two mobile phases, e.g., such as a first liquid phase and a second liquid phase that is distinct from (e.g., immiscible with) the first liquid phase, can provide, in certain instances, one or more of a variety of operational advantages compared to conventional systems. Such operational advantages include, but are not limited to, a high throughput continuous extraction process, recycling of solvent(s), ease of scalability, a high degree of separation, and/or a high extraction efficiency associated with a target chemical species. Some embodiments are related to the discovery that effective separation of a specific chemical species can be achieved by using liquids that provide different partition coefficients of the chemical species and the one or more additional chemical species in the two mobile phases. It has also been recognized, within the context of the present disclosure, that the systems and methods described herein can be advantageously employed in the purification of any of a variety of chemical species. Compared to conventional systems and methods, in accordance with certain embodiments, systems and methods described herein can allow one to effectively target a specific chemical species, use less extraction solvent(s) and/or extraction stages, and/or reduce overall operational costs associated with the separation process.

In some embodiments, liquid-liquid chromatographic separator systems and related methods are described. The separator systems and related methods can be employed for separating a first solute from a second solute in a feed liquid stream using two mobile phases (e.g., a first liquid phase and a second liquid phase distinct from the first liquid phase) based on the ability of the two solutes to partition into different mobile phases to a different degree and the ability of the two mobile phases to phase separate. The separator systems can comprise, in some embodiments, a series of liquid-liquid chromatographic separator stages, each of which is capable of phase separating a mixed liquid stream comprising the two mobile phases into two liquid streams, e.g., one comprising predominantly one mobile phase having a mole fraction of the first solute relative to the sum of the first solute and the second solute that is larger than a mole fraction of the first solute relative to the sum of the first solute and the second solute in the mixed liquid stream received by the separator stage, and one comprising predominantly the other mobile phase having a mole fraction of the second solute relative to the sum of the first solute and the second solute that is larger than a mole fraction of the second solute relative to the sum of the first solute and the second solute in the mixed liquid stream received by the separator stage.

While many of the embodiments described herein include a first solute and a second solute, it should be understood that more than two solutes can be present, in certain embodiments.

As noted above, in association with certain of the embodiments described herein, certain liquids are said to be “enriched” in a first solute or a second solute, relative to another liquid. In this context, a first liquid is said to be “enriched” in the first solute relative to a second liquid if the mole fraction of the first solute relative to the sum of the first solute and the second solute in the first liquid is higher than the mole fraction of the first solute relative to the sum of the first solute and the second solute in the second liquid. Similarly, a first liquid is said to be “enriched” in the second solute relative to a second liquid if a mole fraction of the second solute relative to the sum of the first solute and the second solute in the first liquid is higher than a mole fraction of the second solute relative to the sum of the first solute and the second solute in the second liquid. In some instances in which a first liquid is enriched in a solute relative to a second liquid, it is particularly advantageous if the concentration of the solute in the first liquid is higher than the concentration of that solute in the second liquid. For example, in some embodiments, it is particularly advantageous if the separator stage (e.g., each separator stage within the multi-stage system) produces (1) a first liquid that has a higher concentration of first solute than the concentration of the first solute in the stream that is input to the separator stage and (2) a second liquid that has a higher concentration of second solute than the concentration of the second solute in the stream that is input to the separator stage.

Certain of the methods disclosed herein can involve, in some embodiments, transporting a feed liquid stream comprising two solutes and at least one of the two mobile phases (and, in some cases, both mobile phases) into the separator system described herein. The methods can, in certain embodiments, allow for separation of the two solutes via differential partitioning of the two solutes into different mobile phases. In some such embodiments, subsequent phase separation of the mobile phases (e.g., immiscible mobile phases) can produce two liquid streams, e.g., one stream comprising predominantly one mobile phase having a mole fraction of the first solute relative to the sum of the first solute and the second solute that is larger than a mole fraction of the first solute relative to the sum of the first solute and the second solute in the feed liquid stream, and a second stream comprising predominantly the other mobile phase having a mole fraction of the second solute relative to the sum of the first solute and the second solute that is larger than a mole fraction of the second solute relative to the sum of the first solute and the second solute in the feed liquid stream.

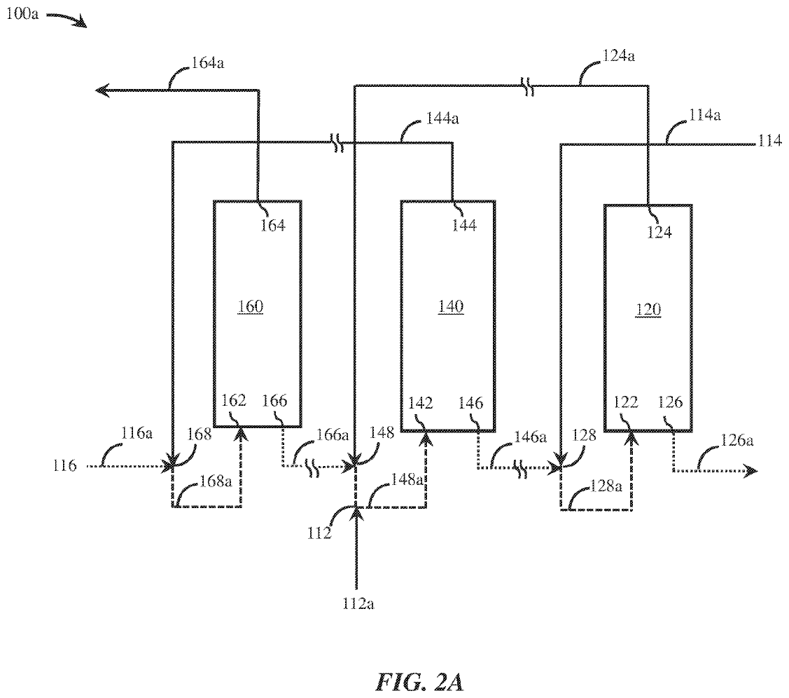

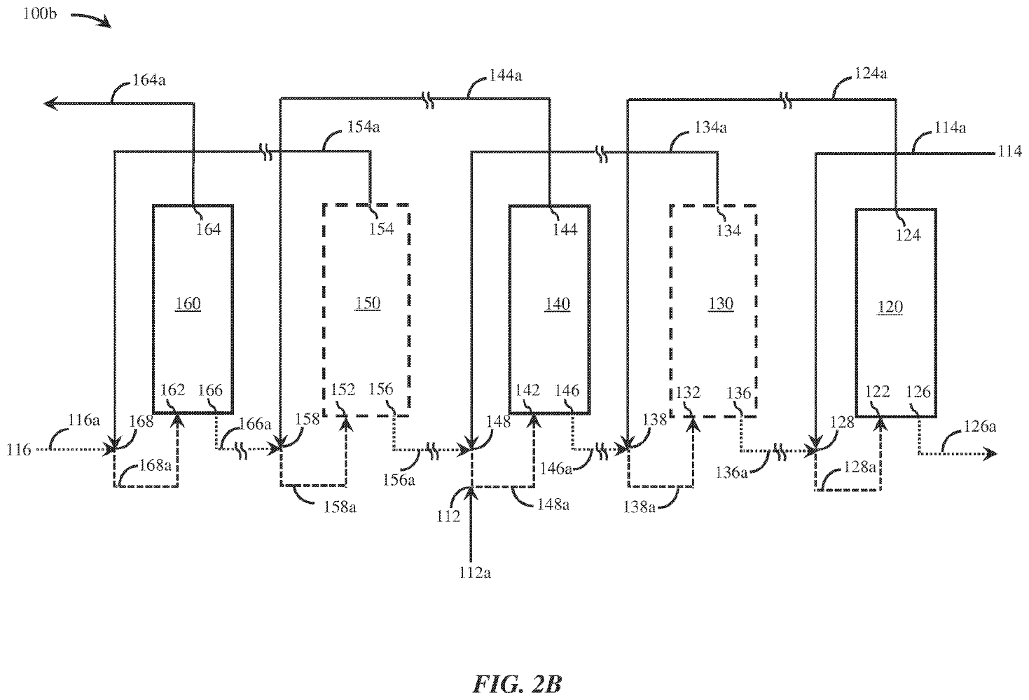

FIGS. 2A-2B are schematic illustrations of non-limiting embodiments of liquid-liquid chromatographic separator systems comprising a plurality of separator stages. The system in FIG. 2A depicts three separator stages (and can include more separator stages), while the system in FIG. 2B depicts five separator stages (and can include more separator stages). These figures are referred to throughout the disclosure below.

In some embodiments, a liquid-liquid chromatographic separator system is described. The liquid-liquid chromatographic separator system, in certain embodiments, is a multi-stage liquid-liquid chromatographic separator system comprising a plurality of separator stages (e.g., liquid-liquid phase chromatographic separator stages). The liquid-liquid chromatographic separator system may comprise any of a variety of appropriate numbers of separator stages, including, but not limited to, three or more stages, four or more stages, five or more stages, six or more stages, eight or more stages, ten or more stages, twenty or more stages, thirty or more stages, or fifty or more stage stages (and/or up to 100 stages, up to 500 stages, up to 1000 stages, or more).

In some embodiments, the plurality of separator stages within the liquid-liquid chromatographic separator system are arranged in series with one another from a first separator stage to a last separator stage and fluidically connected to one another in succession. One or more intermediate separator stages may, in certain embodiments, be arranged between and fluidically connected to the first separator stage and the last separator stage. Any appropriate number of intermediate separator stages may be present between the first separator stage and the last separator stage. For example, in some embodiments, the system comprises at least 1, at least 2, at least 3, at least 4, at least 5, at least 10, at least 20, at least 30, at least 40, or more (and/or up to 50, up to 100, up to 500, up to 1000, or more) intermediate separator stages.

FIGS. 2A-2B are schematic illustrations of non-limiting embodiments of liquid-liquid chromatographic separator systems comprising a plurality of separator stages.

As shown in FIG. 2A, liquid-liquid chromatographic separator system 100a comprises three separator stages (120, 140, and 160) fluidically connected in succession. While FIG. 2A shows three separator stages present, additional separator stages can also be present (indicated by the broken lines shown in streams 124a, 144a, 146a, and 166a, described in more detail below).

The three separator stages may be arranged in series with one another from first separator stage 120 to last separator stage 160, with intermediate separator stage 140 positioned between first separator stage 120 and last separator stage 160. First separator stage 120 may be fluidically connected to intermediate separator stage 140, which may be fluidically connected to last separator stage 160. For example, as shown in FIG. 2A, stage 120 is fluidically connected, in series, to stage 140 via streams 124a and 148a. Stages 120 and 140 are also fluidically connected in series via streams 146a and 128a. Also as shown in FIG. 2A, stage 140 is fluidically connected, in series, to stage 160 via streams 144a and 168a. Stages 140 and 160 are also fluidically connected in series via streams 166a and 148a.

While FIG. 2A shows a single intermediate separator stage between the first separator stage and the last separator stage, it should be understood that not all embodiments described herein are so limiting, and in other embodiments, additional intermediate separator stages may present between the first separator stage and the last separator stage. For example, as shown in FIG. 2B, additional separator stage(s) (e.g., first additional separator stage 130) may be present between first separator stage 120 and intermediate separator stage 140 and/or additional separator stage(s) (e.g., second additional intermediate separator stage 150) may present between intermediate separator stage 140 and last separator stage 160. It should be understood that additional separator stages may also be present between any of the above-referenced intermediate separator stages illustrated in FIGS. 2A-2B.

It should be understood that fluidic connectivity between the various separator stages (e.g., between first separator stage 120 and intermediate separator stage 140, between intermediate separator stage 140 and last separator stage 160, etc.) and/or between fluid sources and separator stages illustrated in FIG. 2A may be either a direct fluidic connectivity or an indirect fluidic connectivity. As used herein, “direct fluidic connectivity” between a first stage and a second stage is said to exist when a stream passes from the first stage to the second stage without passing through another stage. Similarly, “direct fluidic connectivity” between a source and a stage is said to exist when a stream passes from the source to the stage without passing through another stage. Also, as used herein, “indirect fluidic connectivity” between a first stage and a second stage is said to exist when a stream passes from the first stage to the second stage but first passes through another stage. Similarly, as used herein, “indirect fluidic connectivity” between a source and a stage is said to exist when a stream passes from the source to the stage but first passes through another stage. To illustrate, referring to the fluidic connectivity between intermediate separator stage 140 and first separator stage 120, in embodiments in which no additional intermediate separator stages are present between intermediate separator stage 140 and first separator stage 120, the fluidic connectivity between intermediate separator stage 140 and first separator stage 120 is direct (and the two stages are said to be directly fluidically connected to each other). For example, as shown in FIG. 2A, when the fluidic connectivity is a direct fluidic connectivity, a liquid (e.g., liquid 124a) exiting first separator stage 120 may be directly passed to intermediate separator stage 140 without first passing through another separator stage. Conversely, in embodiments in which one or more additional intermediate separator stages are present between first separator stage 120 and intermediate separator stage 140 such that stream 124a first passes through the additional intermediate stage before being transported from stage 120 to stage 140, the fluidic connectivity between intermediate separator stage 140 and first separator stage 120 via stream 124a is an indirect fluidic connectivity. A non-limiting example of such an indirect fluidic connectivity is illustrated in FIG. 2B. As shown in FIG. 2B, the fluidic connectivities between first separator stage 120 and intermediate separator stage 140 (via both the pathway that includes streams 124a, 138a, 134a, and 148a as well as the pathway that includes streams 146a, 138a, 136a, and 128a) are both indirect because each of these pathways includes stage 130 between stage 120 and stage 140.

While FIGS. 2A-2B illustrate non-limiting embodiments of a liquid-liquid chromatographic separator system comprising more than two separator stages (e.g., three or more stages, five or more stages), it should be understood that not all embodiments described herein are so limiting, and in other embodiments, the liquid-liquid chromatographic separator system may also be a two-stage separator system, e.g., such as comprising solely a first separator stage and a last separator stage, without any intermediate separator stages in between.

In some embodiments, the liquid-liquid chromatographic separator system comprises a feed liquid inlet configured to receive a feed liquid stream. The feed liquid stream, in certain embodiments, comprises a mixture of a first solute and a second solute. The feed liquid stream may optionally comprise a liquid carrier in which the first solute and the second solute are suspended and/or solubilized. FIGS. 2A-2B illustrate non-limiting examples of one such set of embodiments. As shown in FIGS. 2A-2B, liquid-liquid chromatographic separator systems 100a and 100b comprise feed liquid inlet 112. Feed liquid inlet 112 may be configured to receive feed liquid stream 112a comprising a first solute and a second solute. In accordance with certain embodiments, as discussed in more detail below, the first solute may have a higher affinity for a first liquid phase than the second solute, while the second solute may have a higher affinity for a second liquid phase distinct from (e.g., immiscible with) the first liquid phase than the first solute. Additionally or alternatively, in certain embodiments, the first liquid phase may have a higher affinity for a first solute than the second liquid phase, while the second liquid phase may have a higher affinity for a second solute than the first liquid phase.

The feed liquid inlet may be present in any of a variety of appropriate locations in the liquid-liquid chromatographic separator system. For example, in one set of embodiments, the feed liquid inlet may be positioned such that feed liquid stream feeds into one of the one or more intermediate separator stages before passing through the first separator stage or the last separator stage. For example, as shown in FIGS. 2A-2B, feed liquid inlet 112 may be positioned such feed liquid stream 112a feeds into intermediate separator stage 140 before passing through first separator stage 120 or last separator stage 160.

While FIG. 2A illustrates a non-limiting embodiment of a feed liquid inlet positioned such that feed liquid stream feeds directly into a particular intermediate separator stage (e.g., intermediate separator stage 140), it should be understood that not all embodiments described herein are so limiting, and in other embodiments, the feed liquid inlet may be positioned such that the feed liquid stream feeds into any intermediate separator stage(s) (e.g., such as additional intermediate separator stage 130 and 150 shown in FIG. 2B).

In some embodiments, the liquid-liquid chromatographic separator system is fluidically connected to sources containing two or more distinct liquid phases, e.g., a source containing a first liquid phase and a source containing a second liquid phase. The first liquid phase and the second liquid phase may be, in some embodiments, immiscible with each other. The two or more distinct liquid phases may have a low mutual solubility with each other. For example, in some embodiments, the two or more distinct liquid phases have a mutual solubility of less than or equal to 200 mg/mL, less than or equal to 10 mg/mL, less than or equal to 1 mg/mL, less than or equal to 0.1 mg/mL, less than or equal to 0.001 mg/mL, less than or equal to 0.0001 mg/mL, or less than or equal to 0.00001 mg/mL (and/or, as little as 0.000001 mg/mL, as little as 0.0000001 mg/mL, or less) at the temperature at which the separation process is carried out. In some embodiments, the two or more distinct liquid phases have a mutual solubility of less than or equal to 200 mg/mL, less than or equal to 10 mg/mL, less than or equal to 1 mg/mL, less than or equal to 0.1 mg/mL, less than or equal to 0.001 mg/mL, less than or equal to 0.0001 mg/mL, or less than or equal to 0.00001 mg/mL (and/or, as little as 0.000001 mg/mL, as little as 0.0000001 mg/mL, or less) at 20° C. In some embodiments, the first liquid phase and the second liquid phase have mutual solubilities falling within any of the ranges outlined above.

FIGS. 2A-2B illustrate non-limiting examples of one such set of embodiments. As shown in FIGS. 2A-2B, each of liquid-liquid chromatographic separator systems 100a and 100b is fluidically connected to source 114 containing a first liquid phase and source 116 containing a second liquid phase distinct from (e.g., immiscible with) the first liquid phase.

In some embodiments, a source containing the first liquid phase may be fluidically connected to (e.g., directly fluidically connected to) a first liquid inlet of the first separator stage, and a source containing the second liquid phase may be fluidically connected to (e.g., directly fluidically connected to) a last liquid inlet of the last separator stage. The first liquid inlet, in certain embodiments, is configured to receive a first liquid phase from a source containing the first liquid phase, while the last liquid inlet is configured to receive a second liquid phase that is distinct from (e.g., immiscible with) the first liquid phase from the source containing the second liquid phase. FIGS. 2A-2B illustrate non-limiting examples of one such set of embodiments. As shown in FIGS. 2A-2B, source 114 containing the first liquid phase is fluidically connected to first liquid inlet 122 of first separator stage 120, such that first liquid inlet 122 is configured to receive first liquid phase 114a from source 114. Additionally, in FIGS. 2A-2B, source 116 containing the second liquid phase is fluidically connected to last liquid inlet 162 of last separator stage 160, such that last liquid inlet 162 is configured to receive second liquid phase 116a from source 116.

In some embodiments, each of the first liquid phase and the second liquid phase may have different affinities for the first solute from the feed liquid stream and the second solute from the feed liquid stream. For example, in certain embodiments, compared to the second liquid phase, the first liquid phase may have a higher affinity for (e.g., a higher solubility for) the first solute than for the second solute, e.g., such that the first solute has the ability to preferentially associate with the first liquid phase. Conversely, compared to the first liquid phase, the second liquid phase may have a higher affinity for (e.g., a higher solubility for) the second solute than for the first solute, e.g., such that the second solute has the ability to preferentially associate with the second liquid phase. As described in more detail below, the preferential association of the solutes with their respective liquid phases (e.g., the first solute with the first liquid phase, the second solute with the second liquid phase) may be related to the ability of the solute to selectively partition into the different liquid phases. The first solute and the second solute may have any of a variety of partition coefficients relative to the first and second liquid phases, as described in more detail below and elsewhere herein.

In some embodiments, each of the plurality of separator stages within the liquid-liquid chromatographic separator system comprises a liquid inlet and two liquid outlets. For example, each of the first separator stage, last separator stage, and the one or more intermediate separator stage(s) may comprise a liquid inlet and two liquid outlets. As described in more detail below, each separator stage may be configured to receive a mixed liquid stream comprising the two distinct liquid phases (e.g., the first liquid phase and the second liquid phase) and two solutes (e.g., the first solute and the second solute from the feed liquid stream) via the liquid inlet. The separator stage may be configured to separate the mixed liquid stream into two liquids, e.g., one comprising predominantly the first liquid phase having a mole fraction of the first solute relative to the sum of the first solute and the second solute that is larger than a mole fraction of the first solute relative to the sum of the first solute and the second solute in the mixed liquid stream received by the separator stage, and the other comprising predominantly the second liquid phase having a mole fraction of the second solute relative to the sum of the first solute and the second solute that is larger than a mole fraction of the second solute relative to the sum of the first solute and the second solute in the mixed liquid stream received by the separator stage, and output the two liquids via the two liquid outlets.

Any of a variety of suitable separation devices and/or components may be employed in the separator stage to separate the two liquid phases. For example, in one set of embodiments, at least one of the plurality of separator stages comprises a porous medium-based fluidic separator (e.g., a membrane-based separator). As described in more detail below, the porous medium-based separator may be employed to separate a mixed liquid stream comprising two liquid phases into two separated liquids (e.g., two separated liquid streams) based on a polarity difference between the two liquid phases. Additional examples of suitable separation devices are described in more detail below.

As used herein, something (e.g., a liquid, a stream, a container, etc.) is said to “predominantly” contain a first liquid phase if the first liquid phase makes up at least 90 wt % (or, in some embodiments, at least 95 wt %, at least 98 wt %, at least 99 wt %, at least 99.5 wt %, at least 99.9 wt %, at least 99.99 wt %, or at least 99.999 wt % (and/or, up to 99.99999 wt %, or up to 100 wt %) of the total mass of the first liquid phase and the second liquid phase. Combinations of the above-referenced ranges are possible (e.g., at least 90 wt % and up to 100 wt. %). Other ranges are also possible.

Similarly, as used herein, something (e.g., a liquid, a stream, a container, etc.) is said to “predominantly” contain a second liquid phase if the second liquid phase makes up at least 90 wt % (or, in some embodiments, at least 95 wt %, at least 98 wt %, at least 99 wt %, at least 99.5 wt %, at least 99.9 wt %, at least 99.99 wt %, or at least 99.999 wt % (and/or, up to 99.99999 wt %, or up to 100 wt %) of the total mass of the first liquid phase and the second liquid phase. Combinations of the above-referenced ranges are possible (e.g., at least 90 wt % and up to 100 wt. %). Other ranges are also possible.

FIGS. 2A-2B illustrate non-limiting examples of embodiments in which separator stages take in a mixture of liquid phases and produce two separated liquid streams. For example, as shown in FIG. 2A, in liquid-liquid chromatographic separator system 100a, each of the plurality of separator stages (e.g., first separator stage 120, intermediate separator stage 140, last separator stage 160, etc.) comprises a liquid inlet and two liquid outlets. In FIGS. 2A-2B, each of the separator stages may be configured to receive a mixed liquid stream (e.g., mixed liquid stream 128a into stage 120, mixed liquid stream 148a into stage 140, and mixed liquid stream 168a into stage 160) comprising two distinct liquid phases (e.g., the first liquid phase and the second liquid phase) and two solutes (e.g., the first solute and the second solute) via the liquid inlet (e.g., via inlet 122, 142, and 162, respectively). The mixed liquid streams are indicated by dashed lines in FIGS. 2A-2B. In FIGS. 2A-2B, each of the separator stages can be configured to separate the two liquid phases from each other into two separate streams, and output the two streams via the two liquid outlets (e.g., outlets 124 and 126 in stage 120, outlets 144 and 146 in stage 140, and outlets 164 and 166 in stage 160). In FIGS. 2A-2B, each of the separator stages (e.g., separator stages 120, 140, 160) may output a liquid (e.g., liquid 126a, 146a, 166a) comprising predominantly the second liquid phase (with little, if any, of the first liquid phase) via one liquid outlet (e.g., outlet 126, 146, 166). In FIGS. 2A-2B, the streams that contain predominantly the second liquid phase are shown in dotted lines. In FIGS. 2A-2B, each of the separator stages (e.g., separator stages 120, 140, 160) may output another liquid (e.g., liquid 124a, 144a, 164a) comprising predominantly the first liquid phase (with little, if any, of the second liquid phase) via another liquid outlet (e.g., outlet 124, 144, 164). In FIGS. 2A-2B, the streams that contain predominantly the first liquid phase are shown in solid lines.

In embodiments in which the liquid-liquid chromatographic separator system comprises additional separator stages (e.g., such as first additional intermediate separator stage 130, second additional intermediate separator stage 150, etc., as shown in FIG. 2B), each of the additional separator stages may have a similar or identical structure and/or components as the separator stages described above, e.g., such as having a liquid inlet and two liquid outlets, etc. For example, each of the additional intermediate separator stages (e.g., separator stage 130, 150, etc.) may be configured to receive a mixed liquid stream (e.g., mixed liquid stream 138a for stage 130 and mixed liquid stream 158a for stage 150) comprising two distinct liquid phases (e.g., a first liquid phase and a second liquid phase) via the respective liquid inlet (e.g., inlet 132 for stage 130 and inlet 152 for stage 150). In FIG. 2B, each of the additional intermediate separator stages may separate the two distinct liquid phases from each other into two liquid streams, and output the two liquid streams via the two liquid outlets (e.g., outlets 134 and 136 for stage 130, and outlets 154 and 156 for stage 150, etc.). Each of the additional intermediate separator stages may output a liquid (e.g., liquid 136a or 156a) that comprises predominantly the second liquid phase (with little, if any, of the first liquid phase) via one liquid outlet (e.g., outlet 136 or 156), and output another liquid (e.g., liquid 134a or 154a) that comprises predominantly the first liquid phase (with little, if any, of the second liquid phase) via another liquid outlet (e.g., outlet 134 or 154).

Specifics of each of the separator stages and the associated inlets and outlets shown in FIGS. 2A-2B are described in more detail below.

In some embodiments, the first separator stage comprises a first liquid inlet. The first liquid inlet, in certain embodiments, may be configured to receive liquid comprising at least a portion of the first solute from the feed liquid stream and at least a portion of the second solute from the feed liquid stream. As described in more detail below, the liquid comprising at least a portion of the first solute and at least a portion of the second solute received by the first liquid inlet may be a mixed liquid stream comprising a first liquid phase and a second liquid phase distinct from the first liquid phase. FIGS. 2A-2B illustrate examples of one such set of embodiments. As shown in FIGS. 2A-2B, first separator stage 120 comprises first liquid inlet 122 configured to receive liquid 128a comprising at least a portion of the first solute from feed liquid stream 112a and at least a portion of the second solute from feed liquid stream 112a. Liquid 128a comprising at least a portion of the first solute and at least a portion of the second solute received by first liquid inlet 122 may be a mixed liquid stream comprising a first liquid phase and a second liquid phase distinct from the first liquid phase.

As used herein, the phrase “at least a portion” (e.g., whether referring to a liquid, a stream, a solute, or any other item) means some or all. In some embodiments, “at least a portion” of an item (e.g., a liquid, a stream, a solute, etc.) means at least 0.01 wt. %, at least 0.05 wt. %, at least 0.1 wt. %, at least 0.5 wt. %, at least 1 wt. %, at least 5 wt. %, at least 10 wt. %, at least 20 wt. %, at least 30 wt. %, at least 40 wt. %, at least 50 wt. %, at least 60 wt. %, at least 70 wt. %, at least 80 wt. %, at least 90 wt. %, at least 95 wt. %, at least 99 wt. %, at least 99.9 wt. %, or up to 100 wt. %.

For example, in some embodiments, a liquid comprising “at least a portion” of the first solute from the feed liquid stream contains at least 0.01 wt. %, at least 0.05 wt. %, at least 0.1 wt. %, at least 0.5 wt. %, at least 1 wt. %, at least 5 wt. %, at least 10 wt. %, at least 20 wt. %, at least 30 wt. %, at least 40 wt. %, at least 50 wt. %, at least 60 wt. %, at least 70 wt. %, at least 80 wt. %, at least 90 wt. %, at least 95 wt. %, at least 99 wt. %, at least 99.9 wt. %, or up to 100 wt. % of the first solute from the feed liquid stream. As another example, in some embodiments, a liquid comprising “at least a portion” of the second solute from the feed liquid stream contains at least 0.01 wt. %, at least 0.05 wt. %, at least 0.1 wt. %, at least 0.5 wt. %, at least 1 wt. %, at least 5 wt. %, at least 10 wt. %, at least 20 wt. %, at least 30 wt. %, at least 40 wt. %, at least 50 wt. %, at least 60 wt. %, at least 70 wt. %, at least 80 wt. %, at least 90 wt. %, at least 95 wt. %, at least 99 wt. %, at least 99.9 wt. %, or up to 100 wt. % of the second solute from the feed liquid stream.

In some embodiments, the first liquid inlet of the first stage is fluidically connected to a source containing a first liquid phase and to a liquid outlet of at least one of the one or more intermediate separator stage(s). The first liquid inlet, in some embodiments, may be configured to receive the first liquid phase from the source containing the first liquid phase and at least a portion of a liquid from the liquid outlet of the at least one intermediate separator stage. FIG. 2A illustrates an example of one such set of embodiments. As shown in FIG. 2A, first liquid inlet 122 of first separator stage 120 is fluidically connected to source 114 containing the first liquid phase and fluidically connected to liquid outlet 146 of intermediate separator stage 140. In some instances, first liquid inlet 122 may be configured to receive first liquid phase 114a from source 114 containing the first liquid phase and at least a portion of liquid 146a from liquid outlet 146 of intermediate separator stage 140.

In FIG. 2A, the fluidic connectivity between first liquid inlet 122 and intermediate liquid outlet 146 may be either a direct fluidic connectivity or an indirect fluidic connectivity. For example, in embodiments in which no additional intermediate separator stages are present between intermediate separator stage 140 and first separator stage 120, first liquid inlet 122 is in direct fluidic connectivity with liquid outlet 146 of intermediate separator stage 140. In some such embodiments, first liquid inlet 122 may be configured to receive all of liquid 146a from liquid outlet 146 of intermediate separator stage 140. On the contrary, as shown in FIG. 2B, in embodiments in which one or more additional intermediate separator stages (e.g., first intermediate separator stage 130) are present between intermediate separator stage 140 and first separator stage 120, first liquid inlet 122 is in indirect fluidic connectivity with liquid outlet 146 of intermediate separator stage 140. As such, first liquid inlet 122 may be configured to receive only a portion of liquid 146a from liquid outlet 146 of intermediate separator stage 140 after liquid 146a has passed through the one or more additional intermediate separator stage(s). Depending on whether any additional intermediate separator stages are present and/or the number of additional intermediate separator stages between intermediate separator stage 140 and first separator stage 120, first liquid inlet 122 may be configured to receive at least 0.01 wt. % (e.g., at least 0.05 wt. %, at least 0.1 wt. %, at least 0.5 wt. %, at least 1 wt. %, at least 5 wt. %, at least 10 wt. %, at least 20 wt. %, at least 30 wt. %, at least 40 wt. %) and/or up to 50 wt. % (e.g., up to 60 wt. %, up to 70 wt. %, up to 80 wt. %, up to 90 wt. % up to 95 wt. %, or 100 wt. %) of liquid 146a from liquid outlet 146 of intermediate separator stage 140.

In some embodiments, a mixing region may be fluidically connected to the first liquid inlet of the first separator stage. The mixing region, in certain embodiments, may be a region disposed along the fluidic connectivity between the first liquid inlet of the first separator stage and a liquid outlet of an intermediate separator stage. FIG. 2A illustrates an example of one such set of embodiments. As shown in FIG. 2A, mixing region 128 may be fluidically connected to first liquid inlet 122 of first separator stage 120. In some cases, mixing region 128 may be disposed along the fluidic connectivity between first liquid inlet 122 of first separator stage 120 and liquid outlet 146 of intermediate separator stage 140.

In some embodiments, the mixing region fluidically connected to the first liquid inlet may be configured to combine and induce mixing between the first liquid phase from the source containing the first liquid phase and a liquid (e.g., a liquid comprising predominantly the second liquid phase) from a liquid outlet of the intermediate separator stage, thereby forming a mixed liquid stream comprising two liquid phases (e.g., the first liquid phase and the second liquid phase). The mixed liquid stream received by the first liquid inlet may comprise any appropriate composition and/or component described elsewhere herein, e.g., such as comprising at least a portion of the first solute from the feed liquid stream and at least a portion of the second solute from the feed liquid stream. The mixing region, by inducing mixing, may facilitate preferential association or partitioning of the first solute and the second solute into the liquid phases (e.g., the first liquid phase, the second liquid phase) within the mixed liquid stream. For example, in one set of embodiments, the mixing region may be configured to facilitate preferential association of the first solute with the first liquid phase and preferential association of the second solute with the second liquid phase.

FIG. 2A illustrates an example of one such set of embodiments. As shown in FIG. 2A, mixing region 128 may be configured to combine and induce mixing between first liquid phase 114a from source 114 and liquid 146a from liquid outlet 146 of intermediate separator stage 140, thereby forming mixed liquid stream 128a comprising two liquid phases, e.g., the first liquid phase and the second liquid phase. Mixed liquid stream 128a may comprise least a portion of the first solute from feed liquid stream 112a and at least a portion of the second solute from feed liquid stream 112a. By inducing mixing, mixing region 128 may facilitate preferential association or partitioning of the first solute and the second solute into the liquid phases (e.g., the first liquid phase, the second liquid phase) within mixed liquid stream 128a.

In some embodiments, the mixing region may be a part of any of a variety of mixing devices and/or systems. Non-limiting examples mixing devices and/or systems include channel junctions, vessels, static mixers, and stirrers.

As described in more detail below, the amount of the first solute and the second solute in each of the first liquid phase and the second liquid phase within the mixed liquid stream may depend on the partition coefficients of the solutes between the liquid phases, which is a measure of the ability of first solute and second solute to differentially partition between the first liquid phase and the second liquid phase. The first solute and/or the second solute may have any of a variety of appropriate partition coefficients, as described in more detail below.

The mixed liquid stream received by the first liquid inlet may comprise the first liquid phase and the second liquid phase in any of a variety of appropriate amounts. For example, in some embodiments, a mass ratio of the first liquid phase to the second liquid phase in the mixed liquid stream may be greater than or equal to 5:95, greater than or equal to 10:90, greater than or equal to 20:80, greater than or equal to 30:70, greater than or equal to 40:60, greater than or equal to 50:50, greater than or equal to 60:40, greater than or equal to 70:30, greater than or equal to 80:20, or greater than or equal to 90:10. In some embodiments, a mass ratio of the first liquid phase to the second liquid phase in the mixed liquid stream may be less than or equal to 95:5, less than or equal to 90:10, less than or equal to 80:20, less than or equal to 70:30, less than or equal to 60:40, less than or equal to 50:50, less than or equal to 40:60, less than or equal to 30:70, less than or equal to 20:80, or less than or equal to 10:90. Combinations of the above-referenced ranges are also possible (e.g., greater than or equal to 5:95 and less than or equal to 95:5). Other ranges are also possible. As would be understood by one of ordinary skill in the art, when a mass ratio of A:B is “greater than or equal to 10:90,” it means that, when the mass of component A that is present is divided by the mass of component B that is present, the resulting value is greater than or equal to 10/90 (i.e., greater than or equal to 0.111 repeating). Similarly, when a mass ratio of A:B is “less than or equal to 90:10,” it that means that, when the mass of component A that is present is divided by the mass of component B that is present, the resulting value is less than or equal to 90/10 (i.e., less than or equal to 9).

In some embodiments, the first separator stage comprises a liquid outlet configured to output a liquid having a mole fraction of the second solute relative to the sum of the first solute and the second solute that is larger than a mole fraction of the second solute relative to the sum of the first solute and the second solute in the feed liquid stream. FIGS. 2A-2B illustrate examples of one such set of embodiments. As shown in FIGS. 2A-2B, first separator stage 120 comprises liquid outlet 126 configured to output liquid 126a having a mole fraction of the second solute relative to the sum of the first solute and the second solute that is larger than a mole fraction of the second solute relative to the sum of the first solute and the second solute in feed liquid stream 112a. In some embodiments, the mole fraction of the second solute relative to the sum of the first solute and the second solute in the liquid (e.g., liquid 126a in FIGS. 2A-2B) enriched in the second solute relative to the feed liquid stream that is output by the liquid outlet of the first separator stage may be at least 1.01 times, at least 1.05 times, at least 1.1 times, at least 1.2 times, at least 1.5 times, at least 2 times, at least 3 times, at least 5 times, at least 10 times, at least 50 times, at least 100 times, at least 1000 times, or at least 10⁵ times (and/or up to 10⁶ times, up to 10⁷ times, up to 10⁸ times, or more) the mole fraction of the second solute relative to the total amount of the first solute and the second solute in the feed liquid stream (e.g., feed liquid stream 112a in FIGS. 2A-2B). Combination of the above-referenced ranges are possible (at least 1.01 times and up to 10⁸ times). Other ranges are also possible. As a non-limiting example, in some cases, the feed liquid stream (e.g., feed liquid stream 112a) may contain 50 mol of the first solute and 50 mol of the second solute, which means the mole fraction of the second solute relative to the total amount of the first solute and the second solute would be 0.5 (i.e., 50/100). The output liquid (e.g., liquid 126a) from the liquid outlet of the first separator stage may contain 5 mol of the first solute and 45 mol of the second solute, which means the mole fraction of the second solute relative to the total amount of the first solute and the second solute in the liquid (e.g., liquid 126a) would be 0.9 (i.e., 45/50). In this non-limiting example, the mole fraction of the second solute relative to the total amount of the first solute and the second solute in the output liquid is 1.8 times the mole fraction of the second solute relative to the total amount of the first solute and the second solute in the feed liquid stream (because 0.9 divided by 0.5 is 1.8). In this example, the output liquid would be said to be enriched in the second solute relative to the feed liquid stream because the mole fraction of the second solute relative to the total amount of the first solute and the second solute in the output liquid (e.g., liquid 126a) is higher than the mole fraction of the second solute relative to total amount of the first solute and the second solute in the feed liquid stream. As another non-limiting example, in some cases, the feed liquid stream may contain 50 mol of the first solute and 50 mol of the second solute, which means the mole fraction of the second solute relative to the total amount of the first solute and the second solute would be 0.5 (i.e., 50/100). The output liquid (e.g., liquid 126a) from the liquid outlet of the first separator stage may contain 45 mol of the first solute and 5 mol of the second solute, which means the mole fraction of the second solute relative to the total amount of the first solute and the second solute in the output liquid (e.g., liquid 126a) would be 0.1 (.e., 5/50). In this non-limiting example, the mole fraction of the second solute relative to the total amount of the first solute and the second solute in the output liquid (e.g., liquid 126a) is 0.2 times the mole fraction of the second solute relative to the total amount of the first solute and the second solute in the feed liquid stream (because 0.1 divided by 0.5 is 0.2). In this example, the output liquid (e.g., liquid 126a) would not be said to be enriched in the second solute relative to the feed liquid stream because the mole fraction of the second solute relative to the total amount of the first solute and the second solute in the output liquid (e.g., liquid 126a) is lower than the mole fraction of the second solute relative to the total amount of the first solute and the second solute in the feed liquid stream.

In accordance with certain embodiments, a liquid output from a liquid outlet of the first separator stage has a mole fraction of the second solute relative to the sum of the first solute and the second solute that is larger than a mole fraction of the second solute relative to the sum of the first solute and the second solute in the liquid (e.g., a mixed liquid stream) received by the first liquid inlet of the first separator stage. As shown in FIGS. 2A-2B, liquid 126a output from liquid outlet 126 of first separator stage 120 may have a mole fraction of the second solute relative to the sum of the first solute and the second solute that is larger than a mole fraction of the second solute relative to the sum of the first solute and the second solute in liquid 128a (e.g., a mixed liquid stream) received by first liquid inlet 122 of first separator stage 120. In some embodiments, the mole fraction of the second solute relative to the total amount of the first solute and the second solute in the liquid (e.g., liquid 126a in FIGS. 2A-2B) output from a liquid outlet of the first separator stage may be at least 1.01 times, at least 1.05 times, at least 1.1 times, at least 1.2 times, at least 1.5 times, at least 2 times, at least 3 times, at least 5 times, at least 10 times, at least 50 times, at least 100 times, or at least 500 times, (and/or up to 10³ times, or more) the mole fraction of the second solute relative to the total amount of the first solute and the second solute in the liquid (e.g., liquid 128a in FIGS. 2A-2B) received by the first stage inlet of the first separator stage. Combination of the above-referenced ranges are possible (at least 1.01 times and up to 10³ times). Other ranges are also possible.

In some embodiments, the second solute makes up a relatively high percentage of a total amount of the first solute and the second solute contained within the liquid (e.g., liquid 126a in FIGS. 2A-2B) that is output from the liquid outlet (e.g., liquid outlet 126) of the first separator stage (e.g., first separator stage 124). For example, in some embodiments, the second solute makes up at least 80 wt. % (e.g., at least 85 wt. %, at least 90 wt. %, at least 95 wt. %, at least 97 wt. %, at least 98 wt. %, at least 99 wt. %, at least 99.5 wt. %, at least 99.9 wt. %) and/or up to 99.99 wt. % (e.g., up to 100 wt. %) of the total amount of the first solute and the second solute contained within liquid 126a that is output by liquid outlet 126 of first separator stage 120. Combinations of the above-referenced ranges are possible (e.g., at least 80 wt. % and up to 100 wt. %). Other ranges are also possible. In one set of embodiments, the liquid output comprises a negligible amount, if any, of the first solute (e.g., such that second solute makes up 100 wt. % of total amount of solutes).

In some embodiments, the liquid having a mole fraction of the second solute relative to the sum of the first solute and the second solute that is larger than a mole fraction of the second solute relative to the sum of the first solute and the second solute in the feed liquid stream that is output from the liquid outlet of the first separator stage comprises predominantly the second liquid phase and a small amount, if any, of the first liquid phase. For example, as shown in FIGS. 2A-2B, liquid 126a having a mole fraction of the second solute relative to the sum of the first solute and the second solute that is larger than a mole fraction of the second solute relative to the sum of the first solute and the second solute in feed liquid stream 112a that is output from liquid outlet 126 of first separator stage 120 comprises predominantly the second liquid phase and a small amount, if any, of the first liquid phase.

In some embodiments, the first separator stage comprises a liquid outlet fluidically connected to an intermediate liquid inlet of at least one of the one or more intermediate separator stages. FIG. 2A illustrates an example of one such set of embodiments. As shown in FIG. 2A, first separator stage 120 comprises liquid outlet 124 fluidically connected to intermediate liquid inlet 142 of intermediate separator stage 140. As described in more detail below, depending on whether additional separator stage(s) are present between first separator stage 120 and intermediate separator stage 140, liquid outlet 124 of first separator stage 120 may be either directly or indirectly fluidically connected to intermediate separator stage 140.

In some embodiments, the liquid outlet of the first separator stage fluidically connected to the intermediate liquid inlet of the intermediate separator stage is configured to output a liquid having a mole fraction of the first solute relative to the sum of the first solute and the second solute that is larger than a mole fraction of the first solute relative to the sum of the first solute and the second solute in the liquid received by the first liquid inlet of the first separator stage. As shown in FIG. 2A, liquid outlet 124 of first separator stage 120, which is fluidically connected to intermediate liquid inlet 142 of intermediate separator stage 140, is configured to output liquid 124a having a mole fraction of the first solute relative to the sum of the first solute and the second solute that is larger than a mole fraction of the first solute relative to the sum of the first solute and the second solute in liquid 128a received by first liquid inlet 122 of first separator stage 120. In some embodiments, a mole fraction of the first solute relative to the total amount of the first solute and the second solute in liquid 124a may be at least 1.01 times, at least 1.05 times, at least 1.1 times, at least 1.2 times, at least 1.5 times, at least 2 times, at least 3 times, at least 5 times, at least 10 times, at least 50 times, at least 100 times, or at least 500 times (and/or up to 10³ times, or more) the mole fraction of the first solute relative to the total amount of the first solute and the second solute in liquid 128a received by first liquid inlet 122 of first separator stage 120. Combinations of the above-referenced ranges are possible (e.g., at least 1.01 times and up to 10³ times). Other ranges are also possible.

In some embodiments, the liquid having a mole fraction of the first solute relative to the sum of the first solute and the second solute that is larger than a mole fraction of the first solute relative to the sum of the first solute and the second solute in the liquid received by the first liquid inlet that is output by the liquid outlet of the first separator stage comprises predominantly the first liquid phase and a small amount, if any, of the second liquid phase. For example, as shown in FIGS. 2A-2B, liquid 124a having a mole fraction of the first solute relative to the sum of the first solute and the second solute that is larger than a mole fraction of the first solute relative to the sum of the first solute and the second solute in liquid 128a that is output by liquid outlet 124 of first separator stage 120 comprises predominantly the first liquid phase and a small amount, if any, of the second liquid phase.

In some embodiments, the liquid-liquid chromatographic separator system comprises one or more intermediate separator stages. In accordance with some embodiments, at least one of the one or more intermediate separator stages comprises an intermediate liquid inlet configured to receive liquid comprising at least a portion of the first solute from the feed liquid stream and at least a portion of the second solute from the feed liquid stream. FIGS. 2A-2B illustrate examples of one such set of embodiments. As shown in FIGS. 2A-2B, intermediate separator stage 140 comprises intermediate liquid inlet 142 configured to receive liquid 148a comprising at least a portion of the first solute from feed liquid stream 112a and at least a portion of the second solute from feed liquid stream 112a. In some embodiments, the liquid comprising at least a portion of the first solute and at least a portion of the second solute received by the intermediate liquid inlet is a mixed liquid stream comprising a first liquid phase and a second liquid phase distinct from the first liquid phase. The first liquid phase and the second liquid phase may comprise any of a variety of appropriate first liquid phases and second liquid phase phases described elsewhere herein. Details regarding the composition of the mixed liquid stream are provided in more detail below.

In some embodiments, the intermediate liquid inlet of at least one of the intermediate separator stage(s) is fluidically connected to (e.g., directly or indirectly fluidically connected to) the feed liquid inlet, a liquid outlet of the first separator stage, and a liquid outlet of the last separator stage. FIG. 2A illustrates an example of one such set of embodiments. As shown in FIG. 2A, intermediate liquid inlet 142 of intermediate separator stage 142 is directly fluidically connected to feed liquid inlet 112, fluidically connected to liquid outlet 124 of first separator stage 120 (directly or indirectly), and fluidically connected to liquid outlet 166 of last separator stage 160 (directly or indirectly).

In some embodiments, the intermediate liquid inlet may be configured to receive the feed liquid stream from the feed liquid inlet, at least a portion of the liquid from the liquid outlet of the first separator stage, and at least a portion of the liquid from the liquid outlet of the last separator stage. For example, as shown in FIG. 2A, intermediate liquid inlet 142 may be configured to receive feed liquid stream 112a from feed liquid inlet 112, at least a portion of liquid 124a from liquid outlet 124 of first separator stage 120, and at least a portion of liquid 166a from liquid outlet 166 of last separator stage 160. The relative amount of liquid 124a received from liquid outlet 124 of first separator stage 120 and/or liquid 166a from liquid outlet 166 of last separator stage 160 may depend on the fluidic connectivity between the associated separator stages (e.g., whether the fluidic connectivity is a direct fluidic connectivity or indirect fluidic connectivity).

For example, referring to FIG. 2A, the fluidic connectivity between intermediate liquid inlet 142 of intermediate separator stage 140 and liquid outlet 124 of first separator stage 120 may be either a direct fluidic connectivity or an indirect fluidic connectivity. For instance, in embodiments in which no additional intermediate separator stages are present between intermediate separator stage 140 and first separator stage 120, intermediate liquid inlet 142 is in direct fluidic connectivity with liquid outlet 124 of first separator stage 120. As such, in some such embodiments, intermediate liquid inlet 142 may be configured to receive all of liquid 124a from liquid outlet 124 of first separator stage 124. On the contrary, as shown in FIG. 2B, in embodiments in which one or more additional intermediate separator stages (e.g., additional separator stage 130) are present between intermediate separator stage 140 and first separator stage 120, intermediate liquid inlet 142 is in indirect fluidic connectivity with liquid outlet 124 of first separator stage 120. In some such embodiments, intermediate liquid inlet 142 may be configured to receive at least a portion of liquid 124a from liquid outlet 124 of first separator stage 120 after liquid 124a passes through the one or more additional intermediate separator stage(s) (e.g., additional intermediate separator stage 130). Depending on whether any additional intermediate separator stages are present and/or the number of additional intermediate separator stages between intermediate separator stage 140 and first separator stage 120, intermediate liquid inlet 142 may be configured to receive at least at 0.01 wt. % (e.g., at least 0.05 wt. %, at least 0.1 wt. %, at least 0.5 wt. %, at least 1 wt. %, at least 5 wt. %, at least 10 wt. %, at least 20 wt. %, at least 30 wt. %, at least 40 wt. %) and/or up to 50 wt. % (e.g., up to 60 wt. %, up to 70 wt. %, up to 80 wt. %, up to 90 wt. % up to 95 wt. %, or 100 wt. %) of liquid 124a from liquid outlet 124 of first separator stage 120. Combinations of the above-referenced ranges are possible (e.g., at least 0.01 wt. % and up to 100 wt. %). Other ranges are also possible.

Similarly, as shown in FIG. 2A, the fluidic connectivity between intermediate liquid inlet 142 of intermediate separator stage 140 and liquid outlet 166 of last separator stage 160 may be either a direct fluidic connectivity or an indirect fluidic connectivity. For instance, in embodiments in which no additional intermediate separator stages are present between intermediate separator stage 140 and last separator stage 160, intermediate liquid inlet 142 is in direct fluidic connectivity with liquid outlet 166 of last separator stage 160. As such, in some such embodiments, intermediate liquid inlet 142 may be configured to receive all of liquid 166a from liquid outlet 166 of last separator stage 160. On the contrary, as shown in FIG. 2B, in embodiments in which one or more additional intermediate separator stages (e.g., additional separator stage 150) are present between in intermediate separator stage 140 and last separator stage 160, intermediate liquid inlet 142 is in indirect fluidic connectivity with liquid outlet 166 of last separator stage 160. As such, intermediate liquid inlet 142 may be configured to receive at least a portion of liquid 166a from liquid outlet 166 of last separator stage 160 after liquid 166a passes through one or more additional intermediate separator stage(s) (e.g., additional intermediate separator stage 150). Depending on whether any additional intermediate separator stages are present and/or the number of additional intermediate separator stages between intermediate separator stage 140 and last separator stage 160, intermediate liquid inlet 142 may be configured to receive at least 0.01 wt. % (e.g., at least 0.05 wt. %, at least 0.1 wt. %, at least 0.5 wt. %, at least 1 wt. %, at least 5 wt. %, at least 10 wt. %, at least 20 wt. %, at least 30 wt. %, at least 40 wt. %) and/or up to 50 wt. % (e.g., up to 60 wt. %, up to 70 wt. %, up to 80 wt. %, up to 90 wt. % up to 95 wt. %, or 100 wt. %) of liquid 166a from liquid outlet 166 of last separator stage 160. Combinations of the above-referenced ranges are possible (e.g., at least 0.01 wt. % and up to 100 wt. %). Other ranges are also possible.

In some embodiments, the liquid-liquid chromatographic separator system comprises one or more mixing regions fluidically connected to (e.g., directly fluidically connected to) the intermediate liquid inlet of at least one of the one or more intermediate separator stages. FIG. 2A illustrates an example of one such set of embodiments. As shown in FIG. 2A, liquid-liquid chromatographic separator system 10a comprises mixing region 148 directly fluidically connected to intermediate liquid inlet 142 of intermediate separator stage 140.

In some embodiments, the mixing region fluidically connected to the intermediate liquid inlet may be configured to combine and induce mixing between at least a portion of a liquid (e.g., a liquid comprising predominately the first liquid phase) from a liquid outlet of the first separator stage and at least a portion of the liquid (e.g., a liquid comprising predominately the second liquid phase) from a liquid outlet of the last separator stage, thereby forming a mixed liquid stream comprising two liquid phases (e.g., the first liquid phase and the second liquid phase). The mixed stream, in certain embodiments, is further combined and mixed with the feed liquid stream at the feed liquid inlet. For example, as shown in FIG. 2A, mixing region 148 fluidically connected to intermediate liquid inlet 142 may be configured to combine and induce mixing between at least a portion of liquid 124a from liquid outlet 124 of first separator stage 120 and at least a portion of liquid 166a from liquid outlet 166 of last separator stage 160, thereby forming a mixed liquid stream comprising two liquid phases (e.g., the first liquid phase and the second liquid phase). The mixed stream may be further combined and mixed with feed liquid stream 112a at feed liquid inlet 112, thereby forming mixed liquid stream 148a. While two separate mixing regions are shown in FIG. 2A, it should be understood that, in other embodiments, all three streams can be mixed within the same mixing region. For example, in some embodiments, all three of streams 166a, 124a, and 112a can be mixed within the same mixing region.

While FIG. 2A illustrates a non-limiting embodiment of a feed liquid inlet in direct fluidic communication with a particular intermediate separator stage (e.g., intermediate separator stage 140), it should be understood that not all embodiments described herein are so limiting, and in other embodiments, the feed liquid inlet may be directly fluidically connected with another separator stage (e.g., additional separator stage(s), first separator stage, last separator stage). For example, referring to FIG. 2B, feed liquid inlet 112, instead of being directly fluidically connected to separator stage 140, may be directly fluidically connected to any other separator stage (e.g., separator stages 120, 130, 150, or 160).

Also, while FIG. 2A illustrates a non-limiting embodiment of a feed liquid inlet in direct fluidic communication with a particular mixing region (e.g., mixing region 148), it should be understood that not all embodiments described herein are so limiting, and in other embodiments, the feed liquid inlet may be directly fluidically connected with another mixing region. For example, referring to FIG. 2B, feed liquid inlet 112, instead of being directly fluidically connected to mixing region 148, may be directly fluidically connected to any other mixing region (e.g., mixing regions 128, 138, 158, or 168).

The mixing region associated with the intermediate liquid inlet may comprise and/or may be a part of any of a variety of mixing devices and/or systems, including any of those described elsewhere herein.

The mixed liquid stream (e.g., mixed liquid stream 148a in FIG. 2A) received by the intermediate liquid inlet may comprise any appropriate composition and/or component described elsewhere herein, e.g., such as comprising at least a portion of the first solute from the feed liquid stream and at least a portion of the second solute from the feed liquid stream. The mixing region, by inducing mixing, may facilitate preferential association or partitioning of the first solute and the second solute into the liquid phases (e.g., the first liquid phase, the second liquid phase) within the mixed liquid stream. For example, in one set of embodiments, the mixing region fluidically connected to the intermediate of the intermediate separator stage may be configured to facilitate preferential association of the first solute with the first liquid phase and preferential association of the second solute with the second liquid phase within the mixed liquid stream. In some embodiments, the first liquid phase within the mixed liquid stream received by the intermediate liquid inlet may have a mole fraction of the first solute relative to the sum of the first and second solute that is larger than a mole fraction of the first solute relative to the sum of the first and second solute in the second liquid phase, and the second liquid phase within the mixed liquid stream received by the intermediate liquid inlet may have a mole fraction of the second solute relative to the sum of the first and second solute that is larger than a mole fraction of the second solute relative to the sum of the first and second solute in the first liquid phase.

The mixed liquid stream received by the intermediate liquid inlet may comprise the first liquid phase and the second liquid phase in any appropriate proportions. For example, in some embodiments, a mass ratio of the first liquid phase to the second liquid phase in the mixed liquid stream received by the intermediate liquid inlet may be greater than or equal to 5:95, greater than or equal to 10:90, greater than or equal to 20:80, greater than or equal to 30:70, greater than or equal to 40:60, greater than or equal to 50:50, greater than or equal to 60:40, greater than or equal to 70:30, or greater than or equal to 80:20. In some embodiments, a mass ratio of the first liquid phase to the second liquid phase in the mixed liquid stream received by the intermediate liquid inlet may be less than or equal to 95:5, less than or equal to 90:10, less than or equal to 80:20, less than or equal to 70:30, less than or equal to 60:40, less than or equal to 50:50, less than or equal to 40:60, less than or equal to 30:70, or less than or equal to 20:80. Combinations of the above-referenced ranges are also possible (e.g., greater than or equal to 5:95 and less than or equal to 95:5). Other ranges are also possible.

In some embodiments, at least one of the one or more intermediate separator stages comprises an intermediate liquid outlet fluidically connected to a last liquid inlet of the last separator stage. FIG. 2A illustrates an example of one such set of embodiments. As shown in FIG. 2A, intermediate separator stage 140 comprises intermediate liquid outlet 144 fluidically connected to last liquid inlet 162 of last separator stage 160. As described in more detail below, depending on whether additional separator stage(s) are present between intermediate separator stage 140 and last separator stage 160, liquid outlet 144 of intermediate separator stage 140 may be either directly or indirectly fluidically connected to last liquid inlet 162 of last separator stage 160.

In some embodiments, the intermediate liquid outlet fluidically connected to the last liquid inlet of the last separator stage is configured to output a liquid having a mole fraction of the first solute relative to the sum of the first solute and the second solute that is larger than a mole fraction of the first solute relative to the sum of the first solute and the second solute in the liquid received by the intermediate liquid inlet. FIG. 2A illustrates an example of one such set of embodiments. As shown in FIG. 2A, liquid outlet 144 of intermediate separator stage 140, which is fluidically connected to last liquid inlet 162 of last separator stage 160, may be configured to output liquid 144a having a mole fraction of the first solute relative to the sum of the first solute and the second solute that is larger than a mole fraction of the first solute relative to the sum of the first solute and the second solute in liquid 148a received by intermediate liquid inlet 142. For example, in some embodiments, the mole fraction of the first solute relative to the total amount of the first solute and the second solute in liquid 144a may be may be at least 1.01 times, at least 1.05 times, at least 1.1 times, at least 1.2 times, at least 1.5 times, at least 2 times, at least 3 times, at least 5 times, at least 10 times, at least 50 times, at least 100 times, or at least 500 times (and/or up to 10³ times, or more) the mole fraction of the first solute relative to the total amount of the first solute and the second solute in liquid 148a received by intermediate liquid inlet 142 of intermediate separator stage 140. Combinations of the above-referenced ranges are possible (e.g., at least 1.01 times and up to 10³ times). Other ranges are also possible.

In some embodiments, the liquid having a mole fraction of the first solute relative to the sum of the first solute and the second solute that is larger than a mole fraction of the first solute relative to the sum of the first solute and the second solute in the liquid received by the intermediate liquid inlet that is output from the liquid outlet of the intermediate separator stage comprises predominantly the first liquid phase and a small amount, if any, of the second liquid phase. For example, as shown in FIGS. 2A-2B, liquid 144a having a mole fraction of the first solute relative to the sum of the first solute and the second solute that is larger than a mole fraction of the first solute relative to the sum of the first solute and the second solute in liquid 148a received by intermediate liquid inlet 142 comprises predominantly the first liquid phase as opposed to the second liquid phase.

In some embodiments, at least one of the one or more intermediate separator stages comprises an intermediate liquid outlet fluidically connected to the first liquid inlet of the first separator stage. FIG. 2A illustrates an example of one such set of embodiments. As shown in FIG. 2A, intermediate separator stage 140 comprises intermediate liquid outlet 146 fluidically connected to first liquid inlet 122 of first separator stage 120. As described elsewhere herein, depending on whether additional separator stage(s) are present between intermediate separator stage 140 and first separator stage 120, liquid outlet 146 of intermediate separator stage 140 may be either directly or indirectly fluidically connected to first liquid inlet 122 of first separator stage 120.

In some embodiments, the intermediate liquid outlet fluidically connected to the first liquid inlet of the first separator stage is configured to output a liquid having a mole fraction of the second solute relative to the sum of the first solute and the second solute that is larger than a mole fraction of the second solute relative to the sum of the first solute and the second solute in the liquid received by the intermediate liquid inlet. FIG. 2A illustrates an example of one such set of embodiments. As shown in FIG. 2A, liquid outlet 146 of intermediate separator stage 140, which is fluidically connected to first liquid inlet 122 of the first separator stage 120, is configured to output liquid 146a having a mole fraction of the second solute relative to the sum of the first solute and the second solute that is larger than a mole fraction of the second solute relative to the sum of the first solute and the second solute in liquid 148a received by intermediate liquid inlet 142. For example, in some embodiments, the mole fraction of the second solute relative to the total amount of the first solute and the second solute in liquid 146a may be at least 1.01 times, at least 1.05 times, at least 1.1 times, at least 1.2 times, at least 1.5 times, at least 2 times, at least 3 times, at least 5 times, at least 10 times, at least 50 times, at least 100 times, or at least 500 times (and/or up to 10³ times, or more) the mole fraction of the second solute relative to the total amount of the first solute and the second solute in liquid 148a received by intermediate liquid inlet 142 of intermediate separator stage 140. Combinations of the above-referenced ranges are possible (e.g., at least 1.01 times and up to 10³ times). Other ranges are also possible.

In some embodiments, the liquid having a mole fraction of the second solute relative to the sum of the first solute and the second solute that is larger than a mole fraction of the second solute relative to the sum of the first solute and the second solute in the liquid received by the intermediate liquid inlet that is output from the liquid outlet of the intermediate separator stage comprises predominantly the second liquid phase and a small amount, if any, of the first liquid phase. For example, as shown in FIGS. 2A-2B, liquid 146a having a mole fraction of the second solute relative to the sum of the first solute and the second solute that is larger than a mole fraction of the second solute relative to the sum of the first solute and the second solute in liquid 148a received by intermediate liquid inlet 142 comprises predominantly the second liquid phase and a small amount, if any, of the first liquid phase.

In some embodiments, the last separator stage comprises a last liquid inlet. The last liquid inlet, in certain embodiments, may be configured to receive liquid comprising at least a portion of the first solute from the feed liquid stream and at least a portion of the second solute from the feed liquid stream. As described in more detail below, the liquid comprising at least a portion of the first solute and at least a portion of the second solute received by the last liquid inlet is a mixed liquid stream comprising a first liquid phase and a second liquid phase distinct from the first liquid phase.

FIGS. 2A-2B illustrate examples of one such set of embodiments. As shown in FIGS. 2A-2B, last separator stage 160 comprises last liquid inlet 162 configured to receive liquid 168a comprising at least a portion of the first solute from feed liquid stream 112a and at least a portion of the second solute from feed liquid stream 112a. Liquid stream 168a may be a mixed liquid stream comprising a first liquid phase and a second liquid phase distinct from (e.g., immiscible with) the first liquid phase. The first liquid phase and the second liquid phase may comprise any of a variety of appropriate first liquid phases and second liquid phase phases described elsewhere herein.

In some embodiments, the last liquid inlet is fluidically connected to (e.g., directly fluidically connected to) a source containing the second liquid phase and fluidically connected to (e.g., directly fluidically connected to) a liquid outlet of at least one of the one of the intermediate separator stage(s). The last liquid inlet, in some embodiments, may be configured to receive the second liquid phase from the source containing the second liquid phase and at least a portion of a liquid from the liquid outlet of the at least one intermediate separator stage. FIG. 2A illustrates an example of one such set of embodiments. As shown in FIG. 2A, last liquid inlet 162 of last separator stage 160 is fluidically connected to source 116 containing the second liquid phase and fluidically connected to liquid outlet 144 of intermediate separator stage 140. In some instances, last liquid inlet 162 may be configured to receive second liquid phase 116a from source 116 containing the second liquid phase and at least a portion of liquid 144a from liquid outlet 144 of intermediate separator stage 140.

In FIG. 2A, the fluidic connectivity between last liquid inlet 162 and intermediate liquid outlet 144 may be either a direct fluidic connectivity or an indirect fluidic connectivity. For example, in embodiments in which no additional intermediate separator stages are present between intermediate separator stage 140 and last separator stage 160, last liquid inlet 162 is in direct fluidic connectivity with liquid outlet 144 of intermediate separator stage 140. As such, in some such embodiments, last liquid inlet 162 may be configured to receive all of liquid 144a from liquid outlet 144 of intermediate separator stage 140. On the contrary, as shown in FIG. 2B, in embodiments in which one or more additional intermediate separator stages (e.g., second intermediate separator stage 150) are present between intermediate separator stage 140 and last separator stage 160, last liquid inlet 162 is in indirect fluidic connectivity with liquid outlet 144 of intermediate separator stage 140. As such, last liquid inlet 162 may be configured to receive at least a portion of liquid 144a from liquid outlet 144 of intermediate separator stage 140 after liquid 144a passes through the one or more additional intermediate separator stage(s). Depending on whether any additional intermediate separator stages are present and/or the number of additional intermediate separator stages between intermediate separator stage 140 and last separator stage 160, last liquid inlet 162 may be configured to receive at least 0.01 wt. % (e.g., at least 0.05 wt. %, at least 0.1 wt. %, at least 0.5 wt. %, at least 1 wt. %, at least 5 wt. %, at least 10 wt. %, at least 20 wt. %, at least 30 wt. %, at least 40 wt. %) and/or up to 50 wt. % (e.g., up to 60 wt. %, up to 70 wt. %, up to 80 wt. %, up to 90 wt. % up to 95 wt. %, or 100 wt. %) of liquid 144a from liquid outlet 144 of intermediate separator stage 140.

In some embodiments, a mixing region may be fluidically connected to (e.g., directly fluidically connected to) the last liquid inlet of the last separator stage. The mixing region, in certain embodiments, may be a region disposed along the fluidic connectivity between the last liquid inlet of the last separator stage and a liquid outlet of the intermediate separator stage. FIG. 2A illustrates an example of one such set of embodiments. As shown in FIG. 2A, mixing region 168 is directly fluidically connected to last liquid inlet 162 of last separator stage 160. In some cases, mixing region 168 may be disposed along the fluidic connectivity between last liquid inlet 162 of last separator stage 160 and liquid outlet 144 of intermediate separator stage 140.

In some embodiments, the mixing region fluidically connected to the last liquid inlet may be configured to combine and induce mixing between the second liquid phase from the source containing the second liquid phase and a liquid (e.g., a liquid comprising predominantly the first liquid phase as opposed to the second liquid phase) from a liquid outlet of an intermediate separator stage, thereby forming a mixed liquid stream comprising two liquid phases (e.g., the first liquid phase and the second liquid phase). The mixed liquid stream received by the last liquid inlet may comprise any appropriate composition and/or component described elsewhere herein, e.g., such as comprising at least a portion of the first solute from the feed liquid stream and at least a portion of the second solute from the feed liquid stream. The mixing region, by inducing mixing, may facilitate movement or partitioning of the first solute and the second solute into the liquid phases (e.g., the first liquid phase, the second liquid phase) within the mixed liquid stream. For example, in one set of embodiments, the mixing region may be configured to facilitate preferential association of the first solute with the first liquid phase and preferential association of the second solute with the second liquid phase.

FIG. 2A illustrates an example of one such set of embodiments. As shown in FIG. 2A, mixing region 168 may be configured to combine and induce mixing between second liquid phase 116a from source 116 and liquid 144a from liquid outlet 144 of intermediate separator stage 140, thereby forming mixed liquid stream 168a comprising two liquid phases, e.g., the first liquid phase and the second liquid phase. Mixed liquid stream 168a may be configured to comprise least a portion of the first solute from feed liquid stream 112a and at least a portion of the second solute from feed liquid stream 112a. By inducing mixing, mixing region 168 may facilitate movement or partitioning of the first solute and the second solute into the liquid phases (e.g., the first liquid phase, the second liquid phase) within mixed liquid stream 168a.

The mixing region may comprise and/or may be a part of any of a variety of mixing devices and/or systems, including any of those described elsewhere herein.

The mixed liquid stream received by the last liquid inlet may comprise the first liquid phase and the second liquid phase in any appropriate proportions. For example, in some embodiments, a mass ratio of the first liquid phase to the second liquid phase in the mixed liquid stream received by the last liquid inlet may be greater than or equal to 5:95, greater than or equal to 10:90, greater than or equal to 20:80, greater than or equal to 30:70, greater than or equal to 40:60, greater than or equal to 50:50, greater than or equal to 60:40, greater than or equal to 70:30, or greater than or equal to 80:20. In some embodiments, a mass ratio of the first liquid phase to the second liquid phase in the mixed liquid stream may be less than or equal to 95:5, less than or equal to 90:10, less than or equal to 80:20, less than or equal to 70:30, less than or equal to 60:40, less than or equal to 50:50, less than or equal to 40:60, less than or equal to 30:70, or less than or equal to 20:80. Combinations of the above-referenced ranges are also possible (e.g., greater than or equal to 5:95 and less than or equal to 95:5). Other ranges are also possible.

The amount of the first solute and the second solute in each of the first liquid phase and the second liquid phase within the mixed liquid stream may depend on the partition coefficients of the solutes between the liquid phases, which is a measure of the ability of first solute and second solute to differentially partition between the first liquid phase and the second liquid phase. The first solute and/or the second solute may have any of a variety of appropriate partition coefficients, as described in more detail below.

In some embodiments, the last separator stage comprises a liquid outlet fluidically connected to an intermediate liquid inlet of at least one of the one or more intermediate separator stages. FIG. 2A illustrate an example of one such set of embodiments. As shown in FIG. 2A, last separator stage 160 comprises liquid outlet 166 fluidically connected to intermediate liquid inlet 142 of intermediate separator stage 140. As described elsewhere herein, depending on whether additional separator stage(s) are present between last separator stage 160 and intermediate separator stage 140, liquid outlet 166 of last separator stage 160 may be either directly or indirectly fluidically connected to intermediate separator stage 140.

In some embodiments, the liquid outlet of the last separator stage fluidically connected to the intermediate liquid inlet of the intermediate separator stage is configured to output a liquid having a mole fraction of the second solute relative to the sum of the first solute and the second solute that is larger than a mole fraction of the second solute relative to the sum of the first solute and the second solute in the liquid received by the last liquid inlet of the last separator stage. As shown in FIG. 2A, liquid outlet 166 of last separator stage 160, which is fluidically connected to intermediate liquid inlet 142, is configured to output liquid 166a having a mole fraction of the second solute relative to the sum of the first solute and the second solute that is larger than a mole fraction of the second solute relative to the sum of the first solute and the second solute in liquid 168a received by last liquid inlet 162 of last separator stage 160. In some embodiments, the mole fraction of the second solute relative to the total amount of the first solute and the second solute in liquid 166a may be at least 1.01 times, at least 1.05 times, at least 1.1 times, at least 1.2 times, at least 1.5 times, at least 2 times, at least 3 times, at least 5 times, at least 10 times, at least 50 times, at least 100 times, or at least 500 times (and/or up to 10³ times, or more) the mole fraction of the second solute relative to the total amount of the first solute and the second solute in liquid 168a received by last liquid inlet 162 of last separator stage 160. Combinations of the above-referenced ranges are possible (e.g., at least 1.01 times and up to 10³ times). Other ranges are also possible.

In some embodiments, the liquid having a mole fraction of the second solute relative to the sum of the first solute and the second solute that is larger than a mole fraction of the second solute relative to the sum of the first solute and the second solute in the liquid received by the last liquid inlet that is output by the liquid outlet of the last separator stage comprises predominantly the second liquid phase and a small amount, if any, of the first liquid phase. For example, as shown in FIGS. 2A-2B, liquid 166a having a mole fraction of the second solute relative to sum of the first solute and second solute that is larger than a mole fraction of the second solute relative to the sum of the first solute and the second solute in liquid 168a that is output by liquid outlet 166 may comprise predominantly the second liquid phase and a small amount, if any of the first liquid phase.

In some embodiments, the last separator stage comprises a liquid outlet configured to output a liquid having a mole fraction of the first solute relative to the sum of the first solute and the second solute that is larger than a mole fraction of the first solute relative to the sum of the first solute and the second solute in the feed liquid stream. FIGS. 2A-2B illustrate examples of one such set of embodiments. As shown in FIGS. 2A-2B, last separator stage 160 comprises liquid outlet 164 configured to output liquid 164a having a mole fraction of the first solute relative to the sum of the first solute and the second solute that is larger than a mole fraction of the first solute relative to the sum of the first solute and the second solute in feed liquid stream 112a. In some embodiments, the mole fraction of the first solute relative to the total amount of the first solute and the second solute in the liquid (e.g., liquid 164a in FIGS. 2A-2B) enriched in the first solute relative to the feed liquid stream that is output by the liquid outlet of the last separator stage may be at least 1.01 times, at least 1.05 times, at least 1.1 times, at least 1.2 times, at least 1.5 times, at least 2 times, at least 3 times, at least 5 times, at least 10 times, at least 50 times, at least 100 times, at least 1000 times, or at least 10⁵ times (and/or up to 10⁶ times, up to 10⁷ times, up to 10⁸ times, or more) the mole fraction of the first solute relative to the total amount of the first solute and the second solute in the feed liquid stream (e.g., feed liquid stream 112a in FIGS. 2A-2B). Combination of the above-referenced ranges are possible (e.g., at least 1.01 times and up to 10⁸ times). Other ranges are also possible.

In accordance with certain embodiments, a liquid output from a liquid outlet of the last separator stage has a mole fraction of the first solute relative to the sum of the first solute and the second solute that is larger than a mole fraction of the first solute relative to the sum of the first solute and the second solute in the liquid (e.g., a mixed liquid stream) received by the last liquid inlet of the last separator stage. As shown in FIGS. 2A-2B, liquid 164a output from liquid outlet 164 of last separator stage 160 may have a mole fraction of the first solute relative to the sum of the first solute and the second solute that is larger than a mole fraction of the first solute relative to the sum of the first solute and the second solute in liquid 168a (e.g., a mixed liquid stream) received by last liquid inlet 162 of last separator stage 160. In some embodiments, the mole fraction of the first solute relative to the total amount of the first solute and the second solute in the liquid (e.g., liquid 164a in FIGS. 2A-2B) output from the liquid outlet of the last stage separator may be at least 1.01 times, at least 1.05 times, at least 1.1 times, at least 1.2 times, at least 1.5 times, at least 2 times, at least 3 times, at least 5 times, at least 10 times, at least 50 times, at least 100 times, or at least 500 times (and/or up to 10³ times, or more) the mole fraction of the first solute relative to the total amount of the first solute and the second solute in the liquid (e.g., liquid 168a in FIGS. 2A-2B) received by the last stage inlet of the last separator stage. Combination of the above-referenced ranges are possible (e.g., at least 1.01 times and up to 10³ times). Other ranges are also possible.

In some embodiments, the first solute makes up a relatively high percentage of a total amount of the first solute and the second solute contained within the liquid (e.g., liquid 164a in FIGS. 2A-2B) that is output from the liquid outlet (e.g., liquid outlet 164) of the last separator stage (e.g., last separator stage 160). For example, in some embodiments, the first solute makes up at least 80 wt. % (e.g., at least 85 wt. %, at least 90 wt. %, at least 95 wt. %, at least 97 wt. %, at least 98 wt. %, at least 99 wt. %, at least 99.5 wt. %, at least 99.9 wt. %) and/or up to 99.99 wt. % (e.g., up to 100 wt. %) of the total amount of the first solute and the second solute contained within liquid 164a that is output by liquid outlet 164 of last separator stage 160. Combinations of the above-referenced ranges are possible (e.g., at least 80 wt. % and up to 100 wt. %). Other ranges are also possible. In one set of embodiments, the liquid output comprises a negligible amount, if any, of the second solute (e.g., such that first solute makes up 100 wt. % of total amount of solutes).

In some embodiments, the liquid having a mole fraction of the first solute relative to the sum of the first solute and the second solute that is larger than a mole fraction of the first solute relative to the sum of the first solute and the second solute in the feed liquid stream that is output from the liquid outlet of the last separator stage comprises predominantly the first liquid phase and a small amount, if any, of the second liquid phase. FIGS. 2A-2B illustrate examples of one such set of embodiments. As shown in FIGS. 2A-2B, liquid 164a having a mole fraction of the first solute relative to the sum of the first solute and the second solute that is larger than a mole fraction of the first solute relative to the sum of the first solute and the second solute in feed liquid stream 112a that is output from liquid outlet 164 of last separator stage 160 comprises predominantly the first liquid phase and little to none of the second liquid phase.

It should be understood that the additional intermediate separator stages (e.g., additional intermediate separator stages 130 and 150) illustrated in FIG. 2B may be similar to or the same as the intermediate separator stage (e.g., intermediate separator stage 140) illustrated in FIG. 2A. Similarly, the various input liquid streams (e.g., liquid 158a, liquid 138a) and/or output liquid streams (e.g., liquids 134a and 136a, liquids 154a and 156a) associated with the additional intermediate separator stages (e.g., additional intermediate separator stages 130 and 150) shown in FIG. 2B may comprise similar compositions as described elsewhere herein with respect to the corresponding input liquid stream (e.g., liquid 148a) and/or output liquid streams (e.g., liquids 144a and 146a) of the intermediate separator stage (e.g., intermediate separator stage 140) illustrated in FIG. 2A.

In some embodiments, a method for separating a first solute from a second solute in a feed liquid stream is described. The separation may be performed using the liquid-liquid chromatographic separator systems described herein. For example, the liquid-liquid chromatographic separator systems may comprise a plurality of separator stages (e.g., three or more separator stages) arranged in series with one another from a first separator stage to a last separator stage, with one or more intermediate stages positioned between the first separator stage and the last separator stage. Non-limiting examples of one such set of embodiments are described elsewhere herein and/or with respect to FIGS. 2A-2B.

Certain embodiments comprise transporting (e.g., continuously transporting) a feed liquid stream comprising a first solute and a second solute into a feed liquid inlet of a liquid-liquid chromatographic separator system. FIGS. 2A-2B illustrate examples of one such set of embodiments. For example, as shown in FIGS. 2A-2B, feed liquid stream 112a comprising a first solute and a second solute may be transported into feed liquid inlet 112 of liquid-liquid chromatographic separator systems 100a and 100b.

In some embodiments, the feed liquid stream feeds (e.g., either directly or indirectly) into at least one of one or more the intermediate separator stages before passing through the first separator stage or the last separator stage. FIGS. 2A-2B illustrate examples of one such set of embodiments. For example, as shown in FIG. 2A, feed liquid stream 112a may feed directly into intermediate separator stage 140 before passing through first separator stage 120 or last separator stage 160. In embodiments in which additional intermediate separator stages are present (e.g., as shown in FIG. 2B), feed liquid stream 112a may feed indirectly into additional intermediate separator stages 150 and 130 after first feeding into intermediate separator stage 140.

While FIGS. 2A-2B illustrate non-limiting embodiments of transporting the feed liquid stream directly into a particular intermediate separator stage (e.g., intermediate separator stage 140), it should be understood that not all embodiments described herein are so limiting, and in other embodiments, the feed liquid stream may be transported directly into a feed liquid inlet positioned adjacent any appropriate intermediate separator stage. For example, in some embodiments, the feed liquid stream may be transported into a feed liquid inlet positioned adjacent any additional intermediate separator stages (e.g., additional separator stage 130 and/or 150 as shown in FIG. 2B), e.g., such that the feed liquid stream feeds directly into the additional intermediate separator stages before passing through the first separator stage or the last separator stage.

In some embodiments, the method comprises transporting a first liquid phase from a source containing the first liquid phase into a first liquid inlet of the first separator stage. In accordance with some embodiments, the method comprises transporting a second liquid phase distinct from (e.g., immiscible with) the first liquid into a last liquid inlet of a last separator stage from a source containing the second liquid phase. FIGS. 2A-2B illustrate examples of one such set of embodiments. For example, as shown in FIGS. 2A-2B, first liquid phase 114a from source 114 containing the first liquid phase may be transported into first liquid inlet 122 of first separator stage 120, while second liquid phase 116a from source 116 containing the second liquid phase may be transported into last liquid inlet 162 of last separator stage 160.

Certain embodiments comprise transporting a liquid (e.g., a mixed liquid stream) comprising at least a portion of the first solute from the feed liquid stream and at least a portion of the second solute from the feed liquid stream into a first liquid inlet of a first separator stage. FIGS. 2A-2B illustrate examples of one such set of embodiments. For example, as shown in FIGS. 2A-2B, liquid 128a comprising at least a portion of the first solute from feed liquid stream 112a and at least a portion of the second solute from feed liquid stream 112a may be transported into first liquid inlet 122 of first separator stage 120. As mentioned elsewhere herein, the mixed liquid stream transported into the first liquid inlet may be a liquid comprising the first liquid phase and the second liquid phase distinct from (e.g., immiscible with) the first liquid phase.

In accordance with some embodiments, the mixed liquid stream transported into the first liquid inlet may be formed by combining, at a mixing region adjacent the first liquid inlet, the first liquid phase from the source containing the first liquid phase with a liquid from a liquid outlet of at least one of the one or more intermediate separator stages. In accordance with certain embodiments, the liquid stream from the liquid outlet of the at least one or the one or more intermediate separator stages may comprise predominantly the second liquid phase as opposed to the first liquid phase. FIG. 2A illustrates an example of one such set of embodiments. For example, as shown in FIG. 2A, mixed liquid stream 128a transported into first liquid inlet 122 may be formed by combining, at mixing region 128, first liquid phase 114a from source 114 containing the first liquid phase with liquid 146a from liquid outlet 146 of intermediate separator stage 140. In some cases, liquid 146a comprises predominantly the second liquid phase as opposed to the first liquid phase. As such, the resulting mixed liquid stream 128a may be a stream comprising the first liquid phase and the second liquid phase.

While FIG. 2A illustrates an non-limiting embodiment of combining the first liquid phase from the source containing the first liquid phase with a liquid from a liquid outlet of a particular intermediate separator stage (e.g., intermediate separator stage 140), it should be understood that not all embodiments described herein are so limiting, and in other embodiments, the first liquid phase from the source containing the first liquid phase may be combined with a liquid from a liquid outlet of any appropriate intermediate separator stage. For example, as shown in FIG. 2B, when one or more additional intermediate separator stages (e.g., intermediate separator stage 130) are present between intermediate separator stage 140 and first separator stage 120, first liquid phase 114a from source 114 containing the first liquid phase may be combined with liquid 136a from liquid outlet 136 of additional intermediate separator stage 130 to form mixed liquid stream 128a.

In some embodiments, as the liquid comprising at least a portion of the first solute and at least a portion of the second solute is transported into the first liquid inlet, the first separator stage produces a liquid having a mole fraction of the second solute relative to the sum of the first solute and the second solute that is larger than a mole fraction of the second solute relative to the sum of the first solute and the second solute in the feed liquid stream and a liquid having a mole fraction of the first solute relative to the sum of the first solute and the second solute that is larger than a mole fraction of the first solute relative to the sum of the first solute and the second solute in the liquid received by the first liquid inlet of the first separator stage. In accordance with certain embodiments, the liquid produced by the liquid outlet of the first separator stage may also have a mole fraction of the second solute relative to the sum of the first solute and the second solute that is larger than a mole fraction of the second solute relative to the sum of the first solute and the second solute in the liquid received by the first liquid inlet. For example, as shown in FIGS. 2A-2B, as liquid 128a comprising at least a portion of the first solute and at least a portion of the second solute is transported into first liquid inlet 122, first separator stage 120 produces liquid 126a having a mole fraction of the second solute relative to the sum of the first solute and the second solute that is larger than a mole fraction of the second solute relative to the sum of the first solute and the second solute in feed liquid stream 112a and liquid 124a having a mole fraction of the first solute relative to the sum of the first solute and the second solute that is larger than a mole fraction of the first solute relative to the sum of the first solute and the second solute in liquid 128a received by first liquid inlet 122 of first separator stage 120. Liquid 126a produced by liquid outlet 126 of first separator stage 120 may also have a mole fraction of the second solute relative to the sum of the first solute and the second solute that is larger than a mole fraction of the second solute relative to the sum of the first solute and the second solute in liquid 128a received by first liquid inlet 122. The liquid comprising at least a portion of the first solute and at least a portion of the second solute may comprise any of a variety of appropriate amounts of the first solute and the second solute described elsewhere herein and/or with respect to FIGS. 2A-2B.

Certain embodiments comprise transporting a liquid (e.g., a mixed liquid stream) comprising at least a portion of the first solute and at least a portion of the second solute into an intermediate liquid inlet of at least one of the one or more intermediate separator stages. FIG. 2A illustrates an example of one such set of embodiments. As shown in FIG. 2A, liquid 148a (e.g., a mixed liquid stream) comprising at least a portion of the first solute and at least a portion of the second solute may be transported into intermediate liquid inlet 142 of intermediate separator stage 140. As mentioned elsewhere herein, the mixed liquid stream transported into the intermediate liquid inlet can comprise the first liquid phase and the second liquid phase.

In accordance with some embodiments, the mixed liquid stream transported into the intermediate liquid inlet may be formed by combining, at a mixing region adjacent the intermediate liquid inlet, a liquid stream from a liquid outlet of a preceding separator stage and a liquid stream from a liquid outlet of a next separator stage. Depending on the number of intermediate separator stage(s) present and their relative placement in the liquid-liquid chromatographic separator system, the preceding separator stage may either be another intermediate separator stage or the first separator stage. Similarly, the next separator stage may either be another intermediate separator stage or the last separator stage. In some cases, the liquid stream from the outlet of the preceding separator stage is a liquid comprising predominantly the first liquid phase as opposed to the second liquid phase, while the liquid stream from the outlet of the next separator stage is a liquid comprising predominantly the second liquid phase as opposed to the first liquid phase. Accordingly, a combination of the two liquid streams at the mixing region may result in the formation of the mixed liquid stream comprising two distinct (e.g., immiscible) phases. In some embodiments, the mixed liquid stream may be further combined with a feed liquid stream before being transported into the intermediate liquid inlet.

FIG. 2A illustrates an example of one such set of embodiments for a liquid-liquid chromatographic separator system comprising three or more separator stages. For example, as shown in FIG. 2A, mixed liquid stream 148a (e.g., a mixed liquid stream) transported into intermediate liquid inlet 142 may be formed by combining, at mixing region 148 adjacent intermediate liquid inlet 142, liquid 124a from liquid outlet 124 of first separator stage 120 (e.g., the preceding separator stage) and liquid 166a from liquid outlet 166 of last separator stage 160 (e.g., the next separator stage). While liquid 124a comprises predominantly the first liquid phase as opposed to the second liquid phase, liquid 166a comprises predominantly the second liquid phase as opposed to the first liquid phase. Mixed liquid stream 148a may be further combined with feed liquid stream 112a prior being transported into intermediate stage inlet 142 of intermediate separator stage 140.

FIG. 2B illustrates an example of one such set of embodiments for a liquid-liquid chromatographic separator system comprising five or more separator stages. For example, as shown in FIG. 2B, mixed liquid stream 48a transported into intermediate liquid inlet 142 may be formed by combining, at mixing region 148 adjacent intermediate liquid inlet 142, liquid 134a from liquid outlet 134 of first additional intermediate separator stage 130 (e.g., the preceding separator stage) and liquid 156a from liquid outlet 156 of second additional intermediate separator stage 150 (e.g., the next separator stage). While liquid 134a comprises predominantly the first liquid phase as opposed to the second liquid phase, liquid 156a comprises predominantly the second liquid phase as opposed to the first liquid phase. Mixed liquid stream 148a may be further combined with feed liquid stream 112a prior being transported into intermediate stage inlet 142 of intermediate separator stage 140.

In some embodiments, as the liquid comprising at least a portion of the first solute and at least a portion of the second solute is transported into the intermediate liquid inlet of at least one of the one or more intermediate separator stages, the at least one intermediate separator stage produces a liquid having a mole fraction of the first solute relative to the sum of the first solute and the second solute that is larger than a mole fraction of the first solute relative to the sum of the first solute and the second solute in the liquid received by the intermediate liquid inlet, and a liquid having a mole fraction of the second solute relative to the sum of the first solute and the second solute that is larger than a mole fraction of the second solute relative to the sum of the first solute and the second solute in the liquid received by the intermediate liquid inlet. FIG. 2A illustrates an example of one such set of embodiments. As shown in FIG. 2A, as liquid 148a (e.g., a mixed liquid stream) comprising at least a portion of the first solute and at least a portion of the second solute is transported into intermediate liquid inlet 142 of intermediate separator stage 140, intermediate separator stage 140 produces liquid 144a having a mole fraction of the first solute relative to the sum of the first solute and the second solute that is larger than a mole fraction of the first solute relative to the sum of the first solute and the second solute in liquid 148a received by intermediate liquid inlet 142, and liquid 146a having a mole fraction of the second solute relative to the sum of the first solute and the second solute that is larger than a mole fraction of the second solute relative to the sum of the first solute and the second solute in liquid 148a received by intermediate liquid inlet 142.

It should be understood that the additional intermediate separator stages (e.g., additional intermediate separator stages 130 and 150) shown in FIG. 2B may function in a similar manner as the intermediate separator stage (e.g., intermediate separator stage 140) shown in FIG. 2A. For example, for each of the additional intermediate separator stages (e.g., separator stage 130 or 150), a mixed liquid stream (e.g., liquid 138a or 158a) comprising at least a portion of the first solute and at least a portion of the second solute may be transported into the intermediate liquid inlet (e.g., inlet 132 or 152) of the corresponding additional intermediate separator stage, thereby producing a liquid (e.g., liquid 134a or 154a) having a mole fraction of the first solute relative to the sum of the first solute and the second solute that is larger than a mole fraction of the first solute relative to the sum of the first solute and the second solute in the liquid (e.g., liquid 138a or 158a) received by the intermediate liquid inlet (e.g., inlet 132 or 152), and a liquid (e.g., liquid 136a or 156a) having a mole fraction of the second solute relative to the sum of the first solute and the second solute that is larger than a mole fraction of the second solute relative to the sum of the first solute and the second solute in the liquid (e.g., liquid 138a or 158a) received by the intermediate liquid inlet (e.g., inlet 132 or 152).

Certain embodiments comprise transporting at least a portion of the liquid produced by the intermediate separator stage (and having a mole fraction of the second solute relative to the sum of the first solute and the second solute that is larger than a mole fraction of the second solute relative to the sum of the first solute and the second solute in the liquid received by the intermediate liquid inlet) into a liquid inlet of a preceding separator stage. Depending on the number of intermediate separator stage(s) present and their relative placement in the liquid-liquid chromatographic separator system, the preceding separator stage may either be another intermediate separator stage or the first separator stage. FIGS. 2A-2B illustrate examples of one such set of embodiments. For example, as shown in FIG. 2A, liquid 146a that is produced by intermediate separator stage 140 may be transported into first liquid inlet 122 of first separator stage 120 (e.g., the preceding separator stage). For another example, as shown in FIG. 2B, when one or more additional intermediate separator stages (e.g., second additional intermediate stage 130) are present between intermediate separator stage 140 and first separator stage 120, liquid 146a that is produced by intermediate separator stage 140 may be instead transported to liquid inlet 132 of first additional intermediate separator stage 130, before being subsequently transported to first liquid inlet 122 of first separator stage 120.

Certain embodiments comprise transporting at least a portion of the liquid that is produced by at least one of the one or more intermediate separator stages (and having a mole fraction of the first solute relative to the sum of the first solute and the second solute that is larger than a mole fraction of the first solute relative to the sum of the first solute and the second solute in the liquid received by the intermediate liquid inlet) into a liquid inlet of the next separator stage. Depending on the number of intermediate separator stage(s) present and their relative placement in the liquid-liquid chromatographic separator system, the next separator stage may either be another intermediate separator stage or the last separator stage. FIGS. 2A-2B illustrate examples of one such set of embodiments. For example, as shown in FIG. 2A, liquid 144a that is produced by intermediate separator stage 140 may be transported into last liquid inlet 162 of last separator stage 160 (e.g., the next separator stage). For another example, as shown in FIG. 2B, when additional intermediate separator stages (e.g., second additional intermediate stage 150) are present between intermediate separator stage 140 and last separator stage 160, liquid 144a that is produced by intermediate separator stage 140 may be instead transported to liquid inlet 152 of second additional intermediate separator stage 150, before being transported to last liquid inlet 162 of last separator stage 160.

Certain embodiments comprise transporting a liquid (e.g., a mixed liquid stream) comprising at least a portion of the first solute and at least a portion of the second solute into a last liquid inlet of the last separator stage. For example, as shown in FIGS. 2A-2B, liquid 168a comprising at least a portion of the first solute and at least a portion of the second solute may be transported into last liquid inlet 162 of last separator stage 160. As mentioned elsewhere herein, the mixed liquid stream transported into the last liquid inlet may be a liquid comprising the first liquid phase and the second liquid phase distinct from (e.g., immiscible with) the first liquid phase.

In accordance with some embodiments, the mixed liquid stream transported into the last liquid inlet may be formed by combining, at a mixing region adjacent the last liquid inlet, the second liquid phase from the source containing the second liquid phase with a liquid from a liquid outlet of at least one of the one or more intermediate separator stages. In accordance with certain embodiments, the liquid stream from the liquid outlet of the at least one of the one or more intermediate separator stages may comprise predominantly the first liquid phase as opposed to the second liquid phase. FIG. 2A illustrates an example of one such set of embodiments. For example, as shown in FIG. 2A, mixed liquid stream 168a transported into last liquid inlet 162 may be formed by combining, at mixing region 168, second liquid phase 116a from source 116 containing the second liquid phase with liquid 144a from liquid outlet 144 of intermediate separator stage 140. In some cases, liquid 144a comprises predominantly the first liquid phase as opposed to the second liquid phase. As such, the resulting mixed liquid stream 168a may be a stream comprising the first liquid phase and the second liquid phase.

While FIG. 2A illustrates a non-limiting embodiment of combining, at a mixing region, the second liquid phase from the source containing the second liquid phase with a liquid from a liquid outlet of a particular intermediate separator stage (e.g., intermediate separator stage 140), it should be understood that not all embodiments described herein are so limiting, and in other embodiments, the second liquid phase from the source containing the second liquid phase may be combined with a liquid from a liquid outlet of any appropriate intermediate separator stage. For example, as shown in FIG. 2B, when one or more additional intermediate separator stages (e.g., intermediate separator stage 150) are present between intermediate separator stage 140 and last separator stage 160, second liquid phase 116a from source 116 containing the second liquid phase may be combined with liquid 154a from liquid outlet 154 of additional intermediate separator stage 150 to form mixed liquid stream 168a.

In some embodiments, as the liquid comprising at least a portion of the first solute and at least a portion of the second solute is transported into the last liquid inlet of the last separator stage, the last separator stage produces a liquid having a mole fraction of the first solute relative to the sum of the first solute and the second solute that is larger than a mole fraction of the first solute relative to the sum of the first solute and the second solute in the feed liquid stream, and a liquid having a mole fraction of the second solute relative to the sum of the first solute and the second solute that is larger than a mole fraction of the second solute relative to the sum of the first solute and the second solute in the liquid received by the last liquid inlet. In accordance with certain embodiments, the liquid produced by the liquid outlet of the last separator stage may also have a mole fraction of the first solute relative to the sum of the first solute and the second solute that is larger than a mole fraction of the first solute relative to the sum of the first solute and the second solute in the liquid received by the last liquid inlet. For example, as shown in FIGS. 2A-2B, as liquid 168a comprising at least a portion of the first solute and at least a portion of the second solute is transported into last liquid inlet 162 of last separator stage 160, last separator stage 162 produces liquid 164a having a mole fraction of the first solute relative to the sum of the first solute and the second solute that is larger than a mole fraction of the first solute relative to the sum of the first solute and the second solute in the feed liquid stream, and liquid 166a having a mole fraction of the second solute relative to the sum of the first solute and the second solute that is larger than a mole fraction of the second solute relative to the sum of the first solute and the second solute in liquid 168a received by last liquid inlet 162. Additionally, liquid 164a produced by liquid outlet 164 of last separator stage 160 may also have a mole fraction of the first solute relative to the sum of the first solute and the second solute that is larger than a mole fraction of the first solute relative to the sum of the first solute and the second solute in liquid 168a received by last liquid inlet 162.

As noted above, certain embodiments are directed to liquid-liquid chromatographic separator systems and associated methods. As used herein, a “liquid-liquid chromatographic separator” is one in which two liquid phases are used to separate two solutes, with at least one set of streams in the system becoming more and more enriched with one of the solutes as one moves from stage to stage. For example, in FIG. 2A, output streams 144a and 164a become more and more enriched in the first solute as one moves from stage 140 to stage 160 (i.e., from right to left in FIG. 2A). Similarly, in FIG. 2B, output streams 144a, 154a, and 164a become more and more enriched in the first solute as one moves from stage 140 to stage 150 to stage 160 (i.e., from right to left in FIG. 2B). In some such embodiments, at least one set of streams in the system becomes more and more enriched with the second solute as one moves from stage to stage in the opposite direction. For example, in FIG. 2A, output streams 146a and 126a become more and more enriched in the second solute as one moves from stage 140 to stage 120 (i.e., from left to right in FIG. 2A). Similarly, in FIG. 2B, output streams 146a, 136a, and 126a become more and more enriched in the second solute as one moves from stage 140 to stage 130 to stage 120 (i.e., from left to right in FIG. 2B).

In some embodiments, the liquid-liquid chromatographic separator system described herein is configured to be operated continuously. A system is said to be operating “continuously” when, for at least a period of time, the system takes in an input and outputs an output. For example, the system can be operated continuously when a feed liquid stream comprising the first solute and the second solute is transported into the system while, at the same time, a liquid stream having a mole fraction of the first solute relative to the sum of the first solute and the second solute that is larger than a mole fraction of the first solute relative to the sum of the first solute and the second solute in the feed liquid stream is output from the system. In some embodiments, for at least a period of time, a feed liquid stream comprising the first solute and the second solute is transported into the system while, at the same time, a liquid stream having a mole fraction of the first solute relative to the sum of the first solute and the second solute that is larger than a mole fraction of the first solute relative to the sum of the first solute and the second solute in the feed liquid stream is output from the system and a liquid stream having a mole fraction of the second solute relative to the sum of the first solute and the second solute that is larger than a mole fraction of the second solute relative to the sum of the first solute and the second solute in the feed liquid stream is output from the system. Advantages associated with a continuous operation may include, but are not limited to, high-throughput of purified liquids stream containing a target solute, reduced amount of extraction liquid, reduced number of extraction stages associated with the separation process, and reduced overall operational costs.

In some embodiments, the liquid-liquid chromatographic separator system is a counter-current liquid-liquid chromatographic separator system. In a counter-counter liquid-liquid chromatographic separator system, two liquid phases (e.g., a first liquid phase, a second liquid phase) flow from stage to stage in opposite directions (e.g., such that two liquids, e.g., one liquid comprising predominantly the first liquid phase and having a mole fraction of the first solute relative to the sum of the first solute and the second solute that is larger than a mole fraction of the first solute relative to the sum of the first solute and the second solute in the feed liquid stream and the other liquid comprising the second liquid phase and having a mole fraction of the second solute relative to the sum of the first solute and the second solute that is larger than a mole fraction of the second solute relative to the sum of the first solute and the second solute in the feed liquid stream, may be produced). Non-limiting examples of counter-current liquid-liquid chromatographic separator systems are illustrated in FIGS. 2A-2B. As shown in FIGS. 2A-2B, in counter-current liquid-liquid chromatographic separator systems 100a and 100b, first liquid phase 114a and second liquid phase 116a may enter into the systems from opposite sides of the separation system and flow, from stage to stage, in opposite directions. Two liquids, e.g., liquid 164a comprising predominantly the first liquid phase and having a mole fraction of the first solute relative to the sum of the first solute and the second solute that is larger than a mole fraction of the first solute relative to the sum of the first solute and the second solute in the feed liquid stream, and liquid 126a comprising predominantly the second liquid phase and having a mole fraction of the second solute relative to the sum of the first solute and the second solute that is larger than a mole fraction of the second solute relative to the sum of the first solute and the second solute in the feed liquid stream, may be produced.

The first solute and the second solute may have any of a variety of partition coefficients between the first liquid phase and the second liquid phase. The first solute may have a partition coefficient K₁, which is expressed as a ratio of the concentration of the first solute in the first liquid phase to the concentration of the first solute in the second liquid phase at equilibrium (e.g., K₁=C_{1 (1st liquid phase)}/C_{1, (2nd liquid phase)}). Similarly, the second solute may have a partition coefficient K₂, where K₂ is expressed as a ratio of the concentration of the second solute in the first liquid phase to the concentration of the second solute in the second liquid phase (e.g., K₂=C_{2 (1st liquid phase)}/C_{2 (2nd liquid phase)}).

In some embodiments, the first solute has a partition coefficient K₁ between the first liquid phase and the second liquid phase of greater than or equal to 0.1, greater than or equal to 0.25, greater than or equal to 0.5, greater than or equal to 0.75, greater than or equal to 1, greater than or equal to 1.05, greater than or equal to 1.1, greater than or equal to 1.15, greater than or equal to 1.2, greater than or equal to 1.25, greater than or equal to 1.3, greater than or equal to 1.4, greater than or equal to 1.6, greater than or equal to 1.8, greater than or equal to 2, greater than or equal to 3, greater than or equal to 5, or greater than or equal to 10. In some embodiments, the first solute has a partition coefficient K₁ between the first liquid phase and the second liquid phase of up to 20, up to 40, up to 60, up to 80, up to 100, up to 200, up to 500, up to 1000, or greater. Combinations of the above-referenced ranges are possible (e.g., greater than or equal to 0.1 and up to 1000, greater than or equal to 1 and up to 100). Other ranges are also possible.

In some embodiments, the second solute has a partition coefficient K₂ between the first liquid phase and the second liquid phase of less than or equal to 10, less than or equal to 9, less than or equal to 8, less than or equal to 6, less than or equal to 4, less than or equal to 3, less than or equal to 2, less than or equal to 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, or less than or equal to 0.2 (and/or down to 0.01, down to 0.001, or less). Combinations of the above-referenced ranges are possible (e.g., less than or equal to 10 and down to 0.001, less than 1 and down to 0.001). Other ranges are also possible.

In some embodiments, it may be particularly advantageous to select a first liquid phase and a second liquid that gives rise to a partition coefficient K₁ for the first solute of greater than 1, and a partition coefficient K₂ for the second solute of less than 1. Such a combination of partition coefficients may result in a higher separation efficiency of the first solute and the second solute and may be associated with certain operational advantages (e.g., need for less solvent, lower number of extraction stages, etc.).

In some embodiments, the association of the chemical species (e.g., the first solute and the second solute) with their respective liquid phases (e.g., the first liquid phase, the second liquid phase) in the heterogeneous liquid mixture may correlate with the ability of the chemical species to selectively partition into the different liquid phases and the volumetric ratio between the different liquid phases. For example, in a biphasic heterogeneous liquid mixture comprising a first liquid phase and a second liquid phase, the association of a chemical species with the liquid phases may correlate with an extraction factor Y. For example, for chemical species i, the extraction factor Yᵢ may be expressed as: Yᵢ=Kᵢ·(V_{1st liquid phase}/V_{2nd liquid phase)}, which is the product of the partition coefficient Kᵢ for species i and a ratio of a volume factor of the first liquid phase (V_{1st liquid phase}) and a volume factor of the second liquid phase (V_{2nd liquid phase}). In cases where the separation process is a batch separation process, the volume factor of each phase is the volume of that phase that is present (i.e., in a batch separation process, V_{1st liquid phase} corresponds to the volume of the first liquid phase that is present, and V_{2nd liquid phase} corresponds to the volume of the second liquid phase that is present). In cases where the separation process is one in which the first and second phases are flowed (e.g., in a continuous separation process), the volume factor of each phase is the volumetric flow rate of that phase (i.e., in a separation process in which the phases are flowing, V_{1st liquid phase} corresponds to the volumetric flow rate of the first liquid phase, and V_{2nd liquid phase} corresponds to the volumetric flow rate of the second liquid phase). As noted elsewhere herein, for chemical species i, the partition coefficient Kᵢ may be expressed as: Kᵢ=C_{i (1st liquid phase)}/C_{i (2nd liquid phase)}, which is a ratio of the concentration of chemical species i in the first liquid phase (C_{i, 1st liquid phase}) to the concentration of chemical species i in the second liquid phase (C_{i, 2st liquid phase}).

In the context of the present disclosure, chemical species i may refer to the solute within the liquid phases. For example, in embodiments in which the mixture comprises a first solute and a second solute, the first solute may have an extraction factor Y₁, which, as described above, is expressed as a product of the partition coefficient Kᵢ of the first solute and the volume factor ratio (V_{1st liquid phase}/V_{2nd liquid phase}) between the first liquid phase and the second liquid phase, where K₁ is expressed as a ratio of the concentration of the first solute in the first liquid phase to the concentration of first solute in the second liquid phase (e.g., K₁=C_{1 (1st liquid phase)}/C_{1 (2nd liquid phase)}). Similarly, in embodiments in which the mixture comprises a first solute and a second solute, the second solute may have an extraction factor Y₂, which, as described above, is expressed as a product of the partition coefficient K₂ of the second solute and the volume factor ratio (V_{1st liquid phase}/V_{2nd liquid phase}) between the first liquid phase and the second liquid phase, where K₂ is expressed as a ratio of the concentration of the second solute in the first liquid phase to the concentration of second solute in the second liquid phase (e.g., K₂=C_{2 (1st liquid phase)}/C_{2 (2nd liquid phase)}).

In some embodiments, it may be advantageous to select a heterogeneous liquid mixture having a particular combination of extraction factors (e.g., Y₁, Y₂), e.g., such as an extraction factor Y₁ of the first solute of greater than 1 and an extraction factor Y₂ of the second solute of less than 1, or vice versa. Without wishing to be bound by any particular theory, it is hypothesized that such a particular combination of extraction factors may lead to efficient separation of the first solute from the second solute. For example, in some embodiments, the first solute may have an extraction factor Y₁ of greater than 1, greater than or equal to 1.05, greater than or equal to 1.1, greater than or equal to 1.15, greater than or equal to 1.2, greater than or equal to 1.25, greater than or equal to 1.3, greater than or equal to 1.4, greater than or equal to 1.6, greater than or equal to 1.8, greater than or equal to 2, or greater (and/or, in some embodiments, up to 2.5, up to 3, up to 4, up to 5, up to 6, up to 8, or up to 10, or more). Combinations of the above-referenced ranges are possible (e.g., greater than 1 and up to 10). Other ranges are also possible. Additionally, in some embodiments, the second solute may have an extraction factor Y₂ of less than 1, less than or equal to 0.99, less than or equal to 0.97, less than or equal to 0.95, less than or equal to 0.9, less than or equal to 0.85, less than or equal to 0.8, less than or equal to 0.7, less than or equal to 0.6, less than or equal to 0.4, less than or equal to 0.2, less than or equal to 0.1, or less (and/or down to 0.01, down to 0.001, or less). Combinations of the above-referenced ranges are possible (e.g., less than 1 and down to 0.001). Other ranges are also possible. In embodiments in which a multi-stage liquid-liquid extraction system is employed for separating the first solute from the second solute, the first stage, the last stage, and/or the one or more intermediate stages (and, in some embodiments, all of the first sage, the last stage, and the one or more intermediate stages) may have an extraction factor Y₁ for the first solute within any of the ranges outlined above. In embodiments in which a multi-stage liquid-liquid extraction system is employed for separating the first solute from the second solute, the first stage, the last stage, and/or the one or more intermediate stages (and, in some embodiments, all of the first sage, the last stage, and the one or more intermediate stages) may have an extraction factor Y₂ for the second solute within any of the ranges outlined above.

In some embodiments, the first liquid phase and the second liquid phase may comprise any of a variety of immiscible liquids. In one set of embodiments, the first liquid phase may be a polar liquid (e.g., a water miscible liquid), while the second liquid phase may be a non-polar liquid (e.g., a water insoluble organic phase). In some embodiments, the first liquid phase and the second liquid phase have mutual solubilities falling within any of the ranges outlined above.

In some embodiments, the liquid-liquid chromatographic separator system further comprises a temperature control system configured to control the temperature of the various liquid streams in the system. In one set of embodiments, the temperature control system may be advantageously coupled to one or more of the mixing regions and configured to the control the temperature of the liquid streams associated with the mixing region(s). FIG. 2A illustrates an example of one such set of embodiments. As shown in FIG. 2A, a temperature control system (not shown) may be advantageously coupled to one or more of the mixing region(s) (e.g., mixing regions 128, 148, 168, etc.) and configured to the control the temperature of the liquid streams (e.g., liquid 114a, 145a, 128a, 124a, 166a, 148a, 144a, 116a, 168a) associated with the mixing region(s).

As described elsewhere herein, the mixing region(s), by inducing mixing, may facilitate preferential association or partitioning of the first solute and the second solute into the liquid phases (e.g., the first liquid phase, the second liquid phase) within the mixed liquid stream(s). A temperature control system, by altering the temperature of the liquid streams, may be employed to alter the relative solubility of the first solute and second solute in each liquid phase, the mutual solubility between the liquid phases, and the partition coefficients of each solute. The use of a temperature control system, in accordance with certain embodiments, may allow for establishing desirable partitioning of the first solute and the second solute between the first liquid phase and the second liquid phase within the mixed liquid stream, and thereby enhancing the overall separation efficiency of the first solute and the second solute in the liquid-liquid chromatographic separator system.

As noted above, porous medium-based fluidic separators (membrane-based separators) may be employed in the separator stages described herein. Any of a variety of types of fluidic separators may be used as a separator stage, in accordance with certain of the embodiments described herein. In some embodiments, all of the fluidic separators within the separator stages may be of the same type (or may be essentially identical). In other embodiments, one or more of the separator stages in the system may be different from one or more other separator stages in the system.

As one example, a separator stage comprising a porous medium may be used (e.g., as the first separator stage, the last separator stage, and/or the intermediate separator stage(s), in certain embodiments. In some cases, the fluidic separator achieves separation through the use of interfacial tension within the pores of the porous medium. In some such cases, the pressure and/or volumetric flow rate of the incoming mixture must be sufficiently high to facilitate selective transport of the desired fluid through the pores of the porous medium while restricting transportation of the undesired fluid through the porous medium. Examples of such fluidic separators are described, for example, in International Patent Publication No. WO 2004/087283, published on Oct. 14, 2004, filed as International Patent Application No. PCT/US2004/009451 on Mar. 25, 2004, and entitled “Fluid Separation”; International Patent Publication No. WO 2007/006033, published on Jan. 11, 20017, filed as International Patent Application No. PCT/US2006/026464 on Jul. 5, 2006, and entitled “Microfluidic Separators for Multiphase Fluid-Flow Based on Membranes”; International Patent Publication No. WO 2014/026098, published on Feb. 13, 2014, filed as International Patent Application No. PCT/US2013/054312 on Aug. 9, 2013, and entitled “Pressure Control in Fluidic Systems”; and U.S. Pat. No. 10,987,671, issued on Apr. 27, 2021, and entitled “Reservoir-Based Management of Volumetric Flow Rate in Fluidic Systems,” each of which is incorporated herein by reference in its entirety for all purposes.

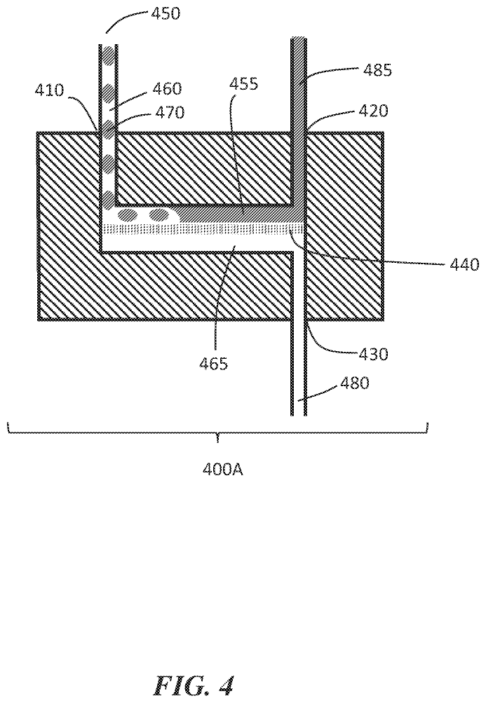

In certain embodiments, one or more of the separator stages comprises a porous medium-based fluidic separator. In certain instances, the porous medium separates the first outlet and the second outlet of one or more of the separator stages. One such exemplary separator (the type of which could be used as any of the separator stages described herein) is shown schematically in FIG. 4. In FIG. 4, separator stage 400A comprises porous medium 440 separating first outlet 420 of separator stage 400A and second outlet 430 of separator stage 400A. Non-limiting examples of porous media include porous membranes and porous discs (e.g., etched discs). In some embodiments, the porous medium of the separator comprises or is a porous membrane.

The solid portion of the porous medium can be made of any of a variety of materials including, but not limited to, metals, semiconductors, ceramics, polymers, and combinations thereof. In some embodiments, the solid portion of the porous medium comprises polyvinylidene fluoride (PVDF), polytetrafluoroethylene (PTFE), cellulose acetate, polypropylene, polyethylene, polysulfane, polyether sulfone, and/or polyvinyl chloride. In one set of embodiments, the porous medium comprises partially or fully fluorinated polymers, e.g., perfluorinated polymer(s).

According to certain embodiments, the fluidic combination transported into the separator stage comprising the porous medium comprises a first fluid and a second fluid. For example, in FIG. 4, fluidic combination 450, transported into inlet 410 of fluidic separator 400A, comprises first fluid 460 and fluid 470. The fluidic combination may be any of variety of mixed liquid streams described elsewhere herein and/or with respect to FIGS. 2A-2B, e.g., such as mixed liquid stream 128a associated with first stage separator 120, mixed liquid stream 148a associated with intermediate stage separator 140, mixed liquid stream 168a associated with last stage separator 160, mixed liquid stream 138a associated with first additional intermediate stage separator 130, mixed liquid stream 158a associated with second additional intermediate stage separator 160.

The first and second fluids can form separate phases, in some embodiments. An example of such is shown in FIG. 4, in which fluid 470 is shown as an immiscible slug within first fluid 460. In some embodiments, the first fluid is a first liquid phase and the second fluid is a second liquid phase that is immiscible in the first liquid phase. In certain cases, the fluidic combination comprises an emulsion. The first liquid phase and the second liquid phase may include any of a variety of first liquid phase and second liquid phases described elsewhere herein and/or with respect to FIGS. 2A-2B.

As noted above, in certain embodiments, the porous medium is pre-wetted with one liquid (e.g., a first liquid phase or a second liquid phase) from the fluidic combination (e.g., the mixed liquid phase). In some such embodiments, the liquid type that has been used to pre-wet the porous medium is selectively passed through the pre-wetted porous medium. As would be understood by those of ordinary skill in the art, “selective” transport of a first component through a porous medium (the “selectively transported component”) relative to another component (the “selectively retained component”) means that a higher percentage of the selectively transported component is transported through the porous medium, resulting in the formation of a fluid on the permeate side of the porous medium that contains a larger amount of the selectively transported component relative to the fluidic combination being transported into the separator, and a fluid on the retentate side of the porous medium that contains a larger amount of the selectively retained component relative to the fluidic combination being transported into the separator. For example, in FIG. 4, porous medium 440 has been pre-wetted with the solvent of first fluid 460, such that that solvent of the first fluid (and possibly, in some embodiments, some or all solutes dissolved therein) is selectively transported through the porous medium (e.g., with application of a hydraulic pressure to the retentate side of the porous medium) while fluid 470 is selectively retained by the porous medium. The selective transport of first fluid 460 through porous medium 440 results in the formation of fluid 455 on the retentate side of porous medium 440 that is has a larger amount of fluid 470 (the selectively retained component) relative to fluidic combination 450, and the formation of fluid 465 on the permeate side of porous medium 440 that has a larger amount of first fluid 460 (the selectively transported component) relative to fluidic combination 450.

In some instances, the pores within the porous medium within a separator are sized such that, when the porous medium is pre-wetted with one of the fluids within the incoming mixture, and the pressure of the incoming stream is sufficiently high, the pre-wetted fluid type is selectively transported through the porous medium while the other fluid(s) within the incoming mixture are selectively retained by the porous medium. Specific pore properties may be selected, in certain cases, to enhance the selectivity of the porous medium for a particular fluid.

In some embodiments, the pressure drop within one or more of the separator stages is relatively low. For examples, at least one (e.g. at least 2, at least 3, at least 4, at least 5, at least 7, at least 8, at least 10, at least 15, at least 20, at least 50, or all) of the separator stages may have a pressure drop of less than 0.5 bars, less than 0.3 bars, less than 0.1 bars, or less, and/or down to 0.05 bars, down to 0.01 bars, or less. Combinations of the above-referenced ranges are possible (e.g., less than 0.5 bars and down to 0.01 bars). Other ranges are also possible.

In some embodiments, the separation of chemical species is (substantially) independent of density of the chemical species and/or liquid phases. This may advantageously allow the use of a number of solvent systems for separation of chemical species, independent of their densities. In some embodiments, the separation of chemical separation is (substantially) independent of pH of the liquid phase and/or the environment within the separator system.

In some embodiments, the system described herein may be operated continuously or substantially continuously. The system and method described herein may allow for high throughput separation and purification of a wide variety of chemical species, at various scales (e.g., laboratory scale, pilot plant scale), according to some embodiments. For example, at a laboratory scale, chemical species within a sample containing two more chemical species may be separated from each other at rate of at least 1 mg/h, at least 10 mg/h, at least 0.1 g/h, at least 0.5 g/h, at least 1 g/h, at least 5 g/h, at least 10 g/h, at least 20 g/h, or more, and/or up to 30 g/h, up to 40 g/h, up to 50 g/h, or more. Combinations of the above-referenced ranges are possible (e.g., at least 1 mg/h and up to 50 g/h). Other ranges are also possible.

In some embodiments, at a pilot plant scale, chemical species within a sample containing two more chemical species may be separated from each other at rate of at least 1 mg/h, at least 10 mg/h, at least 0.1 g/h, at least 1 g/h, at least 10 g/h, at least 100 g/h, at least 1 kg/h, at least 10 kg/h, at least 50 kg/h, or more, and/or up to 100 kg/h, up to 200 kg/h, or more. Combinations of the above-referenced ranges are possible (e.g., at least 1 mg/h and up to 200 kg/h). Other ranges are also possible.

Any of a variety of chemical species may be separated from each other using the system and/or method described herein. In one set of embodiments, the chemical species include aldehydes. In one set of embodiments, the chemical species include chiral molecules, e.g., racemic drug molecules such as racemic propranolol. In some embodiments, the chemical species comprises natural products, e.g., components from depolymerized lignin.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

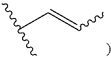

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and lor 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The following applications are incorporated herein by reference in their entireties for all purposes: U.S. Provisional Patent Application No. 63/352,587, filed Jun. 15, 2022, and entitled “Systems and Methods for Separation of Chemical Species, such as Cannabinoids, using Multiple Liquid Phases,” U.S. patent application Ser. No. 17/840,884, filed Jun. 15, 2022, and entitled “Continuous Liquid-Liquid Chromatographic Separation of Chemical Species Using Multiple Liquid Phases and Related Systems and Articles,” U.S. patent application Ser. No. 18/093,910, filed Jan. 6, 2023, and entitled “Separation of Chemical Species Using Multiple Liquid Phases and Related Systems,” and U.S. patent application Ser. No. 17/840,914, filed Jun. 15, 2022, and entitled “Separation of Chemical Species Using Multiple Liquid Phases and Related Systems.”

The following examples are intended to illustrate certain embodiments of the present invention, but do not exemplify the full scope of the invention.

### Examples 1-9

These examples describe the separation of delta-9-tetrahydrocannabinol from delta-8-tetrahydrocannabinol using various types of liquid mixtures, in accordance with certain embodiments.

A cannabinoid sample rich in overall tetrahydrocannabinol (THC) content (e.g., about 90% THC) with a low cannabidiol (CBD) content was exposed to various liquid mixtures in Examples 1-9. The cannabinoid sample contained both delta-9-tetrahydrocannabinol with delta-8-tetrahydrocannabinol. The liquid mixtures included an organic phase as the first liquid phase and a water soluble phase as the second liquid phase. The organic phase and the water soluble phase were in 1:1 volume ratio for all analyses. Various hydrocarbons were evaluated as the organic phase. These hydrocarbons included hexane, heptane, cyclohexane, pentane, 1-hexene, toluene, benzene, 1-octadecene, and dodecane. Various amide based solvents were evaluated as the water soluble phase. These amide based solvents included formamide (F), dimethyl formamide (DMF), methyl formamide (MF), dibutyl formamide (DBF).

Example 1. A mixture containing formamide (F) and dimethyl formamide (DMF) at a volume ratio of 9:16 (36% F and 64% DMF) was used as the water soluble phase. Hexane was used as the organic phase.

Example 2. A mixture containing formamide (F) and dimethyl formamide (DMF) at a volume ratio of 9:16 (36% F and 64% DMF) was used as the water soluble phase. Heptane was used as the organic phase.

Example 3. A mixture containing formamide (F) and dimethyl formamide (DMF) at a volume ratio of 9:16 (36% F and 64% DMF) was used as the water soluble phase. Cyclohexane was used as the organic phase.

Example 4. A mixture containing formamide (F) and dimethyl formamide (DMF) at a volume ratio of 9:16 (36% F and 64% DMF) was used as the water soluble phase. Pentane was used as the organic phase.

Example 5. A mixture containing formamide (F) and dimethyl formamide (DMF) at a volume ratio of 9:16 (36% F and 64% DMF) was used as the water soluble phase. 1-Hexene was used as the organic phase.

Example 6. A mixture containing formamide (F) and dimethyl formamide (DMF) at a volume ratio of 1:1 (50% F and 50% DMF) was used as the water soluble phase. Dodecane was used as the organic phase.

Example 7. A mixture of formamide (F) and methyl formamide (MF) at a volume ratio of 1:1 (50% F and 50% MF) was used as the water soluble phase. Hexane was used as the organic phase.

Example 8. A mixture of formamide (F) and methyl formamide (MF) at a volume ratio of 2:3 (40% F and 60% MF) was used as the water soluble phase. Hexane was used as the organic phase.

Example 9. A mixture of formamide (F) and dimethyl formamide (DMF) and dibutyl formamide (DBF) at a volume ratio of 1:3:1 (20% F and 60% DMF and 20% DBF) was used as the water soluble phase. Hexane was used as the organic phase. Table 1 shows the partition coefficients, K_{d9,THC} for delta-9-tetrahydrocannabinol and K_{d8, THC} for delta-8-tetrahydrocannabinol, in each liquid mixture. The partition coefficients for each component (e.g., delta-9-tetrahydrocannabinol, delta-8-tetrahydrocannabinol) in the liquid mixture were determined by calculating a ratio of the concentration of the component in the water soluble phase over concentration of the component in the organic phase.

TABLE 1
Partition coefficients for each liquid mixture.

| | Water soluble | | | | K_{d9},_{THC}/ |
|---|---|---|---|---|---|
| | phase | Organic Phase | K_{d9}, _{THC} | K_{d8}, _{THC} | K_{d8},_{THC} |
| Example 1 | F:DMF (9:16) | Hexane | 1.27 | 0.86 | 1.47 |
| Example 2 | F:DMF (9:16) | Heptane | 1.61 | 1.11 | 1.45 |
| Example 3 | F:DMF (9:16) | Cyclohexane | 0.57 | 0.39 | 1.48 |
| Example 4 | F:DMF (9:16) | Pentane | 1.54 | 1.13 | 1.36 |
| Example 5 | F:DMF (9:16) | 1-Hexene | 0.53 | 0.39 | 1.37 |
| Example 6 | F:DMF (1:1) | Dodecane | 1.08 | 0.78 | 1.39 |
| Example 7 | F:MF (1:1) | Hexane | 0.52 | 0.44 | 1.20 |
| Example 8 | F:MF (2:3) | Hexane | 1.41 | 1.15 | 1.23 |
| Example 9 | F:DMF:DBF | Hexane | 1.04 | 0.75 | 1.39 |
| | (1:3:1) | | | | |

The cannabinoid sample was exposed to various liquid mixtures in Examples 1-9. The liquid mixtures in Examples 1-6 and 9 exhibited efficient separation of the delta-9-tetrahydrocannabinol and delta-8-tetrahydrocannabinol. The liquid mixtures in Examples 7-8 also demonstrated separation of the delta-9-tetrahydrocannabinol and delta-8-tetrahydrocannabinol, but the separation was less efficient than those observed Examples 1-6 and 9. As shown in Example 9, efficient separation can be obtained using a water soluble phase containing several amide-containing liquids (e.g., such as three amide-containing liquids).

As shown in Table 1, a liquid mixture that can be employed to achieve efficient separation may be associated with a certain set of properties. In general, the liquid mixtures were substantially immiscible such that two distinct phases were established.

Furthermore, it was observed that a liquid mixture having relatively high partition coefficient ratio of K_{d9,THC} to K_{d8,THC} (e.g., at least about 1.3) led to especially efficient separation, as shown in Examples 1-6 and 9. The partition coefficient ratio may be used as an indication of whether a liquid mixture can be used for efficient separation of components in a mixture. For example, a relatively high partition ratio indicates a higher selectivity for delta-9-tetrahydrocannabinol relative to delta-8-tetrahydrocannabinol, and thereby a more efficient separation. A liquid mixture having a relatively low partition coefficient ratio may be less efficient as it may require a more solvent consumption and/or a larger number of extraction stages for separation of the delta-9-tetrahydrocannabinol from the delta-8-tetrahydrocannabinol.

Additionally or alternatively, a liquid mixture that can be used for efficient separation can have a K_{d9,THC} of above 1 and a K_{d8, THC} of below 1. In some cases, it may be advantageous to use liquid mixtures that have the ability (or can be adjusted to have a composition) to result in a K_{d9,THC} of above 1 and a K_{d8,THC} of below 1. As an example, for a water soluble phase comprising formamide (F) and dimethyl formamide (DMF), the relative amounts of F to DMF can be adjusted to result in a K_{d9,THC} of above 1 and a K_{d8, THC} of below 1.

The liquid mixtures in Examples 1-6 and 9 may be employed for particularly efficient separation because the liquid mixtures had a relatively high partition coefficient ratio (e.g., at least 1.3), comprised 2 immiscible phases, and could be adjusted to have a K_{d9,THC} of above 1 and a K_{d8,THC} of below 1. The liquid mixtures in Examples 7-8, although less efficient than those in Examples 1-6 (e.g., having a lower partition coefficient ratio), also comprised 2 immiscible phases and may also be employed for separation of delta-9-tetrahydrocannabinol from delta-8-tetrahydrocannabinol.

### Example 10

This example describes the separation of delta-9-tetrahydrocannabinol from cannabinol using a heterogeneous liquid mixture, in accordance with certain embodiments.

A cannabinoid sample rich in cannabidiol (CBD) content (about 90% CBD) relative to tetrahydrocannabinol (THC) content was exposed a liquid mixture. The cannabinoid sample comprised delta-9-tetrahydrocannabinol. The liquid mixtures included an organic phase and a water soluble phase. The organic phase and the water soluble phase were in 1:1 volume ratio for all analyses. A mixture containing formamide (F) and dimethyl formamide (DMF) at a volume ratio of 65% F to 35% DMF was used as the water soluble phase. Hexane was used as the organic phase. The partition coefficient for each component (e.g., delta-9-tetrahydrocannabinol, cannabidiol) in the liquid mixture was determined by calculating a ratio of the concentration of the component in the water soluble phase over concentration of the component in the organic phase. While cannabidiol had a partition coefficient K_{CBD} of 1.87, delta-9-tetrahydrocannabinol had a partition coefficient K_{d9,THC} of 0.4. The liquid mixture resulted in efficient separation of delta-9-tetrahydrocannabinol from cannabidiol.

### Example 11

This example describes an embodiment of a multi-stage liquid-liquid extractive chromatographic system, according to some embodiments.

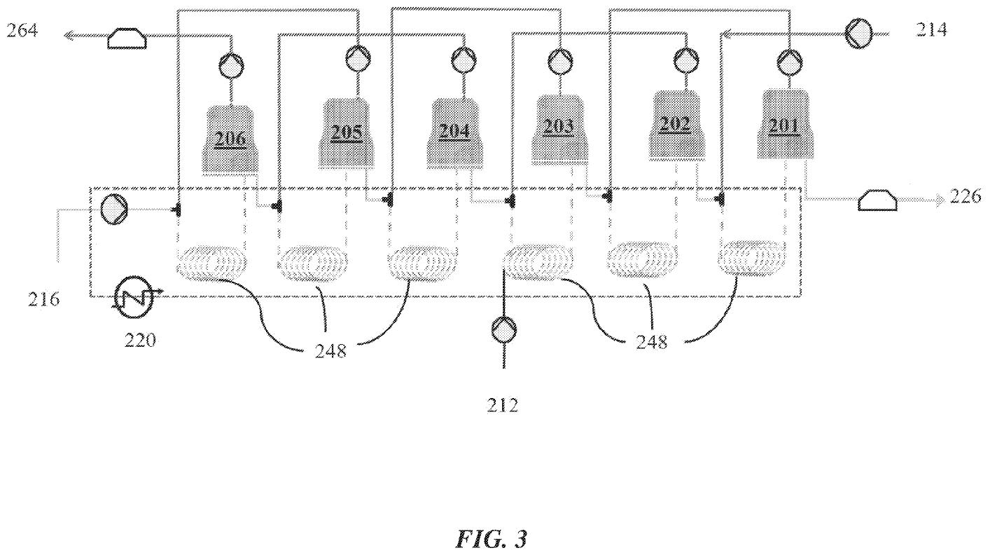

A membrane-assisted multi-stage liquid-liquid extractive chromatographic system (e.g., as shown in FIG. 3) comprising a biphasic extraction liquid system is described. The countercurrent chromatographic system described herein is fully continuous, e.g., such that the sample to be separated can be injected continuously into the system rather than in discrete quantities. The system may be able to separate a group of chemical species from another using the biphasic extractive liquid system. The chemical species may be separated based on the difference in their respective elution times, and the elution times may be dependent on the value of the partition coefficients the chemical species have in the biphasic liquid extractive liquid system.

As shown in FIG. 3, the liquid-liquid extractive chromatographic system may comprise a modular structure comprising 6 modular separator stages. Contrary to typical chromatographic systems that employ a mobile phase and a stationary phase, two mobile immiscible phases (e.g., first phase liquid 214 and second phase liquid 216) may be employed in this extractive chromatographic system and flow in a countercurrent fashion in the system. A feed liquid stream (e.g., feed liquid stream 212) comprising a mixture of solute A and solute B may be fed into one of the intermediate separator stages (e.g., intermediate separator stage 203). The two mobile immiscible phases may function as extraction liquids that can be used to extract and separate solute A and solute B in the feed liquid stream.

At each stage, the two mobile immiscible phases may be mixed outside the separation chamber (e.g., separation chambers 201, 202, 203, 204, 205, 206), using either dynamic or static (active or passive) mixing at various mixing regions (e.g., mixing regions 248), before entering the separation chamber as a mixed liquid stream comprising the two immiscible mobile phases and solutes A and B. During the dynamic or static mixing, solute A may preferentially associate with first phase liquid 214 and solute B preferentially associates with the second phase liquid 216 based on a difference in their partition coefficients. For example, while solute A may have a partition coefficient KA between the first liquid phase and second liquid phase of greater than 1, solute B may have a partition coefficient KB between the first liquid phase and the second liquid phase of less than 1. Inside each separation chamber, phase separation between the mixed liquid stream may carried out via membrane-based separation technology using an integrated pressure controller. A liquid comprising pure solute A solubilized in the first phase liquid (e.g., liquid 264) may be produced and a liquid comprising pure solute B solubilized in the second phase liquid (e.g., liquid 226) may be produced.

The system may include sensors, storage space and pumps in between each stage in order to store, pressurize and send upstream the phase moving in countercurrent direction. The feed liquid stream comprising the mixture of solutes may be inserted and mixed at a mixing region before any stage in the unit. The positioning of the feed liquid inlet for receiving the feed liquid stream may vary depending on the objectives of the separation process. The temperature of the biphasic liquid mixture throughout the system (e.g., associated with the mixing regions 248) may be controlled using a temperature control system (e.g., temperature control system 220). Overall, the system described herein may allow for facile modification of solvent composition or pH, and temperature throughout the operation. One or more in-line analytical measurements (e.g., IR or Raman measurements) may be employed to monitor the above-referenced physical properties.

### Example 12

This example describes using a multi-stage liquid-liquid extractive chromatographic system for separation of various compounds, according to some embodiments. Experiments were performed using the system described herein to measure the extraction efficiencies of various compounds, as described below.

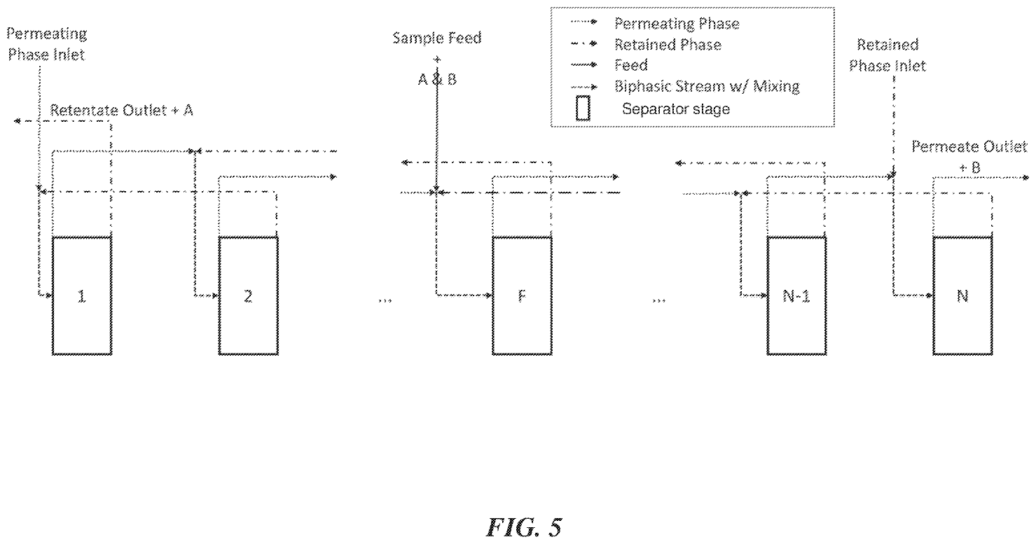

FIG. 5 is a schematic diagram of a multi-stage liquid-liquid extractive chromatographic system (i.e., multi-stage separator extraction system). The system may be capable of utilizing differences in wetting properties to separate biphasic solvent systems and includes a countercurrent scheme with an intraplatform feed injection. The system, when in this orientation, may allow for neat immiscible solvents to enter the platform through stages at the opposite extremities of the system and the feed compounds (which are dissolved either in one of the phases or a solvent miscible with only one of the phases) to enter the platform before one of the intermediate stages. The versatility of the multi-stage extraction platform makes it such that stages can easily be added or removed as needed and the location of the feed stage can be easily changed to meet process recovery and purity requirements.

A schematic of a theoretical setup with N extraction stages connected in a countercurrent manner with the feed entering the system at stage F containing compounds A and B is shown in FIG. 5. As shown, Compound A favors the retained phase and will travel toward the retentate outlet, while Compound B favors the permeating phase and will travel towards the permeate outlet.

The system shown in FIG. 5 involves the use of two immiscible liquid phases and can result in 100% sample recovery. Additionally, the system has many advantages compared to other conventional systems. First, it is cost effective compared to HPLC and SMB (simulated moving bed chromatography) as it can be used with lower purity solvents and lacks expensive solid supports. Since the wetted parts of the separator stages can be made of fully perfluorinated polymers, there will be no pH limitations for operating the equipment.

The separator stages have also been proven to be easily scalable with no loss in extraction efficiency. For example, when the phases entering the separator are at equilibrium, equivalent extraction efficiencies can be achieved across different scales. During operation, it was observed that the equipment also had a minimal pressure drop across the system when compared to CPC (centrifugal partition chromatography) and HPLC, with each separator stage experiencing less than 0.5 bars of pressure drop. Additionally, there are little to no limitation on the minimum/maximum K_{D} values the compounds of interest need to have to allow for adequate separation resolution as is the case with CPC. In fact, it was observed that the farther a partition coefficient for a given compound is from another compound, the higher the achievable separation of those two compounds for a given number of stages. When necessary, additional stages can be added to further increase purity and recovery. K_{D} and α are defined as follows:

$${K}_{D} = \frac{{[ solute ]}_{organic}}{{[ solute ]}_{aqueous}} \qquad ( 1 )$$

$$α = \frac{{K}_{D , compound 1}}{{K}_{D , compound 2}} \qquad ( 2 )$$

Furthermore, since both liquid phases may be continuously flowed into and out of the platform, there is also little to no possibility for compounds to become trapped in the equipment should it have a high affinity for the immobilized phase as with CPC. The platform also includes no external valves, switches, rotors, or seals which need to be monitored or repaired, which negates the need for expensive maintenance and the possibility of electrical failure. Additionally, since the separators have been proven to be fully density independent, this can greatly expand the number of solvent systems available for use and allow for many new solvent combinations to be used for a given system. Finally, the system may allow for continuous operation to achieve high throughput purification, even at the laboratory scale. For example, depending on the concentration of compounds in the feed stream and ratio of feed stream to neat solvent stream, multiple grams of sample compounds can be separated per hour.

### Example 12A: Separation of Aldehydes

4-ethylbenzaldehyde (4-EBA) and cuminaldehyde (CA) were chosen as compounds to be separated as these compounds both have K_{D} values close to 1 when separated using a system such as a 5/4/1 hexane/methanol/water system. Additionally, cuminaldehyde (CA) has been found to partition similarly to one of the major cannabinoids using this solvent system. As the global legal cannabis market is projected to rapidly expand, the ability to purify individual cannabinoids (and/or similar compound(s)) may be of interest. These factors combine to make this an interesting example for determining the performance of the system described herein for the separation of cannabinoids.

### Example 12B: Separation of Depolymerized Lignin Components

Experiments directed to separation of a natural product centered around some of the major components from the depolymerization of softwood lignin was performed. Softwood lignin and its depolymerized components have recently attracted attention for its economic optimization and valorization through its use in biorefining and applicability as a renewable biomass to use as a starting material in the polymer industry.

### Example 12C: Separation of Chiral APIs

Finally, an experiment directed to enantioseparation of racemic propranolol, a β-adrenergic receptor blocker drug, using a chiral selector, was performed. Recently, there has been increasing interest in using additives which preferentially bond with one enantiomer to skew partitioning through a three-point interaction model. Due to the inherent mirror plane found in chiral compounds, one of the enantiomers will be able to interact with a chiral selector (e.g., be it through hydrogen bonding, van der Waals forces, steric hinderance, etc.) in three locations, while the opposite enantiomer will only be able to interact in two, thus the selector will preferentially favor the interaction with one of the enantiomers and skew the partition of that enantiomer into the phase containing the selector. Common chiral selectors may include β-cyclodextrin derivatives, chiral amine derivatives, and tartaric acid derivatives.

### Experimental Procedures for Examples 12A-12C

Water was HPLC grade and purchased from Honeywell (North Carolina, USA). HPLC grade methanol, analytical reagent grade n-hexane, 95% lab grade hexanes, and 98% lab grade methanol were all purchased from Lab Alley (Texas, USA). LCMS grade 25% ammonia as well as analytical reagent grade ethyl acetate, ammonium formate, 4-ethylbenzaldehyde, cuminaldehyde, dichloromethane, glacial acetic acid, boric acid, and R-propranolol HCl were all purchased from Millipore-Sigma (Missouri, USA). Vanillic acid, para-hydroxybenzoic acid, syringaldehyde, rac-propranolol HCl, and dibutyl L-(+)-tartrate were all analytical reagent grade and purchased from TCI (Pennsylvania, USA).

For all HPLC analysis, an Agilent 1100 series (Agilent Technologies Corporation, USA) equipped with a G1314A variable wavelength detector and an Xbridge BEH C₁₈ column (150 mm×2.1 mm ID, 3.5 μm) (Waters, USA) was utilized. The specifics of each method can be found below.

Aldehydes. The mobile phases consisted of 40% 10 mM ammonium formate in water adjusted to a pH of 10.2 using 25% ammonia as Mobile Phase A and 60% acetonitrile as Mobile Phase B at a flow rate of 0.65 mL/min. The column was heated to 25° C. and the detection was monitored at a wavelength of 270 nm. Samples were diluted 75× in ethyl acetate and the injection volume was 1 μL.

Lignin Depolymerization. The mobile phases consisted of 0.1% formic acid in water as Mobile Phase A and acetonitrile as Mobile Phase B at a flow rate of 0.6 mL/min. Linear gradient elution was performed using the following profile: 0.0-0.5 mins, 5% B; 0.5-5.0 mins, 7% B; 5.0-10.0 mins, 9% B; 10.0-10.5 mins, 5% B; and 10.5-15.0 mins, 5% B. The column was heated to 35° C. and the detection was monitored at a wavelength of 280 nm. Samples were diluted 10× in methanol and the injection volume was 10 μL.

Chiral API. One mobile phase was used—a 54/46 v/v methanol/water solution containing 110 mM sodium acetate adjusted to a pH of 6 using glacial acetic acid, 215 mM boric acid, and 285 mM dibutyl L-(+)-tartrate. The flow rate was 0.23 mL/min. The column was heated to 25° C. and the detection was monitored at a wavelength of 275 nm. Samples were diluted 20× in 54/46 v/v methanol/water solution containing 110 mM sodium acetate buffered to a pH of 6 with glacial acetic acid and the injection volume was 10 μL.

### Extraction Methods

Aldehydes. Batch extraction experiments were performed as follows. A known amount of 4-ethylbenzaldehyde and cuminaldehyde was dissolved in hexane to bring the total concentration of both species to between 10-40 mg/mL. 2 mL of this organic solution weas combined with 2 mL of 80/20 v/v methanol/water mixture and vigorously shaken for around 2 minutes and allowed to fully settle. 20 μL of each phase were diluted with 1480 μL of ethyl acetate and the concentrations were determined by HPLC.

Multi-stage separation experiments were performed using either a Zaiput MS10-5 five-stage or MS10-10 ten-stage multi-stage extraction platform fitted with OB-900 membranes (e.g., PTFE hydrophobic membranes with average pore diameters of 0.9 micrometers). The total working volumes of the platforms were about 15 mL and about 30 mL, respectively. The feed stage, ratio of feed to fresh organic, and aqueous/organic outlet phase ratio were all varied to simulate possible working conditions.

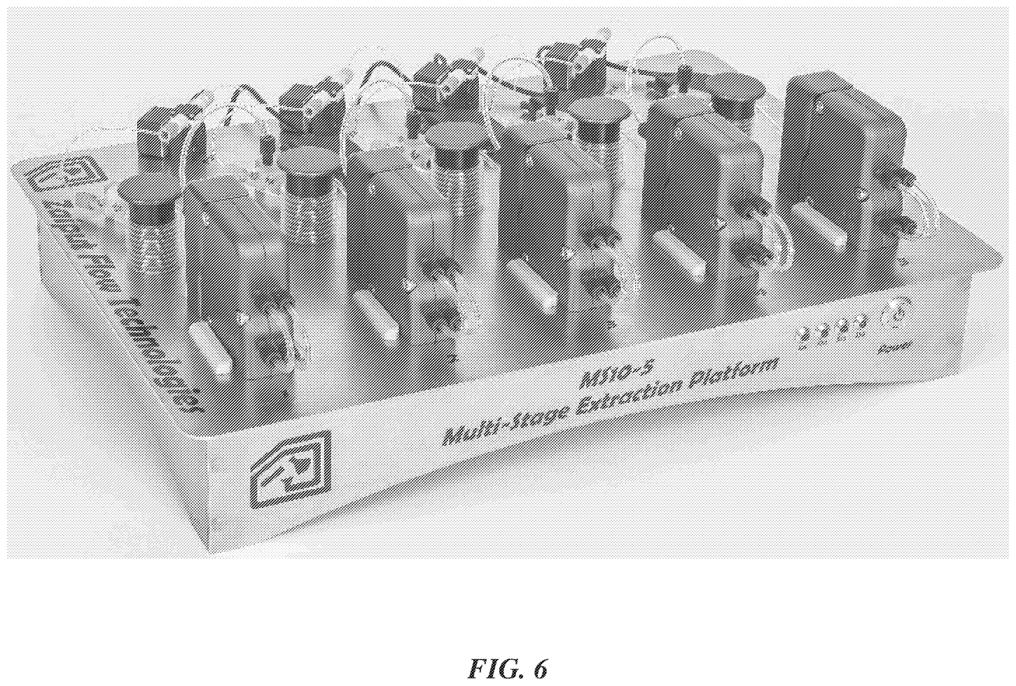

When using the MS10-5 platform, the feed stage was varied from 1-5; the ratio of feed to fresh organic was either 1/4, 1/1, or 4/1; and the outlet phase ratios of the aqueous and organic phases was either 1/3, 1/1, or 3/1. When using the MS10-10 platform, the feed stage was either 2, 4, 6, or 8; the ratio of feed to fresh organic was either 1/4, 1/1, or 4/1; and the outlet phase ratios of the aqueous and organic solvents were either 1/3, 1/1, or 3/1. In all experiments, the feed was dissolved in hexane and each solute had a concentration of 50 mg/mL and the total flow rate was 5 mL/min. Samples of the aqueous and organic phases were taken after 12 residence times to ensure the platform was at steady state and diluted with ethyl acetate as described above before being analyzed by HPLC. A photo of the 5-stage multi-stage extraction platform is shown in FIG. 6.

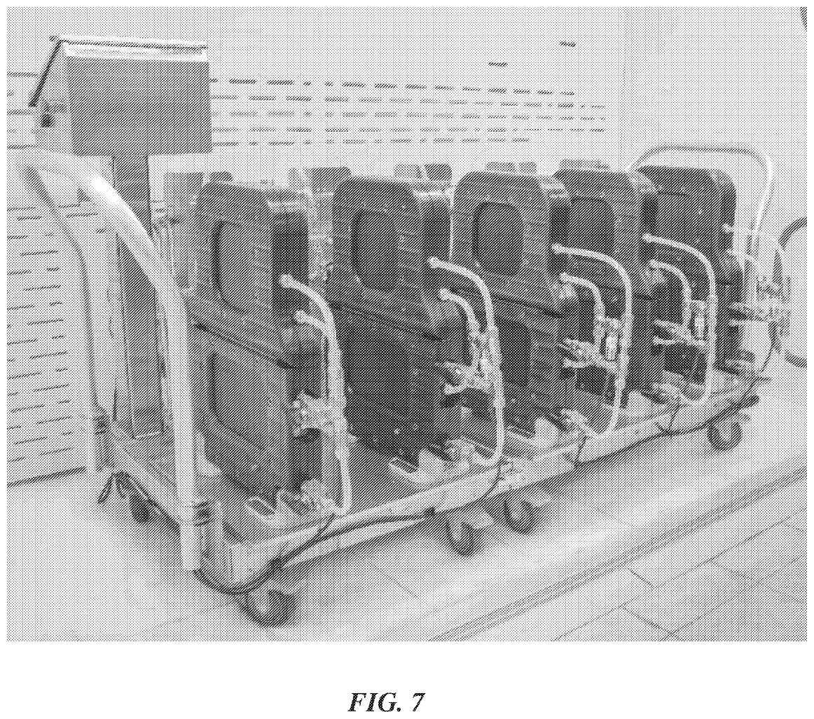

Aldehyde Scale-Up. Separation experiments were also able to be performed on a 5-stage pilot plant scale of the multi-stage extraction platform capable of flow rates up to 3 L/min-fitted with OB-900 membranes. The working volume of the platform was approximately 3 L. Stage 3 was used as the feed stage, the feed was dissolved in the organic phase at a concentration of 50 mg/mL for each component, the ratio of feed to fresh organic was 1/4, the phase ratio at the aqueous and organic outlets was 2.76/1, and the total flowrate was 750 mL/min. A photo of the 5-stage pilot plant scale platform is shown in FIG. 7.

Lignin Depolymerization. Batch extraction experiments were performed as follows. 1 mg of each solute was added to 2 mL of dichloromethane, 1 mL of water, and 1 mL of methanol. The solution was vigorously shaken for around 2 minutes and allowed to fully settle. 100 μL of each phase were diluted with 900 μL of methanol and the solute concentrations were determined by HPLC.

Multi-stage extraction experiments were performed using a Zaiput MS10-10 multi-stage extraction platform fitted with OB-400 membranes (e.g., PTFE hydrophobic membranes with average pore diameters of 0.2 micrometers). The total working volume of the platform was about 30 mL. The feed was injected into the system before stage 4 and the following flow rates were used: 2.25 mL/min of fresh organic, 2.2 mL/min of fresh aqueous, and 0.55 mL/min of aqueous feed with a concentration of about 2.27 mg/mL. This feed concentration was used to bring the global concentration of the platform to about 1 mg of each solute for every 4 mL of solvent as used in the batch extraction experiments. Samples of the aqueous and organic phases were collected every residence time for 10 residence times and diluted with methanol as described above before being analyzed by HPLC.

Chiral API. Batch extraction experiments were performed as follows. 2 mL of 1 mg/mL rac-Propranolol in water containing 152.1 mM boric acid was mixed with 2 mL of 152.1 mM dibutyl L-(+)-tartrate in dichloromethane. The solution was vigorously shaken for approximately 2 minutes and allowed to fully settle. 20 μL of each phase were diluted with 380 μL of 110 mM sodium acetate in a 54/46 methanol/water solution buffered to a pH of 6 using glacial acetic acid and the solute concentrations were determined by HPLC.

Multi-stage extraction experiments were performed using a Zaiput MS10 multi-stage extraction platform fitted with ILG-900 membranes (e.g., hydrophilic PTFE membranes with 0.5 micron pore size). The total working volume of the platform was about 30 mL. The feed was injected into the system before stage 5 and the following flow rates were used: 2.058 mL/min of fresh organic, 0.353 mL/min of fresh aqueous, and 0.088 mL/min of aqueous feed with a rac-Propranolol HCl concentration of 5 mg/mL. This feed concentration was used to bring the average concentration of platform to 1 mg per mL of aqueous phase as used in the batch extraction experiments. Samples of the aqueous and organic phases were collected every residence time for 10 residence times and diluted with a sodium acetate buffer as described above before being analyzed by HPLC.

### Results and Discussion

### Example 12A-Separation of Aldehydes

The distribution coefficients of 4-ethylbenzaldehdye and cuminaldehyde were determined via batch extraction at concentrations of 10, 25, and 40 mg/mL corresponding to 1/4, 1/1, and 4/1 feed to fresh organic ratio—the resulting dilutions of the 50 mg/ml feed at a given feed to organic ratio. The distribution coefficients at each concentration can be found in Table 2.

TABLE 2
Distribution coefficients based on concentration for
4-ethylbenzaldehyde and cuminaldehyde.

| | Corresponding Feed | | |
|---|---|---|---|
| Concentration | to Fresh Organic | | |
| [mg/mL] | Ratio | K_{D, 4-EBA} | K_{D, CA} |
| 10 | 1/4 | 0.98 | 1.27 |
| 25 | 1/1 | 1.02 | 1.37 |
| 40 | 4/1 | 1.15 | 1.58 |

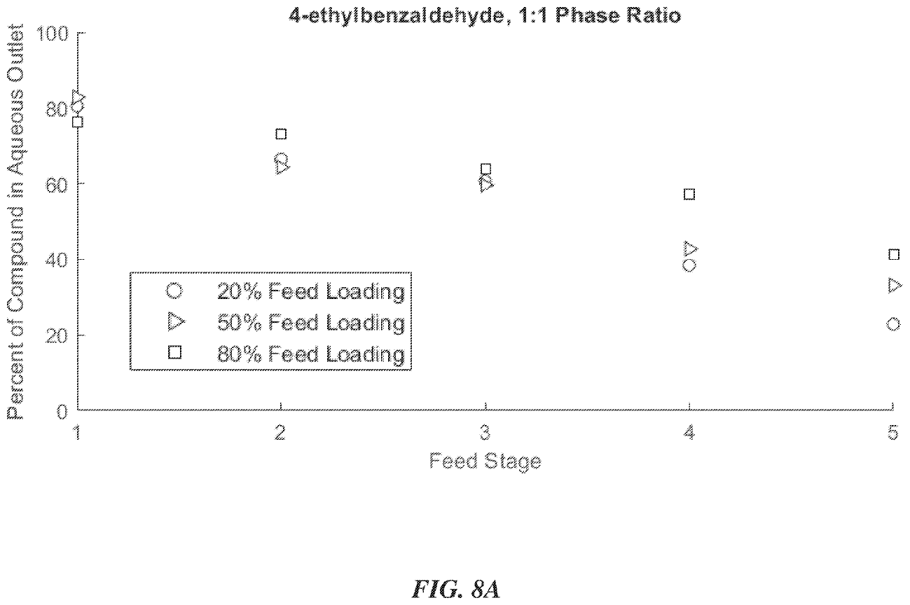

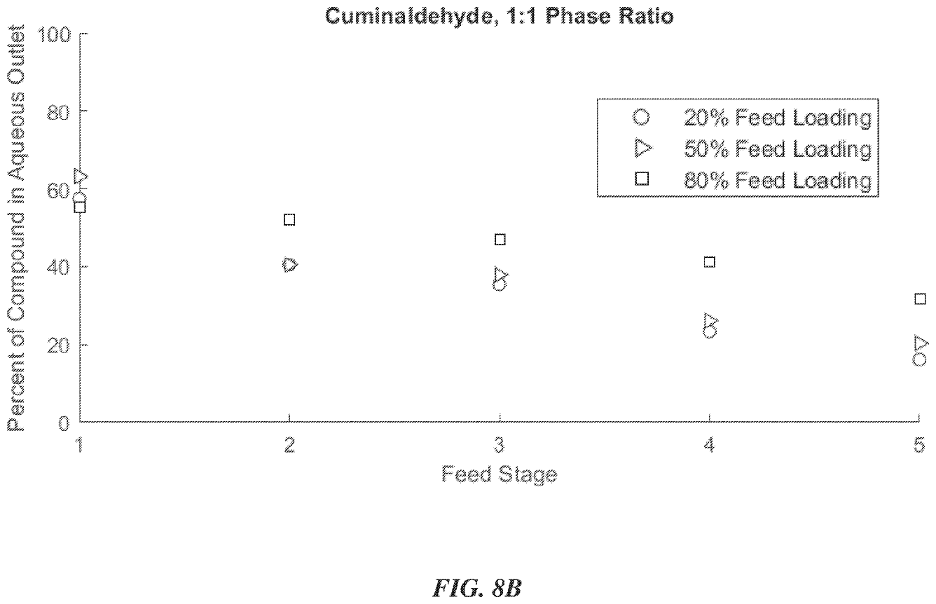

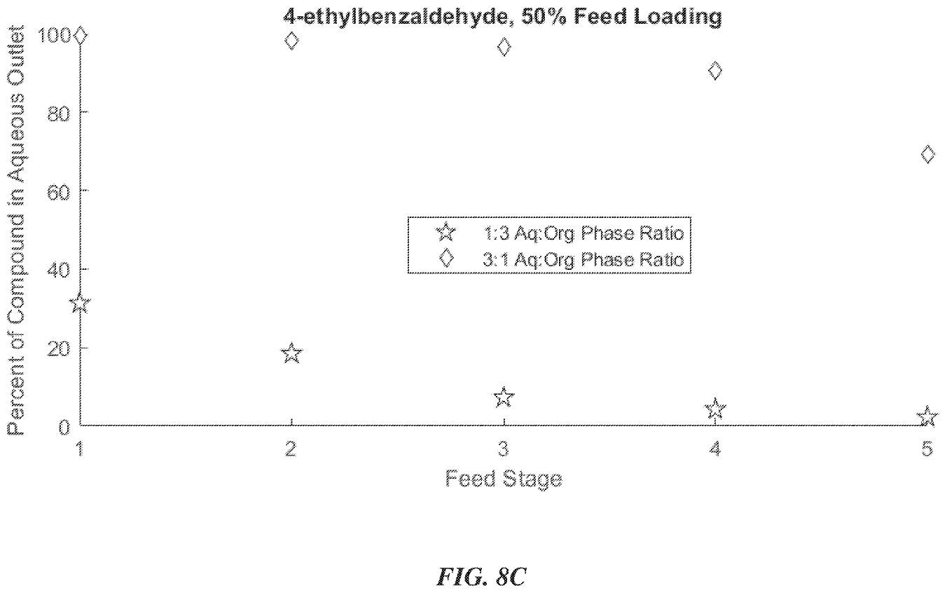

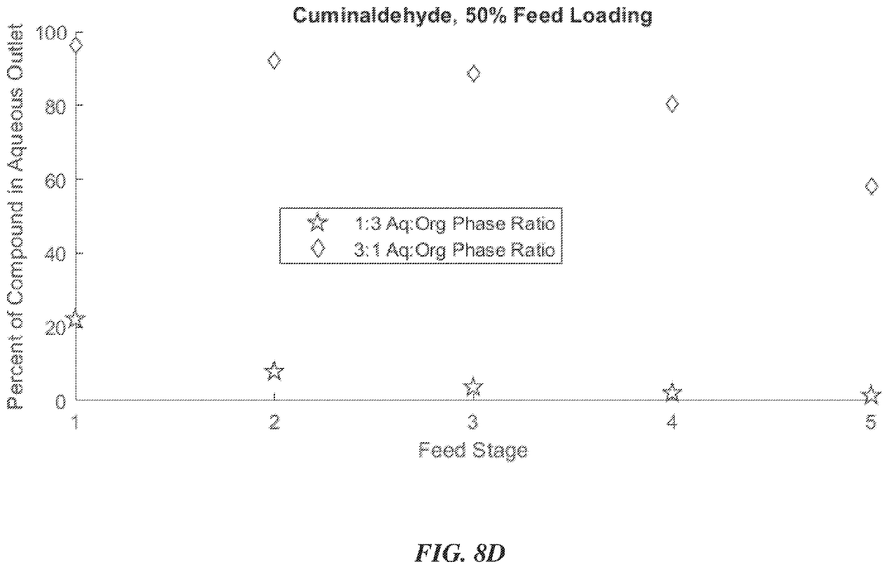

The experimental separation efficiencies for a given feed to organic ratio and feed stage location at a 1/1 aqueous to organic phase ratio using the 5-stage extraction platform are shown in FIGS. 8A-8B.

The experimental separation efficiencies for a given aqueous to organic phase ratio and a given feed stage location at a 1/1 feed to fresh organic ratio are presented in FIGS. 8B-8D.

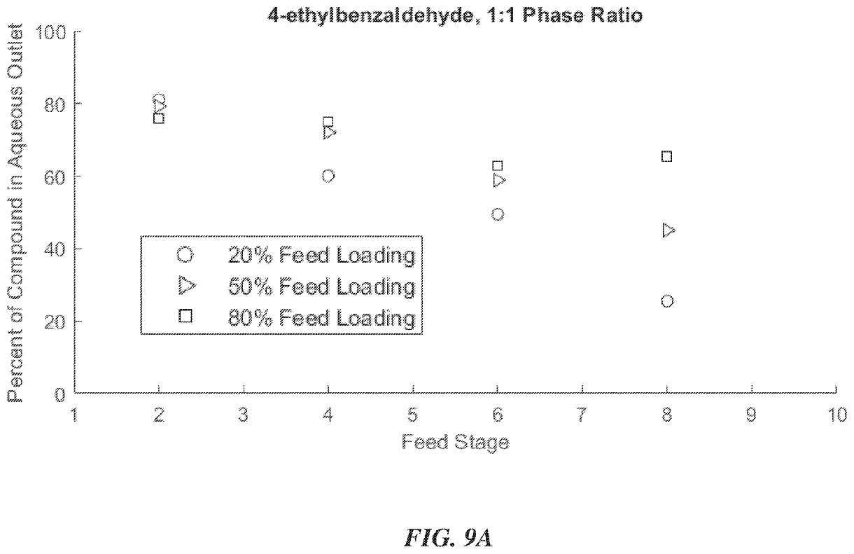

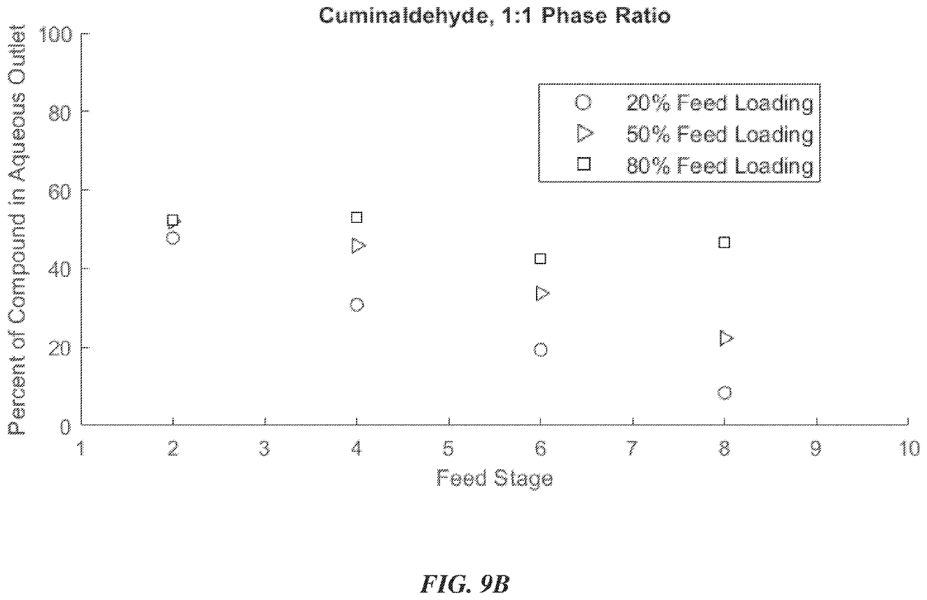

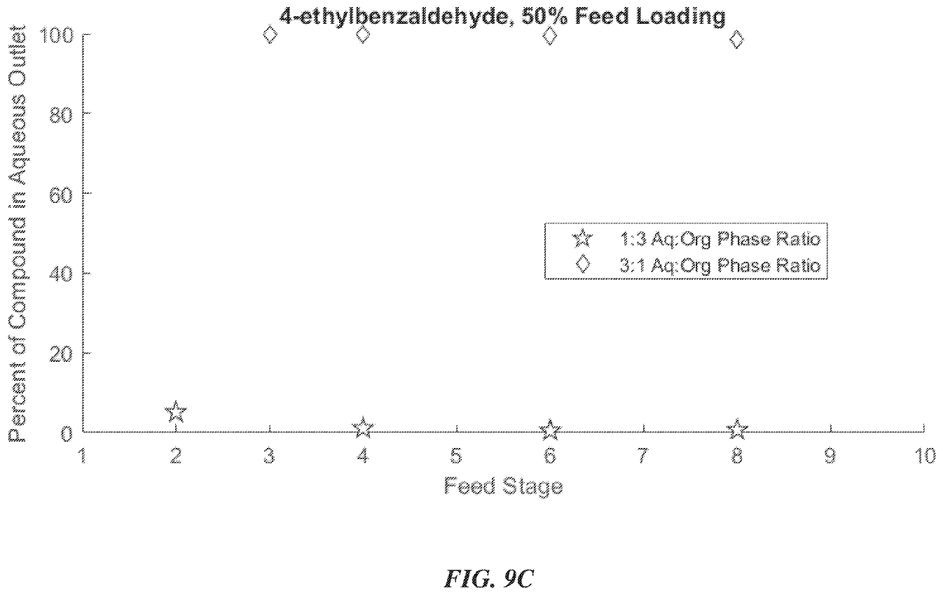

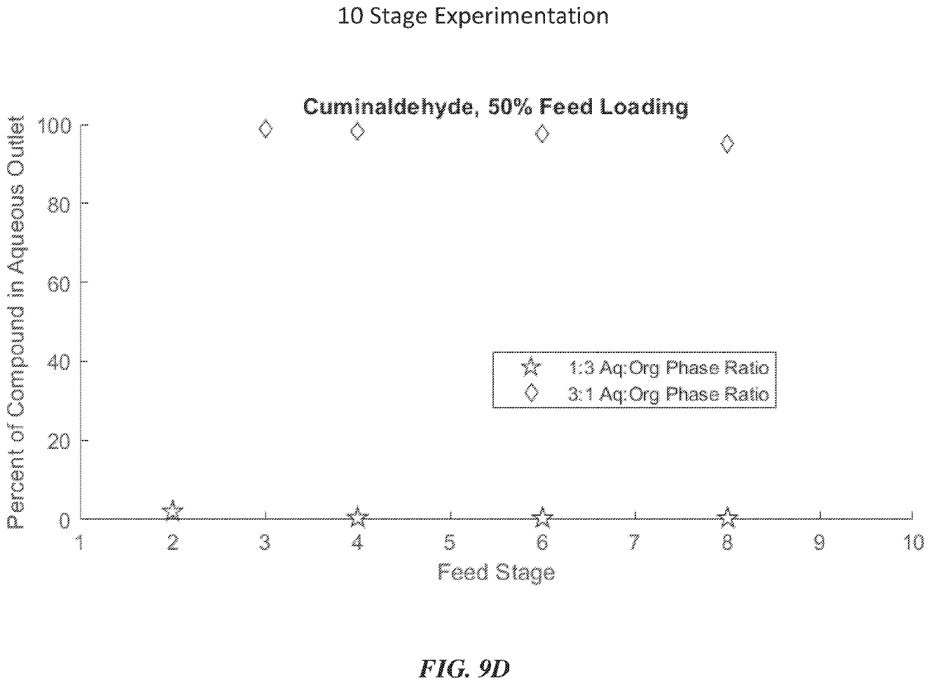

Similarly, the experimental separation efficiencies for a given feed to organic ratio and varying feed stage location at a 1/1 aqueous to organic phase ratio using the MS10-10 multi-stage extraction platform are shown in FIGS. 9A-9D.

### Aldehydes Scale-Up

For testing at the pilot scale, 95% hexanes and 98% methanol were purchased for testing. Due to this difference in grade, the K_{D} values were remeasured for pilot scale experiments and compared to the results found using reagent grade hexane and HPLC grade methanol at the lab scale. The new partition coefficients can be found in Table 3.

TABLE 3
Comparison of K_{D} values for different hexane and methanol grades with 4-
ethylbenzaldehyde and cuminaldehyde concentrations of 10 mg/mL.

| Compound | K_{D} at Lab Scale | K_{D} at Pilot Scale |
|---|---|---|
| 4-ethylbenzaldehyde | 0.98 | 3.21 |
| Cuminaldehyde | 1.27 | 1.97 |

As can be seen in the table, reducing the purity of the solvents resulted in both of the compounds beginning to increasingly favor the organic phase, likely due to the impurities present in the hexane. A 2.75/1 aqueous to organic outlet phase ratio with a 1/4 feed to organic ratio and the feed entering at stage 3 were used for separation.

TABLE 4
Experimental percentage of each compound extracted into the
aqueous phase on the pilot scale multi-stage extraction system.

| | Experimental Extraction |
|---|---|
| Compound | Efficiency |
| 4-ethylbenzaldehyde | 43.8 |
| Cuminaldehyde | 58.8 |

These experiments show the capability of technology and method to be easily scaled from laboratory scale to a pilot or production plant.

### Example 12B-Lignin Depolymerization

In batch experiments, a 10/6/4 DCM/methanol/water ratio has been traditionally used as it gave K_{D} values near the generally accepted range of 0.4 to 2.5 and had small settling time which allowed for the compounds to elute with high resolution without staying in the column for an excessive period of time and consuming excess solvent. However, since the platforms described herein lacks an immobilized phase and continuously flows both phases into and out of the platform, solute retention is no longer an issue and the bigger the difference between K_{D} values, the better it is for separation efficiency. It was observed that a 10/5/5 DCM/methanol/water ratio gave higher alpha values between as seen in Table 5.

TABLE 5
Comparison of distribution coefficients and selectivity
between the experiments with a 10/5/5 DCM/methanol/water
ratio and those from literature with a 10/6/4 ratio.

| | Literature | | Current platform | |
|---|---|---|---|---|
| | (10/6/4 ratio) | | (10/5/5 ratio) | |
| Compound | K_{D} | α_{x/pHBA} | K_{D} | α_{x/pHBA} |
| pHBA | 0.23 | 1.00 | 0.18 | 1.00 |
| Vanillic Acid | 0.50 | 2.21 | 0.49 | 2.77 |
| Syringaldehyde | 4.05 | 17.8 | 4.51 | 25.5 |

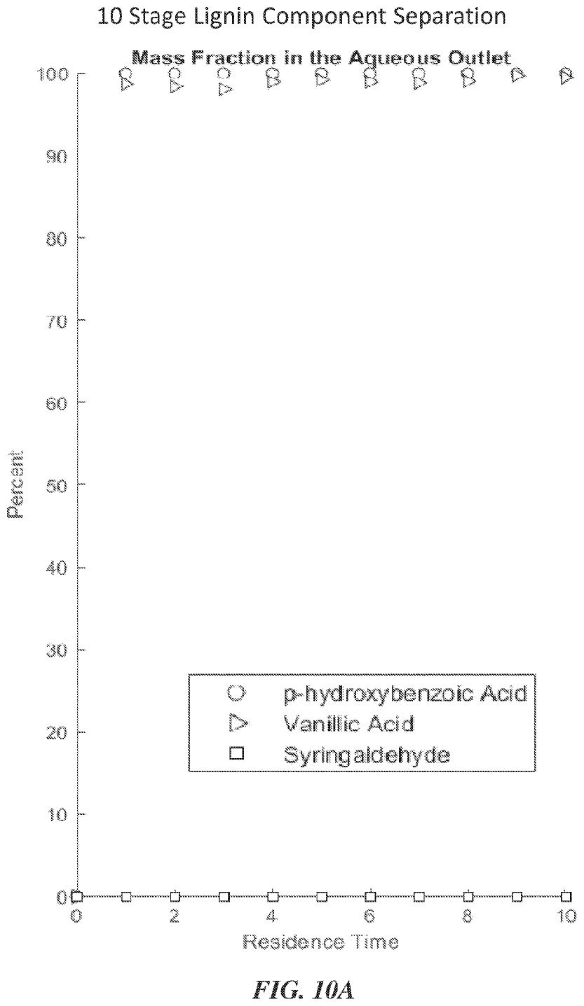

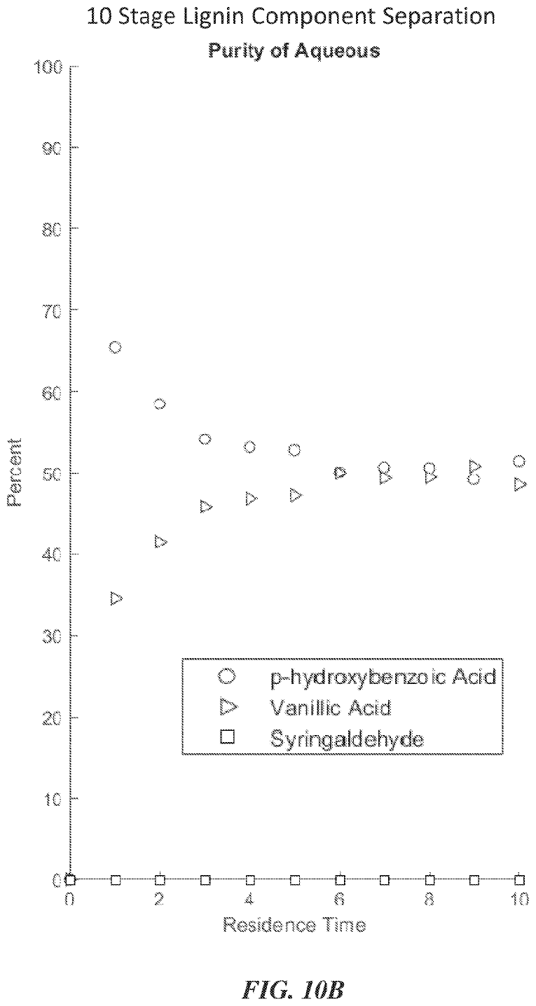

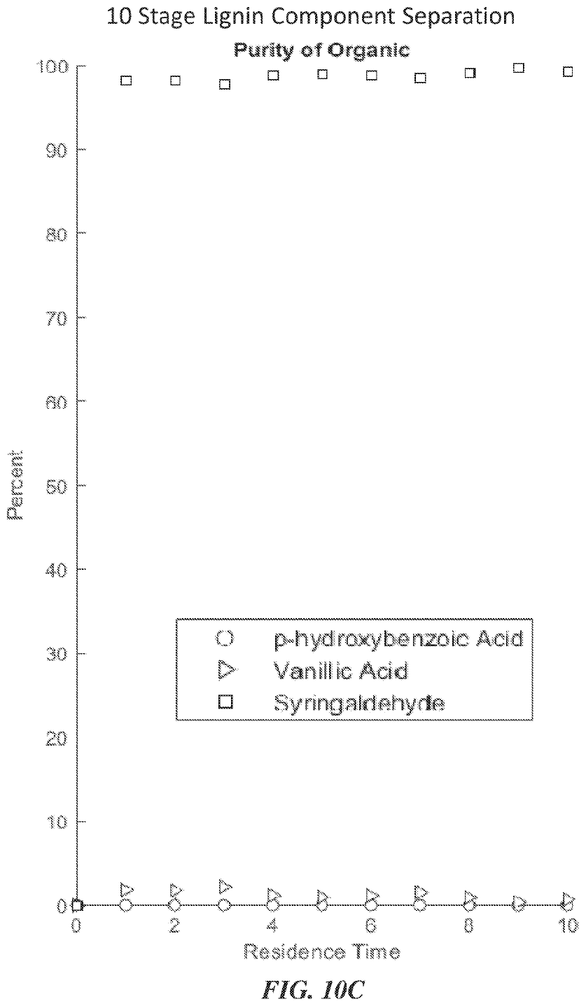

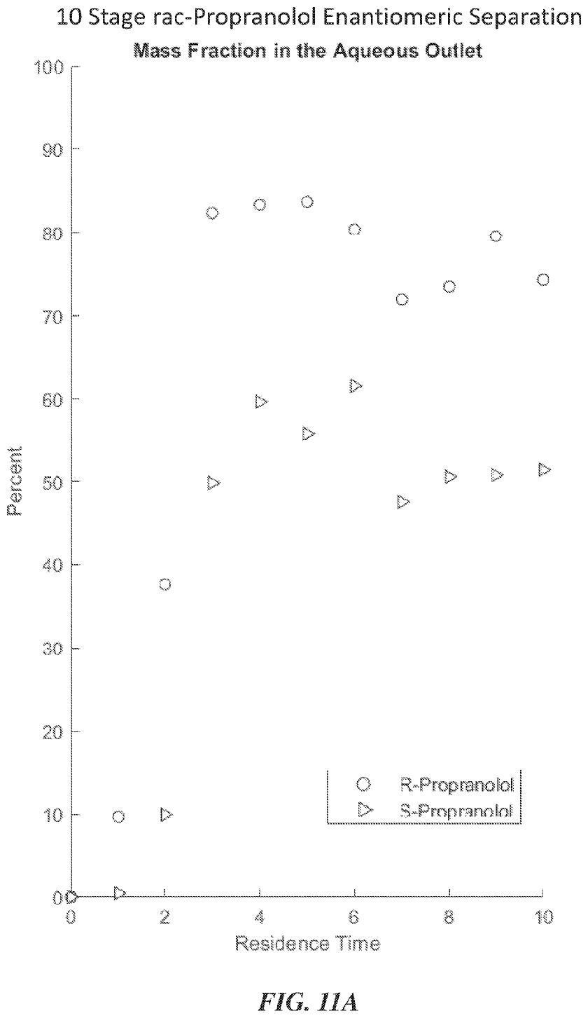

Experimental extraction efficiencies and outlet purities of the separation of compounds present in depolymerized lignin are shown in FIGS. 10A-10C.

As shown in FIGS. 10A-10C, the separation takes about 6 RT to come to steady state in the aqueous phase with the purity of the organic phase coming to steady state almost immediately. Due to the high partitioning of pHBA and syringaldehyde into the aqueous and organic phases respectively, these compounds quickly begin eluting after the start of operation since there is little back extraction of these compounds between phases. As vanillic acid has a moderate affinity for both phases, it would take more time for it to begin fully eluting at the aqueous outlet as it experiences some initial extraction into the organic phase and takes time for it to be fully back extracted into the aqueous phase. After reaching steady state, it was determined that 100% of the syringaldehyde was captured in the organic outlet with 99.3% purity, which demonstrates the equipment's capability to achieve purity and recovery comparable to (if not better than) traditional chromatography techniques.

### Example 12C-Chiral APIs

Enantiomers of R/S-propranolol (R/S-PRP) could be separated by a mixture of 1,2-dichloroethane (DCE) containing dibutyl-L-tartrate (DBLT) and water containing boric acid (BA), both additives in a 45× molar excess to the concentration of R/S-PRP, by forming a ternary complex using BA as an intermediate between DBLT and R/S-PRP. Using this solvent system, the enantiomers could be separated with a selectivity of 2.54±0.13. However, due to the increased toxicity of DCE, it was substituted with dichloromethane (DCM) as it had been noted to partition R/S-PRP with equivalent selectivity to DCE. Performing batch experiments replacing DCE with DCM was found to give almost identical selectivity as shown in Table 6.

TABLE 6
Comparison between selectivity between the experiments
conducted in this Example using dichloromethane as
the organic phase and those in literature, using 1,2-
dichloroethane as the organic phase. Both experiments
had a 45x molar excess of dibutyl-L-tartrate to
R/S-propranolol in the organic phase and
used water containing an equivalent
molar excess of boric acid as the aqueous phase.

| | 1,2-Dichloroethane/water | Dichloromethane/water |
|---|---|---|
| | (literature) | (experiment) |
| Selectivity | 2.54 ± 0.13 | 2.58 ± 0.07 |

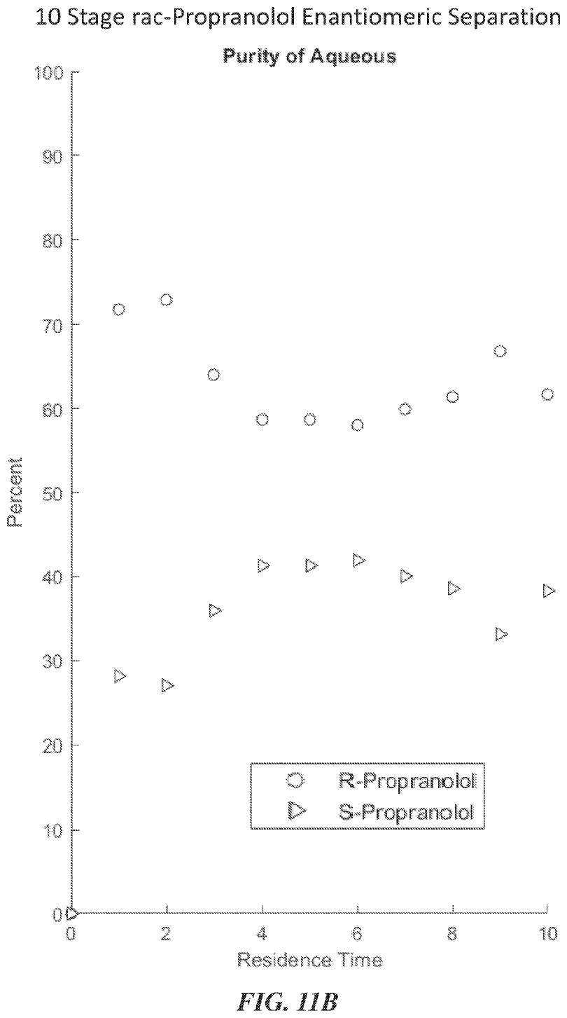

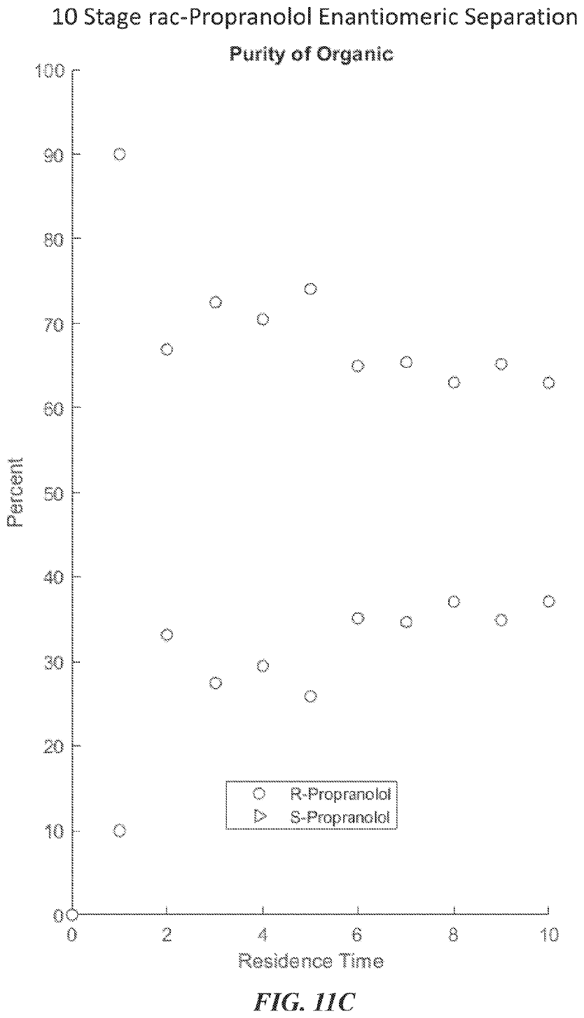

Using this solvent system, the distribution coefficients for R-PRP and S-PRP were determined to be 0.12 and 0.30, respectively. A 4.66/1 organic to aqueous outlet phase ratio with 20% of the aqueous feed being injected into the system at stage 5 was used. Experimental extraction efficiencies and outlet purities for the separation of R/S-propranolol are illustrated in FIGS. 11A-11C.

As shown in FIGS. 11A-11C, despite the large selectivity between the enantiomers and high predicted extraction efficiency, the steady state of the system resulted in 75.9% recovery and 61.6% purity of R-PRP in the aqueous phase and 47.8% recovery and 64.3% purity of S-PRP in the organic phase, all of which could be further increased by the addition of more separation stages. This extraction efficiency is consistent with that of others for this separation.

This Example describes platforms and methodologies for the continuous purification and separation of analytes using a continuously injected feed into a biphasic system of immiscible liquid phases traveling counter-currently through membrane-based phase separators. This equipment closes the gap between the continuous purification capable with simulated moving bed chromatography and the low cost of operation and ease of scalability offered by centrifugal partition chromatography and is a significant step toward achieving true moving bed chromatography. Its effectivity was demonstrated with the complete removal of syringaldehyde from other components of the depolymerization of softwood lignin. Additionally, the availability of this equipment for use in the separation of chiral compounds was demonstrated through the continuous enantioseparation of R/S-propranolol. Due to the lack of an expensive solid phase, modularity of stages, ease of scalability with no loss in separation efficiency, and ease of adaptability of operating conditions to meet process specifications, this equipment can serve as an attractive alternative for the chromatographic separation of compounds in solution.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-34. (canceled)

35. A liquid-liquid chromatographic separator system, comprising:
three or more separator stages, wherein the three or more separator stages are arranged in series with one another from a first separator stage to a last separator stage, with one or more intermediate separator stages positioned between the first separator stage and the last separator stage, wherein each of the three or more separator stages comprises a liquid inlet and two liquid outlets; and
a feed liquid inlet configured to receive a feed liquid stream comprising a first solute and a second solute;
wherein:
the first separator stage comprises:
a first liquid inlet configured to receive liquid comprising at least a portion of the first solute and at least a portion of the second solute,
a liquid outlet configured to output a liquid having a mole fraction of the first solute relative to the sum of the first solute and the second solute that is larger than a mole fraction of the first solute relative to the sum of the first solute and the second solute in the liquid received by the first liquid inlet of the first separator stage, and
a liquid outlet configured to output a liquid having a mole fraction of the second solute relative to the sum of the first solute and the second solute that is larger than a mole fraction of the second solute relative to the sum of the first solute and the second solute in the feed liquid stream; and
the last separator stage comprises:
a last liquid inlet configured to receive a liquid comprising at least a portion of the first solute and at least a portion of the second solute,
a liquid outlet configured to output a liquid having a mole fraction of the first solute relative to the sum of the first solute and the second solute that is larger than a mole fraction of the first solute relative to the sum of the first solute and the second solute in the feed liquid stream, and
a liquid outlet configured to output a liquid having a mole fraction of the second solute relative to the sum of the first solute and the second solute that is larger than a mole fraction of the second solute relative to the sum of the first solute and the second solute in the liquid received by the last liquid inlet.

36. The liquid-liquid chromatographic separator system of claim 35, wherein the three or more separator stages are liquid-liquid phase chromatographic separator stages.

37. The liquid-liquid chromatographic separator system of claim 35, wherein:
the first liquid inlet is configured to receive a first liquid phase from a source containing the first liquid phase, and
the last liquid inlet is configured to receive a second liquid phase that is immiscible with the first liquid phase from a source containing the second liquid phase.

38. The liquid-liquid chromatographic separator system of claim 37, wherein the first liquid inlet of the first separator stage is fluidically connected to a liquid outlet of at least one of the one or more intermediate separator stages and the source containing the first liquid phase.

39. The liquid-liquid chromatographic separator system of claim 37, wherein the last liquid inlet of the last separator stage is fluidically connected to a liquid outlet of at least one of the one or more intermediate separator stages and the source containing the second liquid phase.

40. The liquid-liquid chromatographic separator system of claim 37, further comprising a mixing region fluidically connected to the first liquid inlet, wherein the mixing region is configured to receive and induce mixing between the first liquid phase and the second liquid phase, thereby forming a mixed liquid stream.

41. The liquid-liquid chromatographic separator system of claim 37, wherein the liquid comprising at least a portion of the first solute and at least a portion of the second solute received by the first liquid inlet is a mixed liquid stream comprising the first liquid phase and the second liquid phase.

42. The liquid-liquid chromatographic separator system of claim 37, further comprising a mixing region fluidically connected to the last liquid inlet, wherein the mixing region is configured to receive and induce mixing between the first liquid phase and the second liquid phase, thereby forming a mixed liquid stream.

43. The liquid-liquid chromatographic separator system of claim 37, wherein the liquid comprising at least a portion of the first solute and at least a portion of the second solute received by the last liquid inlet is a mixed liquid stream comprising the first liquid phase and the second liquid phase.

44. The liquid-liquid chromatographic separator system of claim 37, further comprising a temperature control system configured to control the temperature of the first liquid and phase the second liquid phase.

45. The liquid-liquid chromatographic separator system of claim 35, wherein the feed liquid stream feeds one of the one or more intermediate separator stages before passing through the first separator stage or the last separator stage.

46. The liquid-liquid chromatographic separator system of claim 35, wherein at least one of the one or more intermediate separator stages comprises an intermediate liquid inlet configured to receive a liquid comprising at least a portion of the first solute and at least a portion of the second solute, an intermediate liquid outlet configured to output a liquid having a mole fraction of the first solute relative to the sum of the first solute and the second solute that is larger than a mole fraction of the first solute relative to the sum of the first solute and the second solute in the liquid received by the intermediate liquid inlet, and an intermediate liquid outlet configured to output a liquid having a mole fraction of the second solute relative to the sum of the first solute and the second solute that is larger than a mole fraction of the second solute relative to the sum of the first solute and the second solute in the liquid received by the intermediate liquid inlet.

47. The liquid-liquid chromatographic separator system of claim 35, wherein at least one of the intermediate separator stages comprises an intermediate liquid inlet that is fluidically connected to a liquid outlet of the first separator stage and fluidically connected to a liquid outlet of the last separator stage.

48. The liquid-liquid chromatographic separator system of claim 35, further comprising a mixing region fluidically connected to the intermediate liquid inlet of at least one of the intermediate separator stages, wherein the mixing region is configured to receive and induce mixing between a first liquid phase and a second liquid phase immiscible with the first liquid, thereby forming a mixed liquid stream.

49. The liquid-liquid chromatographic separator system of claim 46, wherein the liquid comprising at least a portion of the first solute and at least a portion of the second solute received by the intermediate liquid inlet is a mixed liquid stream comprising a first liquid phase and a second liquid phase immiscible with the first liquid.

50. The liquid-liquid chromatographic separator system of claim 35, wherein at least one of the three or more separator stages comprises a membrane based separator.

51. The liquid-liquid chromatographic separator system of claim 35, wherein the liquid-liquid chromatographic separator system is configured to be operated continuously.

52. The liquid-liquid chromatographic separator system of claim 37, wherein the first solute has a partition coefficient Ky between the first liquid phase and the second liquid phase of greater than 1.

53. The liquid-liquid chromatographic separator system of claim 37, wherein the second solute has a partition coefficient K₂ between the first liquid phase and the second liquid phase of less than 1.

54. A method, comprising:
transporting a feed liquid stream comprising a first solute and a second solute into a feed liquid inlet of a liquid-liquid chromatographic separator system, wherein the liquid-liquid chromatographic separator system comprises three or more separator stages arranged in series with one another from a first separator stage to a last separator stage, with one or more intermediate stages positioned between the first separator stage and the last separator stage;
transporting a liquid comprising at least a portion of the first solute and at least a portion of the second solute into a first liquid inlet of a first separator stage, such that the first separator stage produces:
a liquid having a mole fraction of the first solute relative to the sum of the first solute and the second solute that is larger than a mole fraction of the first solute relative to the sum of the first solute and the second solute in the liquid received by the first liquid inlet of the first separator stage, and
a liquid having a mole fraction of the second solute relative to the sum of the first solute and the second solute that is larger than a mole fraction of the second solute relative to the sum of the first solute and the second solute in the feed liquid stream; and
transporting a liquid comprising at least a portion of the first solute and at least a portion of the second solute into a last liquid inlet of the last separator stage, such that the last separator stage produces:
a liquid having a mole fraction of the first solute relative to the sum of the first solute and the second solute that is larger than a mole fraction of the first solute relative to the sum of the first solute and the second solute in the feed liquid stream, and
a liquid having a mole fraction of the second solute relative to sum of the first solute and the second solute that is larger than a mole fraction of the second solute relative to the sum of the first solute and the second solute in the liquid received by the last liquid inlet.

55-71. (canceled)
